(19) United States
(12) Patent Application Publication
(10) Pub. No.: US 2009/0062280 A1
(43) Pub. Date: Mar. 5, 2009

(54) Ophthalmic Compositions for Treating Ocular Hypertension

(76) Inventors: James B. Doherty, Montvale, NJ (US); Min Shu, Green Brook, NJ (US); Dong-Ming Shen, Edison, NJ (US); Fengqi Zhang, Edison, NJ (US)

Correspondence Address: MERCK AND CO., INC, P O BOX 2000, RAHWAY, NJ, 07065-0907, US

(21) Appl. No.: 12/225,026

(22) PCT Filed: Mar. 9, 2007
(86) PCT No.: PCT/US2007/006109

§ 371 Date: Sep. 10, 2008

Related U.S. Application Data
(60) Provisional application No. 60/781,904, filed on Mar. 13, 2006.

(51) Int. Cl. A61K 31/5377; C07D 215/227; C07D 413/12; A61K 31/498; C07D 471/04; A61K 31/4375; C07D 241/44; A61K 31/4704

(52) U.S. Cl. 514/235.2; 546/157; 514/312; 544/128; 544/354; 514/249; 514/300; 546/123

(57) ABSTRACT

This invention relates to the use of potent potassium channel blockers or a formulation thereof in the treatment of glaucoma and other conditions which leads to elevated intraocular pressure in the eye of a patient. This invention also relates to the use of such compounds to provide a neuroprotective effect to the eye of mammalian species, particularly humans.

This invention claims the benefit of U.S. Provisional application 60/781,904 filed on Mar. 13, 2006.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

There are several current therapies for treating glaucoma and elevated intraocular pressure (e.g., pilocarpine, beta blockers (e.g., timolol), carbonic anhydrase inhibitors (e.g., dorzolamide, brinzolamide) and prostaglandins (e.g., latanoprost), but the efficacy and the side effect profiles of these agents are not ideal. Recently potassium channel blockers were found to reduce intraocular pressure in the eye and therefore provide yet one more approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Opthalmol. Vis. Sci 38, 1997; WO 89/10757, WO94/28900, and WO 96/33719).

### SUMMARY OF THE INVENTION

This invention relates to the use of potent quinoli-2(1H)-one derivatives and their aza analogues as potassium channel blockers or a formulation thereof in the treatment of glaucoma and other conditions which are related to elevated intraocular pressure in the eye of a patient. This invention also relates to the use of such compounds to provide a neuroprotective effect to the eye of mammalian species, particularly humans. More particularly this invention relates to the treatment of glaucoma and/or ocular hypertension (elevated intraocular pressure) using novel quinoli-2(1H)-one derivatives and their aza analogues having the structural formula I:

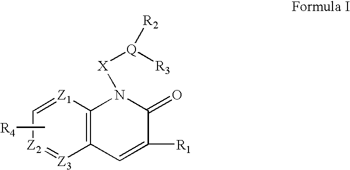

or a pharmaceutically acceptable salt, ester including phosphate, enantiomer, diastereomer or mixture thereof:
wherein,
Z, Z₁, Z₂, and Z₃ independently represent CH or N;
R and R^{y} independently represent hydrogen, or C₁₋₆ alkyl;
R₁ represents hydrogen or C₁₋₆ alkyl, CF₃, (CH₂)ₙC₃₋₁₀ cycloalkyl, (CH₂)ₙC₆₋₁₀ aryl, —(CH₂)ₙC₅₋₁₀ heteroaryl, C₁₋₆ alkoxy, OH, COR^{C}, said alkyl, cycloalkyl, aryl, heteroaryl, and alkoxy optionally substituted with 1-3 groups selected from R^{b};
X represents —(CHR₇)ₚ—, —(CR₇)ₚC(O)—;
Q represents N, CR^{y}, or O, wherein R₂ is absent when Q is O;
R₂ represents hydrogen, C₁₋₁₀ alkyl, C₂₋₁₀ hydroxylalkyl, C₁₋₆ alkylSR, —(CH₂)ₙO(CH₂)ₘOR, (CH₂)ₘOR, —(CH₂)ₙ(CHR₇)ₛ(CH₂)ₘC₁₋₆ alkoxy, —(CH₂)ₙ(CHR₇)(CH₂)ₘC₃₋₈ cycloalkyl, —(CH₂)ₙ(CHR₇)ₛ(CH₂)ₘC₃₋₁₀ heterocyclyl, —(CH₂)ₙC₅₋₁₀ heteroaryl, —N(R)₂, —COOR, or —(CH₂)ₙ(CHR₇)ₛ(CH₂)ₘC₆₋₁₀ aryl, said allyl, cycloalkyl, heterocyclyl, aryl or heteroaryl optionally substituted with 1-3 groups selected from R^{a};
R₃ represents hydrogen, C₁₋₁₀ alkyl, C₂₋₆ alkenyl, —(CH₂)ₙ(CHR₇)ₛ(CH₂)ₘC₃₋₈ cycloalkyl, —(CH₂)ₙ(CHR₇)ₛ(CH₂)ₘC₃₋₁₀ heterocyclyl, —(CH₂)ₙ(CHR₇)(CH₂)ₘC₅₋₁₀ heteroaryl, —(CH₂)ₙ(CHR₇)ₛ(CH₂)ₘCOOR, —(CH₂)ₙ(CHR₇)ₛ(CH₂)ₘC₆₋₁₀ aryl, —(CH₂)ₙ(CHR₇)ₛ(CH₂)ₘNHR₈, —(CH₂)ₙ(CHR₇)ₛ(CH₂)ₘN(R)₂, —(CH₂)ₙ(CHR₇)ₛ(CH₂)ₘN(R₈)₂, —(CH₂)ₙ(CHR₇)ₛ(CH₂)ₘNHCOOR, —(CH₂)ₙ(CHR₇)ₛ(CH₂)ₘN(R₈)CO₂R, —(CH₂)ₙ(CHR₇)ₛ(CH₂)ₘN(R₈)COR, —(CH₂)ₙ(CHR₇)ₛ(CH₂)ₘNHCOR, —(CH₂)ₙ(CHR₇)ₛ(CH₂)ₘCONH(R₈), aryl, —(CH₂)ₙ(CHR₇)ₛ(CH₂)ₘOR, —(CH₂)ₙC(R₇)₂(CH₂)ₘOR, CF₃, —(CH₂)ₙ(CHR₇)ₛ(CH₂)ₘSO₂R, —(CH₂)ₙ(CHR₇)ₛ(CH₂)ₘSO₂N(R)₂, —(CH₂)ₙ(CHR₇)ₛ(CH₂)ₘCON(R)₂, —(CH₂)ₙ(CHR₇)ₛ(CH₂)ₘCONHC(R)₃, —(CH₂)ₙCONHC(R)₂CO₂R, —(CH₂)ₙ(CHR₇)ₛ(CH₂)ₘCOR₈, nitro, cyano or halogen, said alkyl, cycloalkyl, alkoxy, heterocyclyl, aryl or heteroaryl optionally substituted with 1-3 groups of R^{a};
or, when Q is N, R₂ and R₃ taken together with the intervening N atom form a 4-10 membered heterocyclic ring optionally interrupted by 1-2 atoms of O, S, C(O) or NR, and optionally having 1-4 double bonds, and optionally substituted by 1-3 groups selected from R^{a};
or, when Q equals CR^{y}, R₂ and R₃ taken together with the intervening CR^{y} form a 4-10 membered carbocyclic or heterocyclic aromatic ring or fused ring optionally interrupted by 1-2 atoms of O, S, C(O) or NR, and optionally having 1-5 double bonds, and optionally substituted by 1-3 groups selected from R^{a};
R₄ represents hydrogen, C₁₋₆ alkoxy, halogen, cyano, OH, C₁₋₆ alkyl, COOR, SO₃H, C₁₋₆ alkylcarbonyl, S(O)_{q}R^{y}, —O(CH₂)ₙN(R)₂, —O(CH₂)ₙCO₂R, —OPO(OH)₂, CF₃, —N(R)₂, nitro, or C₁₋₆ alkylamino;
R₇ represents hydrogen, C₁₋₆ alkyl, —(CH₂)ₙCOOR or —(CH₂)ₙN(R)₂,
R₈ represents —(CH₂)ₙC₃₋₈ cycloalkyl, —(CH₂)_{n 3-10} heterocyclyl, C₁₋₆ alkoxy or —(CH₂)ₙC₅₋₁₀ heteroaryl, —(CH₂)ₙC₆₋₁₀ aryl said heterocyclyl, cycloalkyl, aryl or heteroaryl optionally substituted with 1-3 groups selected from R^{a};
R^{a} represents F, Cl, Br, I, CF₃, N(R)₂, NO₂, CN, —(CH₂)ₙCOR₈, —(CH₂)ₙCONHR₈, —(CH₂)ₙCON(R₈)₂, —O(CH₂)ₙCOOR, —NH(CH₂)ₙOR, —COOR, —OCF₃, —O—, —NHCOR, —SO₂R, —SO₂NR₂, —SR, (C₁-C₆ alkyl)O—, —(CH₂)ₙ—O—(CH₂)ₘOR, —(CH₂)ₙC₁₋₆ alkoxy, (aryl)O—, —OH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —(C₁-C₆ alkyl)-C₃₋₁₀ heterocyclyl-R_{w}, —(CH₂)ₙ—K—C(═K)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-K—C(═K)N(R)₂, —(CH₂)ₙSO₂R, —(CH₂)ₙSO₃H, —(CH₂)ₙPO(OR)₂, —(CH₂)ₙOPO(OR)₂, cyclohexyl, cyclopentyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, and COOR;
K independently represents CH, CH₂ or NH;
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R^{b} represents C₁₋₆ alkyl, —COOR, —SO₃R, CN, (CH₂)ₙOR, C(O)O(CH₂)ₙC(O)R, —OPO(OH)₂, —(CH₂)ₙC₆₋₁₀ aryl, or —(CH₂)ₙC₅₋₁₀ heteroaryl;
R^{c} represents hydrogen, C₁₋₆ alkyl, or —(CH₂)ₙC₆₋₁₀ aryl;
m is 0-3;
n is 0-3;
q is 0-2;
s is 0-1; and
p is 0-2.

This and other aspects of the invention will be realized upon inspection of the invention as a whole.

### DETAILED DESCRIPTION OF THE INVENTION

The present invention is directed to novel potassium channel blockers of Formula I. It also relates to a method for decreasing elevated intraocular pressure or treating glaucoma by administration, preferably topical or intra-camaral administration, of a composition containing a potassium channel blocker of Formula I described hereinabove and a pharmaceutically acceptable carrier.

In an embodiment of the instant invention are the compounds wherein

X represents CHR₇CO.

Still another embodiment of this invention is realized when Q is N and all other variables are as originally described.

Still another embodiment of this invention is realized when Q is CH or CCH₃ and all other variables are as originally described.

In another embodiment R_{w} is selected from H, C₁₋₆ alkyl, —C(O)C₁₋₆ alkyl and —C(O)N(R)₂.

Another embodiment of this invention is realized when Z=N, and Z₁, Z₂, and Z₃ are each CH and all other variables are as originally described. A sub-embodiment of this invention is realized when QR₂R₃ is a dialkylamine or hydroxylamine and all other variables are as originally described.

Still another embodiment of this invention is realized when R₁ is C₁₋₆ alkyl, Z is N, Z₁, Z₂, and Z₃ are each CH, and QR₂R₃ is a dialkylamine or hydroxyldialkylamine and all other variables are as originally described.

Yet another embodiment of this invention is realized when R₇ is hydrogen or C₁₋₆ alkyl, and all other variables are as originally described.

Another embodiment of the instant invention is realized when R^{a} is selected from F, Cl, Br, I, CF₃, N(R)₂, NO₂, CN, —CONHR₈, —CON(R₈)₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙ—O—(CH₂)ₘOR, —(CH₂)ₙC₁₋₆ alkoxy, (aryl)O—, —OH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(CH₂)ₙ—K—C(═K)N(R)₂, —(C₂₋₆ alkenyl)NR_{w}(CH₂)ₙC₃₋₁₀ heterocyclyl-R_{w}, —(C₂₋₆ alkenyl)-K—C(═K)N(R)₂, —(CH₂)ₙSO₂R, —(CH₂)ₙSO₃H, —(CH₂)ₙPO(OR)₂, C₂₋₆ alkenyl, and C₁-C₁₀ alkyl, said alkyl and alkenyl, optionally substituted with 1-3 groups selected from C₁-C₆ alkyl, and COOR;

Still another embodiment of this invention is realized when R₂ and R₃ are taken together with the intervening N atom form a 4-10 membered heterocyclic carbon ring optionally interrupted by 1-2 atoms of O, S, C(O) or NR, and optionally having 1-4 double bonds, and optionally substituted by 1-3 groups selected from R^{a}. Examples of said heterocyclic groups are:

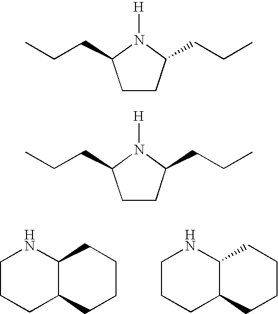

and the like.

Still another embodiment of this invention is realized when Q equals CR^{y}, and R₂ and R₃ taken together with the intervening CR^{y} form a 4-10 membered carbocyclic or heterocyclic aromatic ring or fused ring optionally interrupted by 1-2 atoms of O, S, C(O) or NR, and optionally having 1-5 double bonds, and optionally substituted by 1-3 groups selected from R^{a}. Examples of said groups are phenyl, pyridinyl, adamantyl, [1.1.1]bicyclopentyl, and the like.

Another embodiment of this invention is realized by structural formula II:

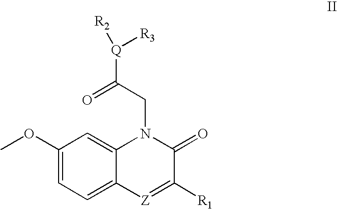

or a pharmaceutically acceptable salt, enantiomer, diastereomer or mixture thereof:
wherein,

### Q is N;

### Z=CH or N;

R₁ represents hydrogen or C₁₋₆ alkyl, (CH₂)ₙC₃₋₁₀ cycloalkyl, (CH₂)ₙC₆₋₁₀ aryl, —(CH₂)ₙC₅₋₁₀ heteroaryl, C₁₋₆ alkoxy, said alkyl, cycloalkyl, aryl and alkoxy optionally substituted with 1-3 groups selected from R^{b};
R₂ represents hydrogen, C₁₋₁₀ alkyl, C₂₋₁₀ hydroxylalkyl, (CH₂)ₘOR, —(CH₂)ₙ(CHR₇)ₛ(CH₂)ₘC₁₋₆ alkoxy, —(CH₂)ₙ(CHR₇)S(CH₂)ₘC₃₋₈ cycloalkyl, —(CH₂)ₙ(CHR₇)ₛ(CH₂)ₘC₃₋₁₀ heterocyclyl, —(CH₂)ₙC₅₋₁₀ heteroaryl, or —(CH₂)ₙ(CHR₇)ₛ(CH₂)ₘC₆₋₁₀ aryl, said alkyl, cycloalkyl, heterocyclyl, aryl or heteroaryl optionally substituted with 1-3 groups selected from R^{a};
R₃ represents hydrogen, C₁₋₁₀ alkyl, —(CH₂)ₙ(CHR₇)ₛ(CH₂)ₘC₃₋₈ cycloalkyl, —(CH₂)ₙ(CHR₇)ₛ(CH₂)ₘC₃₋₁₀ heterocyclyl, —(CH₂)ₙ(CHR₇)ₛ(CH₂)ₘC₅₋₁₀ heteroaryl, or —(CH₂)ₙ(CHR₇)ₛ(CH₂)ₘC₆₋₁₀ aryl, said alkyl, cycloalkyl, alkoxy, heterocyclyl, aryl or heteroaryl optionally substituted with 1-3 groups of R^{a}; and all other variables are as described herein.

A sub-embodiment of the compounds of formula II is realized when R₁ is C₁₋₆ alkyl, optionally substituted with 1 to 3 groups of R^{b}. Examples of C₁₋₆ alkyls are t-butyl, ethyl, isopropyl, methyl and the like. Another sub-embodiment of the compounds of formula II is realized when R₁ is hydrogen. Still another sub-embodiment of the compounds of formula II is realized when R₁ is (CH₂)ₙC₆₋₁₀ aryl, optionally substituted with 1 to 3 groups of R_{b}. Yet another sub-embodiment of the compounds of formula II is realized when R₁ is (CH₂)ₙC₃₋₁₀ cycloalkyl, optionally substituted with 1 to 3 groups of R_{b}.

Another sub-embodiment of the compounds of formula II is realized when R₂ and R₃ are independently C₁₋₁₀ alkyl, —(CH₂)ₙ(CHR₇)ₛ(CH₂)ₘC₆₋₁₀ aryl, (CH₂)ₙ(CHR₇)ₛ(CH₂)ₘC₃₋₁₀ heterocyclyl, said alkyl, heterocyclyl, aryl optionally substituted with 1-3 groups selected from R^{a}.

Another sub-embodiment of the compounds of formula II is realized when R₂ and R₃ are independently hydrogen, C₁₋₁₀ alkyl, said alkyl, optionally substituted with 1-3 groups selected from R^{a}.

Examples of compounds to be used in this invention are
N,N-Dibutyl-2-(3-tert-butyl-7-methoxy-2-oxoquinolin-1(2H)-yl)acetamide,
2-(3-Tert-butyl-7-methoxy-2-oxoquinolin-1(2H)-yl)-N,N-bis(3-methylbutyl)acetamide,
2-(3-Tert-butyl-7-methoxy-2-oxoquinolin-1(2H)-yl)-N-(cyclopropylmethyl)-N-propylacetamide,
2-(3-Tert-butyl-7-methoxy-2-oxoquinolin-1(2H)-yl)-N,N-diisobutylacetamide,
2-(3-Tert-butyl-7-methoxy-2-oxoquinolin-1(2H)-yl)-N-(3,3-dimethylbutyl)-N-ethylacetamide,
2-(3-Tert-butyl-7-methoxy-2-oxoquinolin-1(2H)-yl)-N-(3,3-dimethylbutyl)-N-propylacetamide,
2-(3-Tert-butyl-7-methoxy-2-oxoquinolin-1(2H)-yl)-N-(2,2-dimethylpropyl)-N-ethylacetamide,
2-(3-Tert-butyl-7-methoxy-2-oxoquinolin-1(2H)-yl)-N-ethyl-NV-(3-methylbutyl)acetamide,
2-(3-Tert-butyl-7-methoxy-2-oxoquinolin-1(2H)-yl)-N-(3-methylbutyl)-N-propylacetamide,
N-Butyl-2-(3-tert-butyl-7-methoxy-2-oxoquinolin-1(2H)-yl)-N-(3-methylbutyl)acetamide,
2-(3-Ter-butyl-7-methoxy-2-oxoquinolin-1(2H)-yl)-N-(2-hydroxyethyl)-N-(3-methylbutyl)acetamide,
2-(3-Tert-butyl-7-methoxy-2-oxoquinolin-1(2H)-yl)-N-isobutyl-N-propylacetamide,
N-butyl-2-(3-tert-butyl-7-methoxy-2-oxoquinolin-1(2H)-yl)-N-isobutylacetamide,
2-(3-Tert-butyl-7-methoxy-2-oxoquinolin-1(2H)-yl)-N-(3-hydroxy-2,2-dimethylpropyl)-N-(3-methylbutyl)acetamide,
2-(3-Tert-butyl-7-methoxy-2-oxoquinolin-1(2H)-yl)-N-(5-hydroxy-4,4-dimethylpentyl)-N-(3-methylbutyl)acetamide,
2-(3-Tert-butyl-7-methoxy-2-oxoquinolin-1(2H)-yl)-N-(3,3-dimethylbutyl)-N-(3-hydroxy-2,2-dimethylpropyl)acetamide,
2-(3-Tert-butyl-7-methoxy-2-oxoquinolin-1(2H)-yl)-N-(3,3-dimethylbutyl)-N-(2-hydroxy-2-methylpropyl)acetamide,
2-(3-Tert-butyl-7-methoxy-2-oxoquinolin-1(2H)-yl)-N-(5-hydroxypentyl)-N-(3-methylbutyl)acetamide,
2-(3-Tert-butyl-7-methoxy-2-oxoquinolin-1(2H)-yl)-N-(3,3-dimethylbutyl)-N-(5-hydroxypentyl)acetamide,
2-(3-Tert-butyl-7-methoxy-2-oxoquinolin-1(2H)-yl)-N-(3,3-dimethylbutyl)-N-(2-hydroxy-1,1-dimethylethyl)acetamide,
3-Tert-butyl-1-(3,3-dimethyl-2-oxobutyl)-7-methoxyquinolin-2(1H)-one,
N,N-Dibutyl-2-(3-isopropyl-7-methoxy-2-oxoquinolin-1(2H)-yl)acetamide,
N,N-Diisobutyl-2-(3-isopropyl-7-methoxy-2-oxoquinolin-1(2H)-yl)acetamide,
N-(3,3-Dimethylbutyl)-N-ethyl-2-(3-isopropyl-7-methoxy-2-oxoquinolin-1(2H)-yl)acetamide,
N-(3,3-Dimethylbutyl)-2-(3-isopropyl-7-methoxy-2-oxoquinolin-1(2H)-yl)-N-propylacetamide,
2-(3-Isopropyl-7-methoxy-2-oxoquinolin-1(2H)-yl)-N-octyl-N-pentylacetamide,
N-Ethyl-2-(3-isopropyl-7-methoxy-2-oxoquinolin-1(2H)-yl)-N-1,3-thiazol-2-ylacetamide,
(±)-3-Isopropyl-7-methoxy-1-{2-[trans-octahydroisoquinolin-2(1H)-yl]-2-oxoethyl} quinolin-2(1H)-one,
N,N-Bis(2,2-dimethylpropyl)-2-(3-isopropyl-7-methoxy-2-oxoquinolin-1(2H)-yl)acetamide,
2-(3-Isopropyl-7-methoxy-2-oxoquinolin-1(2H)-yl)-N-(3-methylbutyl)-N-propylacetamide,
N-Butyl-2-(3-isopropyl-7-methoxy-2-oxoquinolin-1(2H)-yl)-N-(3-methylbutyl)acetamide,
N-Isobutyl-2-(3-isopropyl-7-methoxy-2-oxoquinolin-1(2H)-yl)-N-propylacetamide,
N-Butyl-N-isobutyl-2-(3-isopropyl-7-methoxy-2-oxoquinolin-1(2H)-yl)acetamide,
N-(5-Hydroxy-4,4-dimethylpentyl)-2-(3-isopropyl-7-methoxy-2-oxoquinolin-1(2H)-yl)-N-(3-methylbutyl)acetamide,
N-(3,3-Dimethylbutyl)-N-(5-hydroxypentyl)-2-(3-isopropyl-7-methoxy-2-oxoquinolin-1(2H)-yl)acetamide,
N,N-Dibutyl-2-(3-cyclohexyl-7-methoxy-2-oxoquinolin-1(2H)-yl)acetamide,
2-(3-Cyclohexyl-7-methoxy-2-oxoquinolin-1(2H)-yl)-N,N-dipropylacetamide,
2-(3-Cyclohexyl-7-methoxy-2-oxoquinolin-1(2H)-yl)-N-(cyclopropylmethyl)-N-propylacetamide,
N-Cyclohexyl-2-(3-cyclohexyl-7-methoxy-2-oxoquinolin-1(2H)-yl)-N-ethylacetamide,
2-(3-Cyclohexyl-7-methoxy-2-oxoquinolin-1(2H)-yl)-N,N-diisobutylacetamide,
2-(3-Cyclohexyl-7-methoxy-2-oxoquinolin-1(2H)-yl)-N,N-bis(3-methylbutyl)acetamide,
2-(3-Cyclohexyl-7-methoxy-2-oxoquinolin-1(2H)-yl)-N-ethyl-N-(3-methylbutyl)acetamide,
2-(3-Cyclohexyl-7-methoxy-2-oxoquinolin-1(2H)-yl)-N-(3,3-dimethylbutyl)-N-ethylacetamide,
2-(3-Cyclohexyl-7-methoxy-2-oxoquinolin-1(2H)-yl)-N-(3,3-dimethylbutyl)-N-propylacetamide,
2-(3-Cyclohexyl-7-methoxy-2-oxoquinolin-1(2H)-yl)-N-(2,2-dimethylpropyl)-N-ethylacetamide,
2-(3-Cyclohexyl-7-methoxy-2-oxoquinolin-1(2H)-yl)-N-(3-methylbutyl)-N-propylacetamide,
N-Butyl-2-(3-cyclohexyl-7-methoxy-2-oxoquinolin-1(2H)-yl)-N-(3-methylbutyl)acetamide,
2-(3-Cyclohexyl-7-methoxy-2-oxoquinolin-1(2H)-yl)-N-methyl-N-(3-methylbutyl)acetamide,
2-(3-Cyclohexyl-7-methoxy-2-oxoquinolin-1(2H)-yl)-N-(2-hydroxyethyl)-N-(3-methylbutyl)acetamide,
2-(3-Cyclohexyl-7-methoxy-2-oxoquinolin-1(2H)-yl)-N-ethyl-N-isobutylacetamide,
2-(3-Cyclohexyl-7-methoxy-2-oxoquinolin-1(2H)-yl)-N-isobutyl-N-propylacetamide,
N-Butyl-2-(3-cyclohexyl-7-methoxy-2-oxoquinolin-1(2H)-yl)-N-isobutylacetamide,
2-(3-Cyclohexyl-7-methoxy-2-oxoquinolin-1(2H)-y)-N-(3-hydroxy-2,2-dimethylpropyl)-N-(3-methylbutyl)acetamide,
N-(3,3-Dimethylbutyl)-2-(7-methoxy-2-oxo-3-phenylquinolin-1(2H)-yl)-N-propylacetamide
N,N-Dibutyl-2-(7-methoxy-2-oxo-3-phenylquinolin-1(2H)-yl)acetamide,
N-(Cyclopropylmethyl)-2-(7-methoxy-2-oxo-3-phenylquinolin-1(2H)-yl)-N-propylacetamide,
N,N-Diisobutyl-2-(7-methoxy-2-oxo-3-phenylquinolin-1(2A)-yl)acetamide,
2-(7-Methoxy-2-oxo-3-phenylquinolin-1(2H)-yl)-N,N-bis(3-methylbutyl)acetamide,
N-Ethyl-2-(7-methoxy-2-oxo-3-phenylquinolin-1(2H)-yl)-N-(3-methylbutyl)acetamide,
N-(3,3-Dimethylbutyl)-N-ethyl-2-(7-methoxy-2-oxo-3-phenylquinolin-1(2H)-yl)acetamide,
N-(2,2-Dimethylpropyl)-N-ethyl-2-(7-methoxy-2-oxo-3-phenylquinolin-1(2H)-yl)acetamide,
2-(3-Ethyl-7-methoxy-2-oxoquinolin-1(2H)-yl)-N,N-bis(3-methylbutyl)acetamide,
N-(3,3-Dimethylbutyl)-2-(3-ethyl-7-methoxy-2-oxoquinolin-1(2H)-yl)-N-propylacetamide,
N,N-Bis(3,3-dimethylbutyl)-2-(3-ethyl-7-methoxy-2-oxoquinolin-1(2H)-yl)acetamide,
N,N-Bis(2,2-dimethylpropyl)-2-(3-ethyl-7-methoxy-2-oxoquinolin-1(2H)-yl)acetamide,
N-Butyl-N-(2,2-dimethylpropyl)-2-(3-ethyl-7-methoxy-2-oxoquinolin-1(2H)-yl)acetamide,
N-(2,2-Dimethylpropyl)-2-(3-ethyl-7-methoxy-2-oxoquinolin-1(2H)-yl)-N-pentylacetamide,
N-(2,2-Dimethylpropyl)-2-(3-ethyl-7-methoxy-2-oxoquinolin-1(2H)-yl)-N-(3-methylbutyl)acetamide,
N-Cyclopentyl-N-(2,2-dimethylpropyl)-2-(3-ethyl-7-methoxy-2-oxoquinolin-1(2H)-yl)acetamide,
N-(3,3-Dimethylbutyl)-N-(2,2-dimethylpropyl)-2-(3-ethyl-7-methoxy-2-oxoquinolin-1(2H)-yl)acetamide,
N-Butyl-N-(3,3-dimethylbutyl)-2-(3-ethyl-7-methoxy-2-oxoquinolin-1(2H)-yl)acetamide,
N-(3,3-Dimethylbutyl)-2-(3-ethyl-7-methoxy-2-oxoquinolin-1(2H)-yl)-N-pentylacetamide,
N-Cyclopentyl-N-(3,3-dimethylbutyl)-2-(3-ethyl-7-methoxy-2-oxoquinolin-1(2H)-yl)acetamide,
N-Butyl-2-(3-ethyl-7-methoxy-2-oxoquinolin-1(2H)-yl)-N-(3-methylbutyl)acetamide,
N,N-Dibutyl-2-(3-cyclopentyl-7-methoxy-2-oxoquinolin-1(2H)-yl)acetamide,
2-(3-Cyclopentyl-7-methoxy-2-oxoquinolin-1(2H)-yl)-N,N-dipropylacetamide,
2-(3-Cyclopentyl-7-methoxy-2-oxoquinolin-1(2H)-yl)-N-(cyclopropylmethyl)-N-propylacetamide,
N-Cyclohexyl-2-(3-cyclopentyl-7-methoxy-2-oxoquinolin-1(2H)-yl)-N-ethylacetamide,
N-Butyl-2-(3-cyclopentyl-7-methoxy-2-oxoquinolin-1(2H)-yl)-N-ethylacetamide,
2-(3-Cyclopentyl-7-methoxy-2-oxoquinolin-1(2H)-yl)-N,N-diisobutylacetamide,
2-(3-Cyclopentyl-7-methoxy-2-oxoquinolin-1(2B-yl)-N,N-bis(3-methylbutyl)acetamide,
2-(3-Cyclopentyl-7-methoxy-2-oxoquinolin-1(2H)-yl)-N-ethyl-N-(3-methylbutyl)acetamide,
N-Butyl-2-(3-cyclopentyl-7-methoxy-2-oxoquinolin-1(2R)-yl)-N-propylacetamide,
2-(3-Cyclopentyl-7-methoxy-2-oxoquinolin-1(2H)-yl)-N-(3,3-dimethylbutyl)-N-ethylacetamide,
2-(3-Cyclopentyl-7-methoxy-2-oxoquinolin-1(2H)-yl)-N-(3,3-dimethylbutyl)-N-propylacetamide,
2-(3-Cyclopentyl-7-methoxy-2-oxoquinolin-1(2H)-yl)-N-(2,2-dimethylpropyl)-N-ethylacetamide,
N-Cyclohexyl-2-(3-cyclopentyl-7-methoxy-2-oxoquinolin-1(2H)-yl)-N-isopropylacetamide,
2-(3-Cyclopentyl-7-methoxy-2-oxoquinolin-1(2H)-yl)-N,N-bis(3,3-dimethylbutyl)acetamide,
2-(3-Cyclopentyl-7-methoxy-2-oxoquinolin-1(2R)-yl)-N-octyl-N-pentylacetamide,
N-(sec-Butyl)-2-(3-cyclopentyl-7-methoxy-2-oxoquinolin-1(2H)-yl)-N-propylacetamide,
2-(3-Cyclopentyl-7-methoxy-2-oxoquinolin-1(2H)-yl)-N-ethyl-N-1,3-thiazol-2-ylacetamide,
(±)-3-Cyclopentyl-7-methoxy-1-{2-trans-octahydroisoquinolin-2(1H)-yl)-2-oxoethyl} quinolin-2(1H)-one,
N-Butyl-2-(3-cyclopentyl-7-methoxy-2-oxoquinolin-1(2H)-yl)-N-(2,2-dimethylpropyl)acetamide,
2-(3-Cyclopentyl-7-methoxy-2-oxoquinolin-1(2H)-yl)-N-(2,2-dimethylpropyl)-N-pentylacetamide,
2-(3-Cyclopentyl-7-methoxy-2-oxoquinolin-1(2H)-yl)-N-(2,2-dimethylpropyl)-N-(3-methylbutyl)acetamide,
N-Cyclopentyl-2-(3-cyclopentyl-7-methoxy-2-oxoquinolin-1(2H)-yl)-N-(2,2-dimethylpropyl)acetamide,
2-(3-Cyclopentyl-7-methoxy-2-oxoquinolin-1(2H)-yl)-N-(3,3-dimethylbutyl)-N-(2,2-dimethylpropyl)acetamide,
N-Butyl-2-(3-cyclopentyl-7-methoxy-2-oxoquinolin-1(2H)-yl)-N-(3,3-dimethylbutyl)acetamide,
2-(3-Cyclopentyl-7-methoxy-2-oxoquinolin-1(2H)-yl)-N-(3,3-dimethylbutyl)-N-pentylacetamide,
N-Cyclopentyl-2-(3-cyclopentyl-7-methoxy-2-oxoquinolin-1(2H)-yl)-N-(3,3-dimethylbutyl)acetamide,
2-(3-Cyclopentyl-7-methoxy-2-oxoquinolin-1(2H)-yl)-N-(3,3-dimethylbutyl)-N-(3-methylbutyl)acetamide,
2-(3-Cyclopentyl-7-methoxy-2-oxoquinolin-1(2H)-yl)-N-(3-methylbutyl)-N-propylacetamide,
N-Butyl-2-(3-cyclopentyl-7-methoxy-2-oxoquinolin-1(2H)-yl)-N-(3-methylbutyl)acetamide,
2-(3-Cyclopentyl-7-methoxy-2-oxoquinolin-1(2H)-yl)-N-methyl-N-(3-methylbutyl)acetamide,
2-(3-Cyclopentyl-7-methoxy-2-oxoquinolin-1(2H)-yl)-N-(2-hydroxyethyl)-N-(3-methylbutyl)acetamide,
2-(3-Cyclopentyl-7-methoxy-2-oxoquinolin-1(2H)-yl)-N-ethyl-N-isobutylacetamide,
2-(3-Cyclopentyl-7-methoxy-2-oxoquinolin-1(2H)-yl)-N-isobutyl-N-propylacetamide,
N-Butyl-2-(3-cyclopentyl-7-methoxy-2-oxoquinolin-1(2H)-yl)-N-isobutylacetamide,
2-(3-Cyclopentyl-7-methoxy-2-oxoquinolin-1(2H)-yl)-N-(3-hydroxy-2,2-dimethylpropyl)-N-(3-methylbutyl)acetamide,
2-(3-Cyclopentyl-7-methoxy-2-oxoquinolin-1(2H)-yl)-N-(5-hydroxy-4,4-dimethylpentyl)-N-(3-methylbutyl)acetamide,
2-(3-Cyclopentyl-7-methoxy-2-oxoquinolin-1(2H)-yl)-N-(3,3-dimethylbutyl)-N-(5-hydroxypentyl)acetamide,
N,N-Dibutyl-2-(2-tert-butyl-6-methoxy-3-oxopyrido[2,3-b]pyrazin-4(3H)-yl)acetamide,
2-(2-tert-Butyl-6-methoxy-3-oxopyrido[2,3-b]pyrazin-4(3H)-yl)-N-(3,3-dimethylbutyl)-N-propylacetamide,
2-(2-tert-Butyl-6-methoxy-3-oxopyrido[2,3-b]pyrazin-4(3H)-yl)-N-ethyl-N-(3-methylbutyl)acetamide,
2-(2-tert-Butyl-6-methoxy-3-oxopyrido[2,3-b]pyrazin-4(3H)-yl)-N,N-bis(3-methylbutyl)acetamide,
2-(2-tert-Butyl-6-methoxy-3-oxopyrido[2,3-b]pyrazin-4(3H)-yl)-N,N-diisobutylacetamide,
N-Butyl-2-(2-tert-butyl-6-methoxy-3-oxopyrido[2,3-b]pyrazin-4(3H)-yl)-N-propylacetamide,
2-(2-tert-Butyl-6-methoxy-3-oxopyrido[2,3-b]pyrazin-4(3R)-yl)-N-(3,3-dimethylbutyl)-N-ethylacetamide, 2-(2-tert-Butyl-6-methoxy-3-oxopyrido[2,3-b]pyrazin-4(3H)-yl)-N-ethyl-N-1,3-thiazol-2-ylacetamide,
2-(2-tert-Butyl-6-methoxy-3-oxopyrido[2,3-b]pyrazin-4(3H)-yl)-N,N-bis(2,2-dimethylpropyl)acetamide,
2-(3-tert-Butyl-7-methoxy-2-oxoquinoxalin-1(2H)-yl)-N-(3,3-dimethylbutyl)-N-propylacetamide,
N,N-Dibutyl-2-(3-tert-butyl-7-methoxy-2-oxoquinoxalin-1(2H)-yl)acetamide,
2-(3-tert-Butyl-7-methoxy-2-oxoquinoxalin-1(2H)-yl)-N,N-diisobutylacetamide,
N-Butyl-2-(3-tert-butyl-7-methoxy-2-oxoquinoxalin-1(2H)-yl)-N-propylacetamide,
2-(3-tert-Butyl-7-methoxy-2-oxoquinoxalin-1(2H)-yl)-N-(cyclopropylmethyl)-N-propylacetamide,
2-(3-tert-Butyl-7-methoxy-2-oxoquinoxalin-1(2H)-yl)-N,N-dipropylacetamide,
2-(3-tert-Butyl-7-methoxy-2-oxoquinoxalin-1(2H)-yl)-N-(3,3-dimethylbutyl)-N-ethylacetamide,
2-(3-tert-Butyl-7-methoxy-2-oxoquinoxalin-1(2H)-yl)-N-ethyl-N-(3-methylbutyl)acetamide,
2-(3-tert-Butyl-7-methoxy-2-oxoquinoxalin-1(2H)-yl)-N-(2,2-dimethylpropyl)-N-ethylacetamide,
N-Butyl-2-(3-tert-butyl-7-methoxy-2-oxoquinoxalin-1(2H)-yl)-N-ethylacetamide,
2-(3-tert-Butyl-7-methoxy-2-oxoquinoxalin-1(2H)-yl)-N-cyclohexyl-N-ethylacetamide,
2-(3-tert-Butyl-7-methoxy-2-oxoquinoxalin-1(2H)-yl)-N-ethyl-N-1,3-thiazol-2-ylacetamide,
3-tert-Butyl-7-methoxy-1-{2-cis-octahydroisoquinolin-2(1H)-yl]-2-oxoethyl} quinoxalin-2(1H)-one,
3-tert-Butyl-7-methoxy-1-{2-cis-octahydroisoquinolin-2(1H)-yl]-2-oxoethyl} quinoxalin-2(1H)-one,
2-(3-tert-Butyl-7-methoxy-2-oxoquinoxalin-1(2H)-yl)-N,N-bis(2,2-dimethylpropyl)acetamide,
2-(3-tert-Butyl-7-methoxy-2-oxoquinoxalin-1(2H)-yl)-N-(2-hydroxyethyl)-N-(3-methylbutyl)acetamide,
2-(3-tert-Butyl-7-methoxy-2-oxoquinoxalin-1(2H)-yl)-N-(3,3-dimethylbutyl)-N-(3-hydroxy-2,2-dimethylpropyl)acetamide,
2-(3-tert-Butyl-7-methoxy-2-oxoquinoxalin-1(2H)-yl)-N-(3-hydroxy-2,2-dimethylpropyl)-N-(3-methylbutyl)acetamide,
2-(3-tert-Butyl-7-methoxy-2-oxoquinoxalin-1(2H)-yl)-N-(3,3-dimethylbutyl)-N-(4-hydroxy-3,3-dimethylbutyl)acetamide,
2-(3-tert-Butyl-7-methoxy-2-oxoquinoxalin-1(2H)-yl)-N-(3,3-dimethylbutyl)-N-(2-hydroxyethyl)acetamide,
2-(3-tert-Butyl-7-methoxy-2-oxoquinoxalin-1(2H)-yl)-N-(3,3-dimethylbutyl)-N-(3-hydroxy-3-methylbutyl)acetamide,
2-(3-tert-Butyl-7-methoxy-2-oxoquinoxalin-1(2H)-yl)-N-(3,3-dimethylbutyl)-N-(3-methylbut-2-en-1-yl)acetamide,
2-(3-tert-Butyl-7-methoxy-2-oxoquinoxalin-1(2H)-yl)-N-(3,3-dimethylbutyl)-N-(2-hydroxy-2-methylpropyl)acetamide,
2-(3-tert-Butyl-7-methoxy-2-oxoquinoxalin-1(2H)-yl)-N-(3,3-dimethylbutyl)-N-(5-hydroxypentyl)acetamide,
3-[[(3-tert-Butyl-7-methoxy-2-oxoquinoxalin-1(2H)-yl)acetyl](3,3-dimethylbutyl)amino]-2,2-dimethylpropyl dihydrogen phosphate,
2-[[(3-tert-Butyl-7-methoxy-2-oxoquinoxalin-1(2H)-yl)acetyl](3,3-dimethylbutyl)amino]-1,1-dimethylethyl dihydrogen phosphate,
3-tert-Butyl-1-(3,3-dimethyl-2-oxobutyl)-7-methoxyquinoxalin-2(1H)-one,
3-tert-Butyl-1-(3,3-dimethyl-2-oxopentyl)-7-methoxyquinoxalin-2(1H)-one,
3-tert-Butyl-1-(3-ethyl-3-methyl-2-oxopentyl)-7-methoxyquinoxalin-2(1H)-one,
3-tert-Butyl-7-methoxy-1-(3-methyl-2-oxobutyl)quinoxalin-2(1H)-one,
3-tert-Butyl-1-(2-cyclohexyl-2-oxoethyl)-7-methoxyquinoxalin-2(1H)-one,
3-tert-Butyl-1-(2-cyclopentyl-2-oxoethyl)-7-methoxyquinoxalin-2(1H)-one,
1-[2-(1-Adamantyl)-2-oxoethyl]-3-tert-butyl-7-methoxyquinoxalin-2(1H)-one,
2-(3-Isopropyl-7-methoxy-2-oxoquinoxalin-1(2H)-yl)-N,N-bis(3-methylbutyl)acetamide,
2-[3-[4-(3-Hydroxypropyl)phenyl]-7-methoxy-2-oxoquinoxalin-1(2R)-yl]-N,N-bis(3-methylbutyl)acetamide,
N-(3,3-Dimethylbutyl)-2-[3-[4-(3-hydroxypropyl)phenyl]-7-methoxy-2-oxoquinoxalin-1(2H)-yl]-N-propylacetamide,
3,3-Dimethyl-2-oxobutyl 4-[4-(3,3-dimethyl-2-oxobutyl)-6-methoxy-3-oxo-3,4-dihydroquinoxalin-2-yl]-4-methylpentanoate,
2-[3-(4-Hydroxy-1,1-dimethylbutyl)-7-methoxy-2-oxoquinoxalin-1(2H)-yl]-N,N-bis(3-methylbutyl)acetamide,
2-(3-tert-Butyl-7-methoxy-2-oxo-1,8-naphthyridin-1(2H)-yl)-N-(3,3-dimethylbutyl)-N-propylacetamide,
   or a pharmaceutically acceptable salt, ester including phosphate, enantiomer, diatereomer or mixture thereof.

This invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190)

When any variable (e.g. aryl, heterocycle, R₁, R₄ etc.) occurs more than one time in any constituent, its definition on each occurrence is independent at every other occurrence. Also, combinations of substituents/or variables are permissible only if such combinations result in stable compounds.

When R^{a} is —O— and attached to a carbon it is referred to as a carbonyl group and when it is attached to a nitrogen (e.g., nitrogen atom on a pyridyl group) or sulfur atom it is referred to a N-oxide and sulfoxide group, respectively.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopropyl cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, unless otherwise defined, without alternating or resonating double bonds between carbon atoms. It may contain from 1 to 4 rings, which can be fused. Examples of such cycloalkyl elements include, but are not limited to, cyclopropyl, cyclobutyl, cyclopentyl, cyclohexyl and cycloheptyl.

Alkenyl is C₂-C₆ alkenyl.

Alkoxy refers to an alkyl group of indicated number of carbon atoms attached through an oxygen bridge, with the alkyl group optionally substituted as described herein. Said groups are those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

Halogen (halo) refers to chlorine, fluorine, iodine or bromine.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Examples of aryl groups are phenyl, naphthyl, tetrahydronaphthyl, indanyl, biphenyl, phenanthryl, anthryl or acenaphthyl and phenanthrenyl, preferably phenyl, naphthyl or phenanthrenyl. Aryl groups may likewise be substituted as defined. Preferred substituted aryls include phenyl and naphthyl.

The term heterocyclyl or heterocyclic, as used herein, represents a stable 3- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, dihydropyrrolyl, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. Preferably, heterocycle is selected from 2-azepinonyl, benzimidazolyl, 2-diazapinonyl, dihydroimidazolyl, dihydropyrrolyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl. Additional nitrogen atoms may be present together with the first nitrogen and oxygen or sulfur, giving, e.g., thiadiazole.

In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

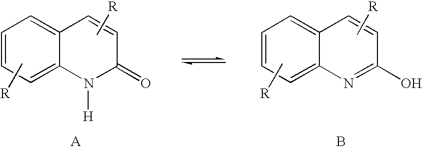

This invention is also concerned with compositions and methods of treating ocular hypertension or glaucoma by administering to a patient in need thereof one of the compounds of formula I in combination with one or more of a β-adrenergic blocking agent such as timolol, betaxolol, levobetaxolol, carteolol, levobunolol, a parasympathomimetic agent such as epinephrine, iopidine, brimonidine, clonidine, para-aminoclonidine, carbonic anhydrase inhibitor such as dorzolamide, acetazolamide, metazolamide or brinzolamide, an EP4 agonist (such as those disclosed in WO 02/24647, WO 02/42268, EP 1114816, WO 01/46140 and WO 01/72268), a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imidazol-6-ol fumarate and 2-(3-chloro-6-methoxy-indazol-1-yl)-1-methyl-ethylamine. An example of a hypotensive lipid (the carboxylic acid group on the □-chain link of the basic prostaglandin structure is replaced with electrochemically neutral substituents) is that in which the carboxylic acid group is replaced with a C₁₋₆ alkoxy group such as OCH₃ (PGF₂ₐ 1-OCH₃), or a ammalia group (PGF₂ₐ 1-OH).

Preferred potassium channel blockers are calcium activated potassium channel blockers. More preferred potassium channel blockers are high conductance, calcium activated potassium (Maxi-K) channel blockers. Maxi-K channels are a family of ion channels that are prevalent in neuronal, smooth muscle and epithelial tissues and which are gated by membrane potential and intracellular Ca²⁺.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering IOP promotes blood flow to the retina and optic nerve. Accordingly, the compounds of this invention are useful for treating macular edema and/or macular degeneration.

It is believed that maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof.

A number of marketed drugs function as potassium channel antagonists. The most important of these include the compounds Glyburide, Glipizide and Tolbutamide. These potassium channel antagonists are useful as antidiabetic agents. The compounds of this invention may be combined with one or more of these compounds to treat diabetes.

Potassium channel antagonists are also utilized as Class 3 antiarrhythmic agents and to treat acute infarctions in humans. A number of naturally ammalian toxins are known to block potassium channels including Apamin, Iberiotoxin, Charybdotoxin, Noxiustoxin, Kaliotoxin, Dendrotoxin(s), mast cell degranuating (MCD) peptide, and β-Bungarotoxin (β-BTX). The compounds of this invention may be combined with one or more of these compounds to treat arrhythmias.

Depression is related to a decrease in neurotransmitter release. Current treatments of depression include blockers of neurotransmitter uptake, and inhibitors of enzymes involved in neurotransmitter degradation which act to prolong the lifetime of neurotransmitters.

Alzheimer's disease is also characterized by a diminished neurotransmitter release. Three classes of drugs are being investigated for the treatment of Alzheimer's disease cholinergic potentiators such as the anticholinesterase drugs (e.g., physostigmine (eserine), and Tacrine (tetrahydroaminocridine)); nootropics that affect neuron metabolism with little effect elsewhere (e.g., Piracetam, Oxiracetam; and those drugs that affect brain vasculature such as a mixture of ergoloid mesylates amd calcium channel blocking drugs including Nimodipine Selegiline, a monoamine oxidase B inhibitor which increases brain dopamine and norepinephrine has reportedly caused mild improvement in some Alzheimer's patients. Aluminum chelating agents have been of interest to those who believe Alzheimer's disease is due to aluminum toxicity. Drugs that affect behavior, including neuroleptics, and anxiolytics have been employed. Anxiolytics, which are mild tranquilizers, are less effective than neuroleptics The present invention is related to novel compounds which are useful as potassium channel antagonists.

The compounds within the scope of the present invention exhibit potassium channel antagonist activity and thus are useful in disorders associated with potassium channel malfunction. A number of cognitive disorders such as Alzheimer's Disease, memory loss or depression may benefit from enhanced release of neurotransmitters such as serotonin, dopamine or acetylcholine and the like. Blockage of Maxi-K channels maintains cellular depolarization and therefore enhances secretion of these vital neurotransmitters.

The compounds of this invention may be combined with anticholinesterase drugs such as physostigmine (eserine) and Tacrine (tetrahydroaminocridine), nootropics such as Piracetam, Oxiracetam, ergoloid mesylates, selective calcium channel blockers such as Nimodipine, or monoamine oxidase B inhibitors such as Selegiline, in the treatment of Alzheimer's disease. The compounds of this invention may also be combined with Apamin, Iberiotoxin, Charybdotoxin, Noxiustoxin, Kaliotoxin, Dendrotoxin(s), mast cell degranuating (MCD) peptide, β-Bungarotoxin (O-BTX) or a combination thereof in treating arrythmias. The compounds of this invention may further be combined with Glyburide, Glipizide, Tolbutamide or a combination thereof to treat diabetes.

The herein examples illustrate but do not limit the claimed invention. Each of the claimed compounds are potassium channel antagonists and are thus useful in the described neurological disorders in which it is desirable to maintain the cell in a depolarized state to achieve maximal neurotransmitter release. The compounds produced in the present invention are readily combined with suitable and known pharmaceutically acceptable excipients to produce compositions which may be administered to mammals, including humans, to achieve effective potassium channel blockage.

For use in medicine, the salts of the compounds of formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted-amines including naturally occurring substituted amines, cyclic amines and basic ion exchange resins, such as arginine, betaine caffeine, choline, N,N¹-dibenzylethylenediamine, diethylamnin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

As used herein, the term “composition” is intended to encompass a product comprising the specified ingredients in the specific amounts, as well as any product which results, directly or indirectly, from combination of the specific ingredients in the specified amounts.

When a compound according to this invention is administered into a human subject, the daily dosage will normally be determined by the prescribing physician with the dosage generally varying according to the age, weight, sex and response of the individual patient, as well as the severity of the patient's symptoms.

The maxi-K channel blockers used can be administered in a therapeutically effective amount intravenously, subcutaneously, topically, transdermally, parenterally or any other method known to those skilled in the art.

Ophthalmic pharmaceutical compositions are preferably adapted for topical administration to the eye in the form of solutions, suspensions, ointments, creams or as a solid insert. Ophthalmic formulations of this compound may contain from 0.01 ppm to 1% and especially 0.1 ppm to 1% of medicament. Higher dosages as, for example, about 10% or lower dosages can be employed provided the dose is effective in reducing intraocular pressure, treating glaucoma, increasing blood flow velocity or oxygen tension. For a single dose, from between 1 ng to 500 ug, preferably 1 ng to 500 ug, of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the ammalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

Definitions of the terms used in the examples are as follows:

SM—Starting material,
DMSO—dimethyl sulfoxide,
TLC—thin layer chromatography,
SGC—silica gel chromatography,
PhMgBr—phenylmagnesiumbromide
h=hr=hour,
THF—tetrahydrofuran,
DMF—dimethylformamide,
min—minute,
LC/MS—liquid chromatography/mass spectrometry,
HPLC—high performance liquid chromatography,
PyBOP—Benzotriazol-1-yloxytris-pyrrolidino-phosphonium hexafluorophosphate, equiv=eq=equivalent,

### NBS—N-Bromosuccinamide,

AIBN—2,2′-azobisisobutyronitrile,

### DCM—Dichloromethane,

mCPBA—meta-Chloroperbenzoic acid,
TFA—Trifluoroacetic acid,

### DIEA—N,N-Diisopropylethylamine,

HOBt—1-Hydroxybenzotriazole hydrate,
EDC—N-(3-Dimethylaminopropyl)-N′-ethylcarbodiimide hydrochloride, and
HOAt—1-Hydroxy-7-azabenzotriazole.

The following examples given by way of illustration are demonstrative of the present invention.

Several methods are known in the literature for the preparation of the core structure of quinoli-2(1H)-one. For example, Turner (J. Org. Chem. 1990, 55, 4744) and Nielsen et al. (J. Amer. Chem. Soc. 2002, 124, 3254) have both published approaches to the core structure of quinoli-2(11)-one and some of the aza derivatives. The reaction sequence shown in Scheme 1 uses the method of Turner for the preparation of the quinoli-2(1H)-one core. The substituted benzaldehyde A was prepared by standard method. It was converted to substituted quinoli-2(1H)-one B using the method of Turner. Subsequent alkylation with bromo-ketones gave two isomeric products, which can be separated either on silica gel or using RP-HPLC. Analoguous alkylation of B using bromo-esters also afforded two isomeric products which can be separated using the same methods. Following hydrolysis of the esters to acid, standard amide formation reactions can be applied to provide amides.

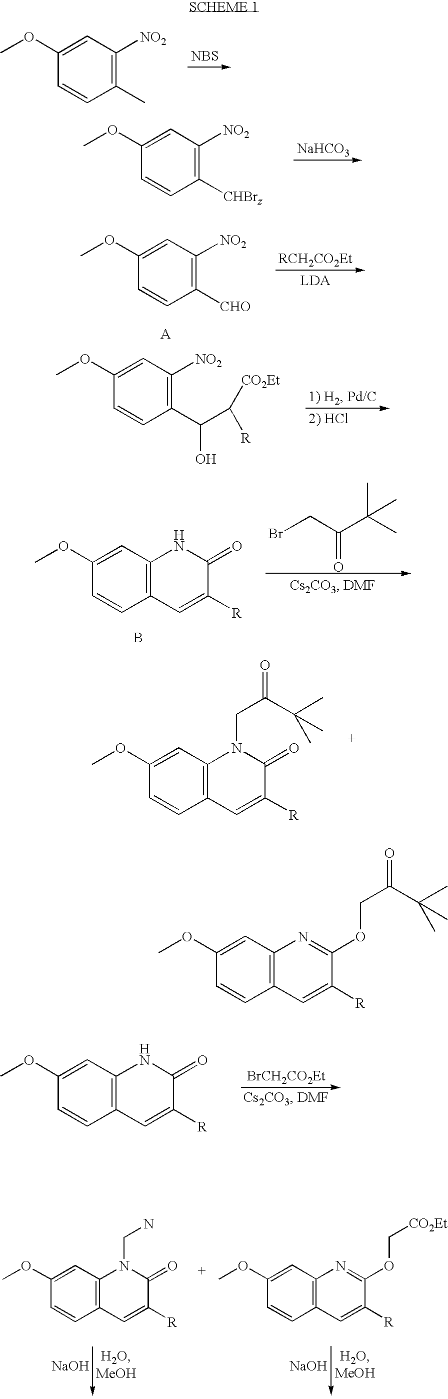

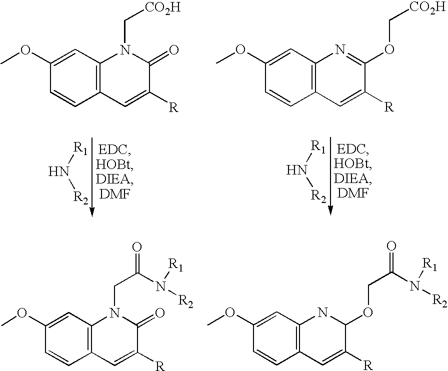

Two isomeric 4-aza-quinoli-2(1H)-ones can be synthesized by the reaction of an x-keto-acid with o-phenylenediamine (Scheme 2). The two isomers were separated on silica gel. Similar transformations as in Scheme 1 provide ketone or amide derivatives of interest.

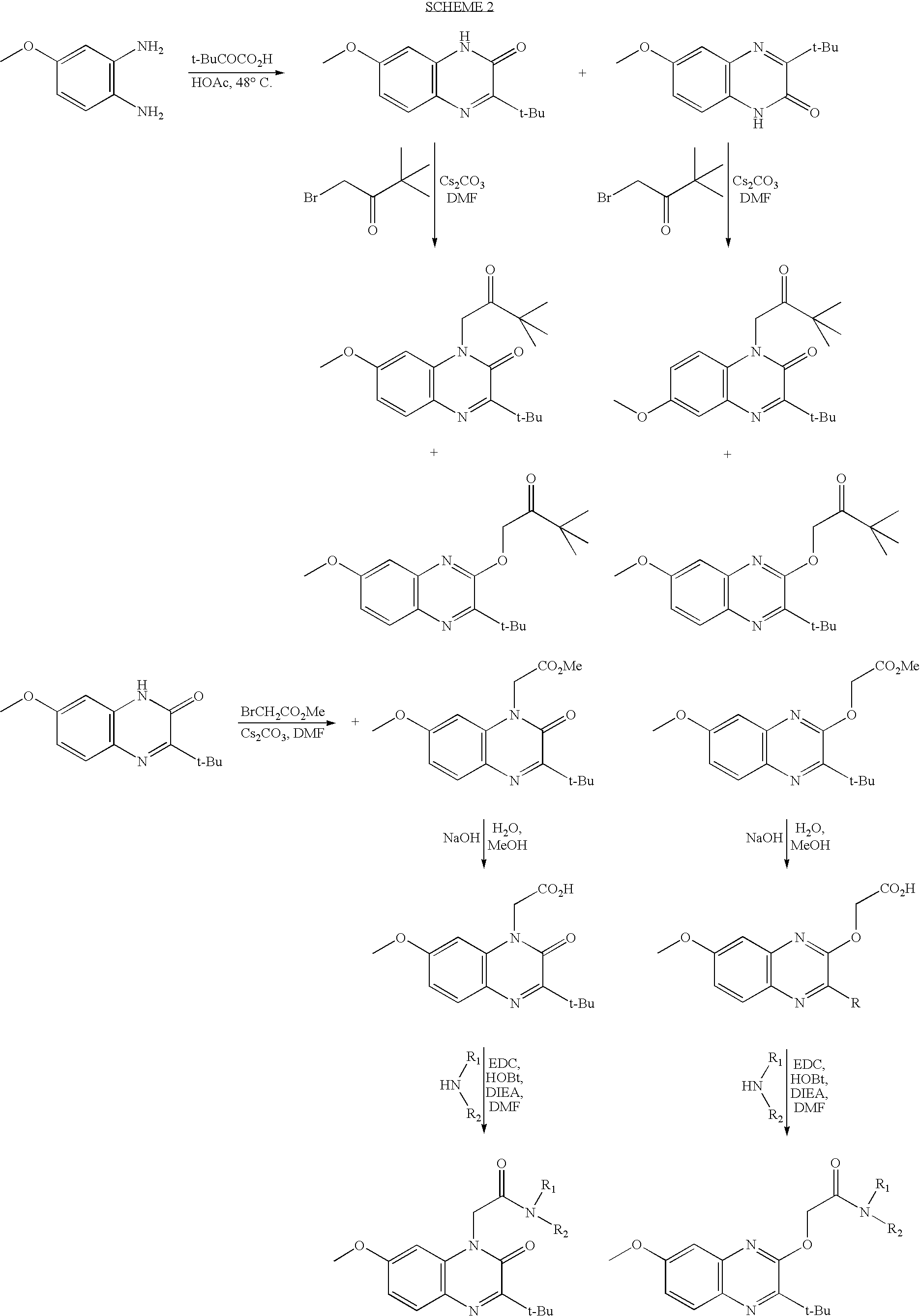

A similar method as in Scheme 2 can be used to prepare substituted pyrido[2,3-b]pyrazin-3(4H)-one (Scheme 3). An alternative method is shown in Scheme 4.

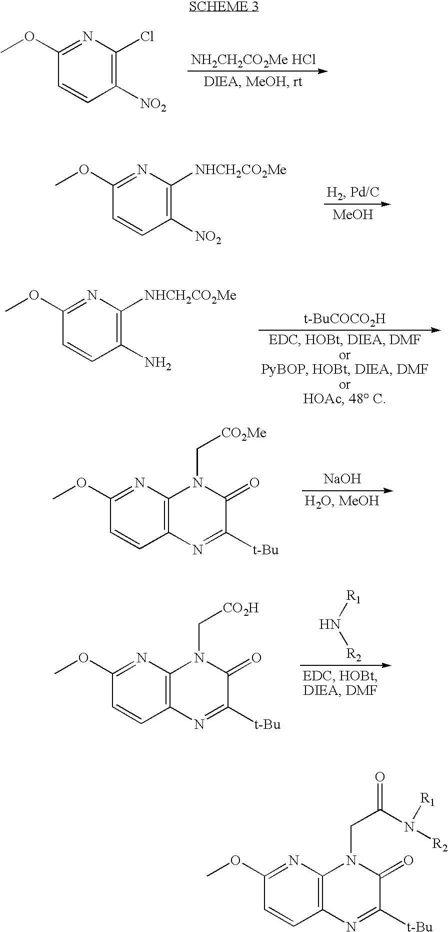

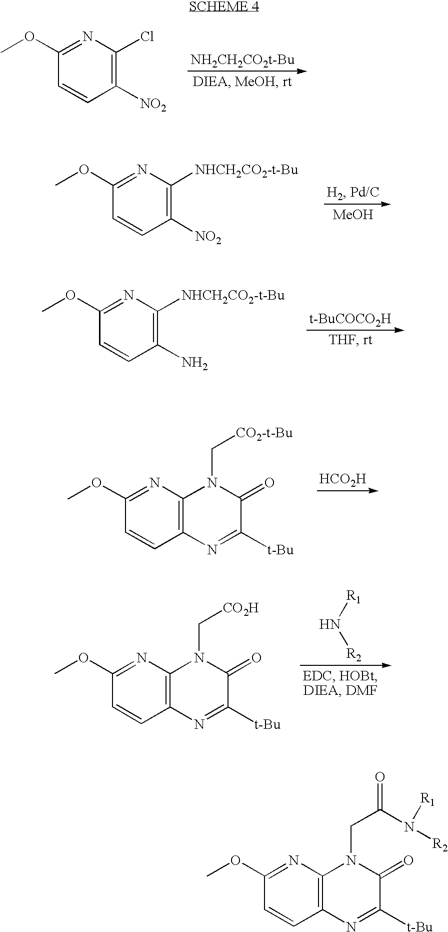

Using the modified method of Turner, the core heterocycle of 1,8-naphthyridin-2(1H)-one can be prepared as shown in Scheme 5. Substituted analogues could be prepared from a more elaborated starting material or obtained by further transformation of the un-substituted heterocycle using the method depicted in Schemes 6 and 7.

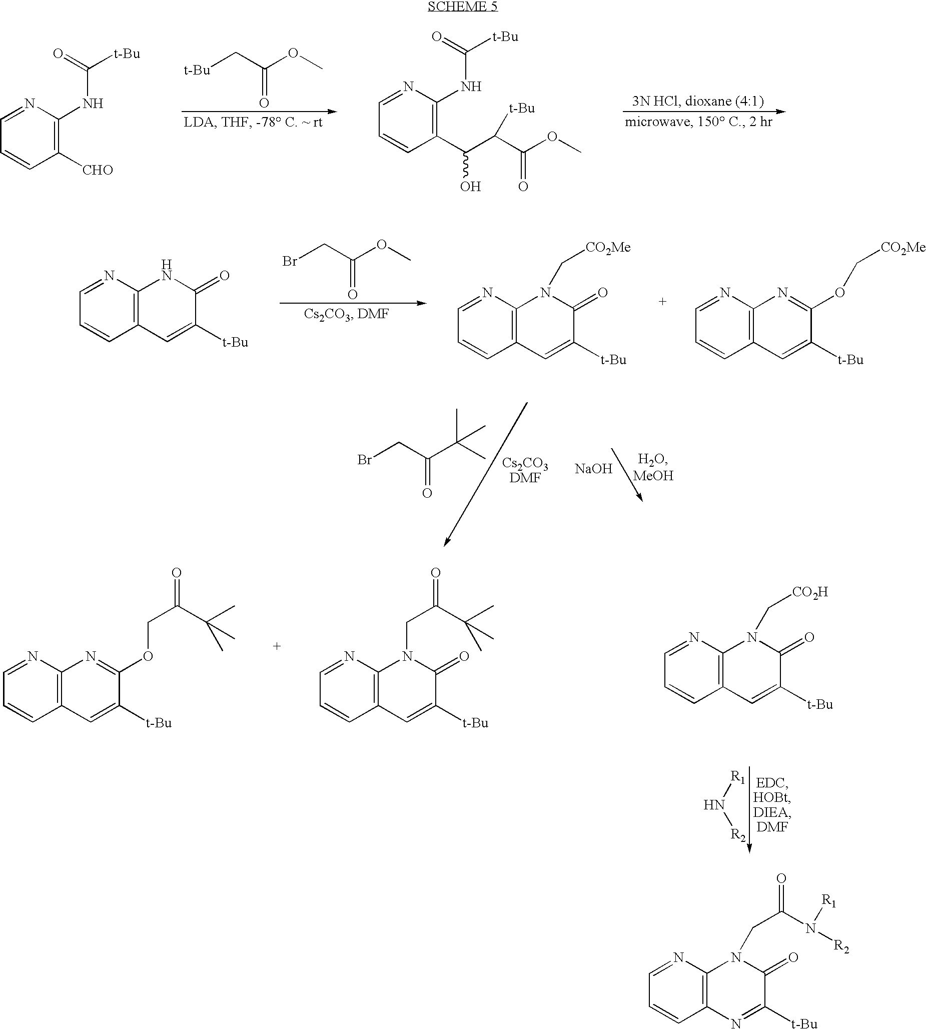

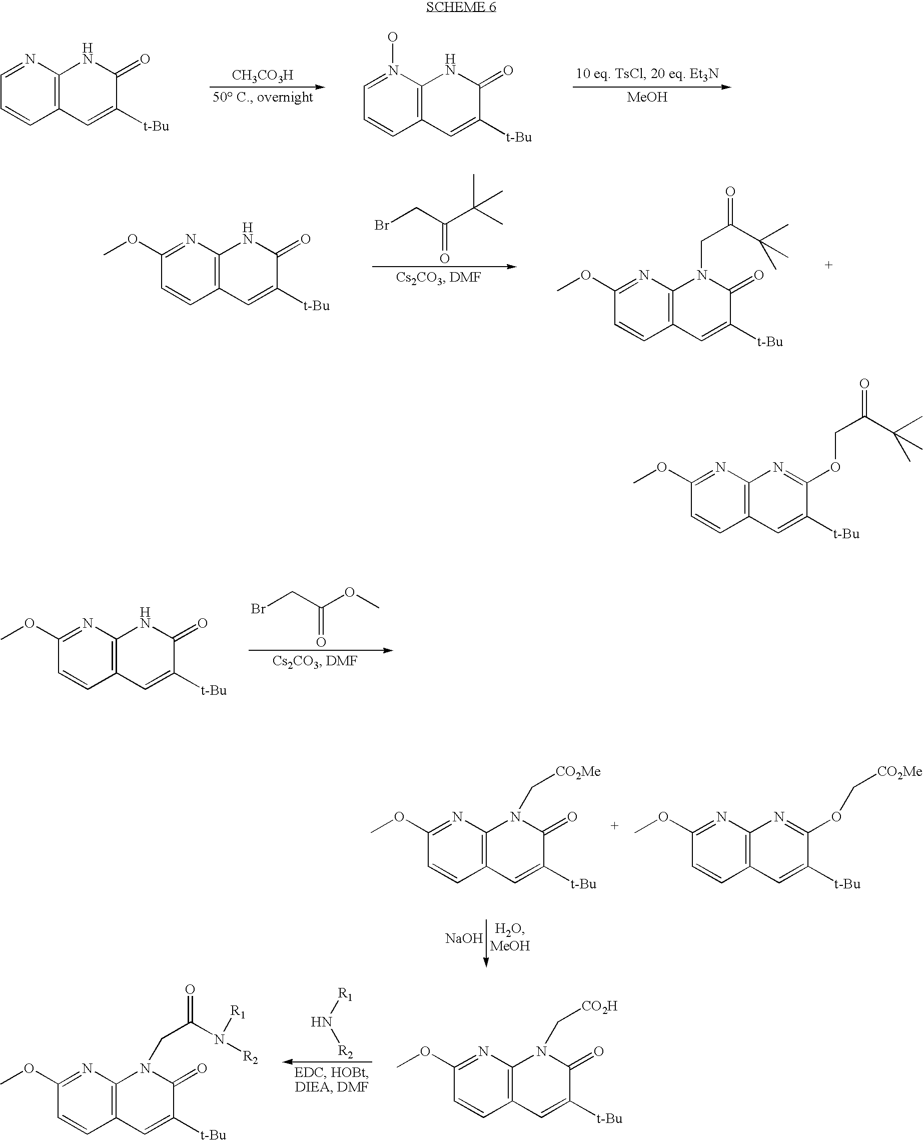

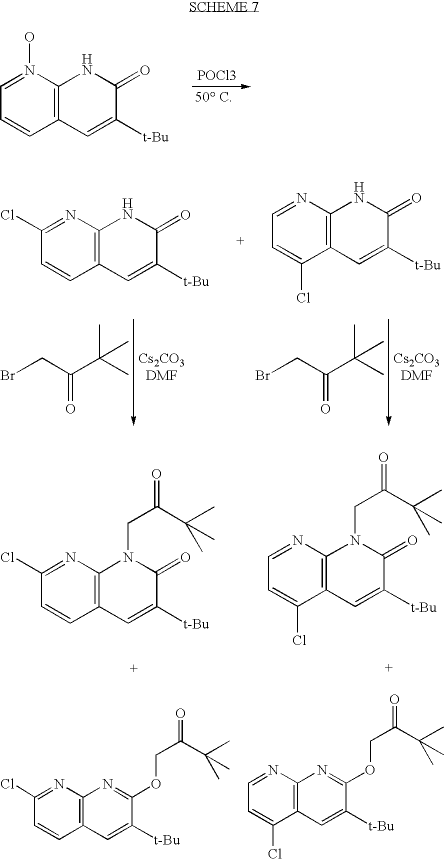

Similarly, the core heterocycle of 1,6-naphthyridin-2(1H)-one 1 can be prepared as shown in Scheme 8. Substituted analogues could be prepared from a more elaborated starting material or obtained by further transformation of the un-substituted heterocycle using the method depicted in Scheme 9.

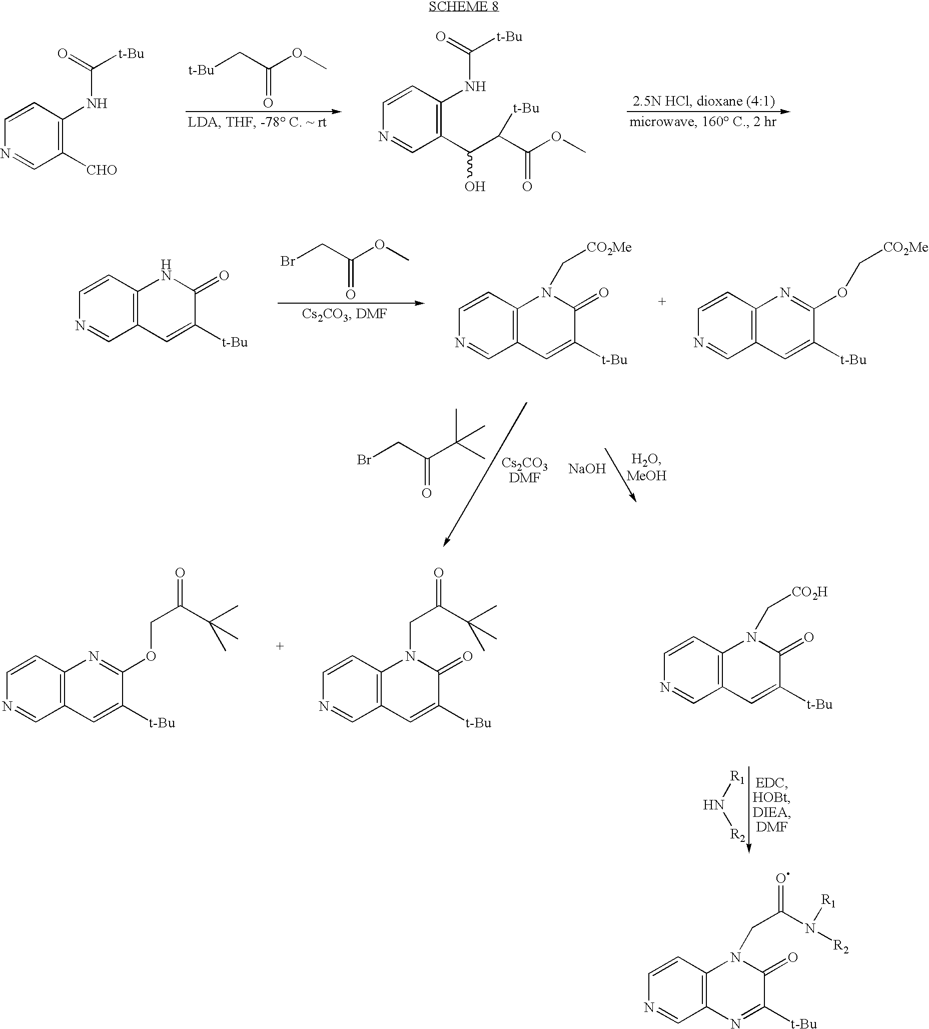

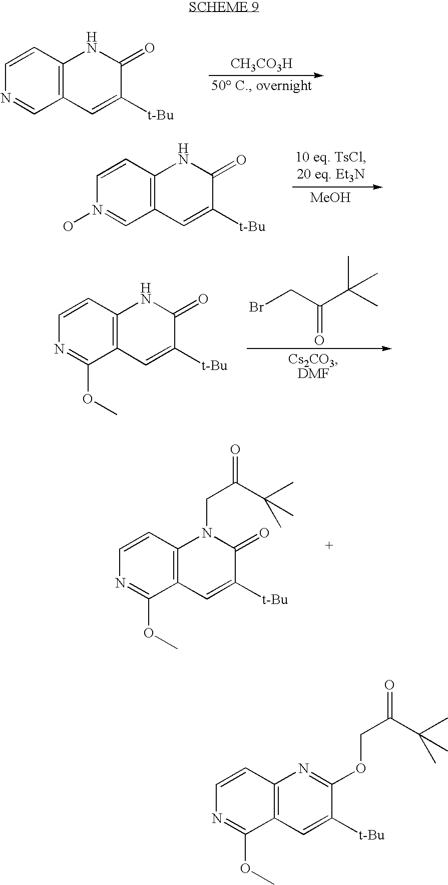

Preparation of phosphates of hydroxyl amides are illustrated in Schemes 10 and 11.

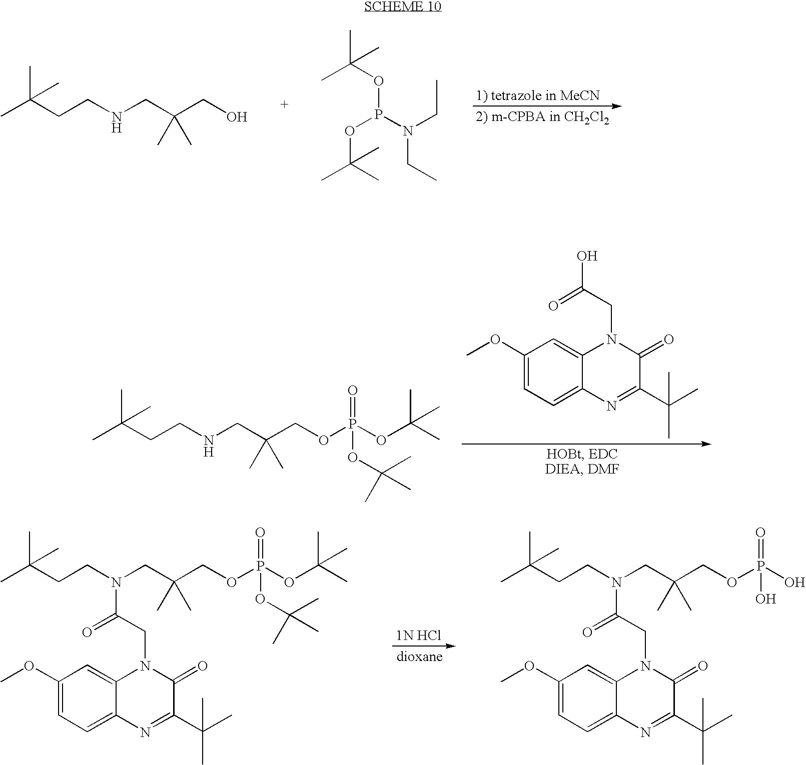

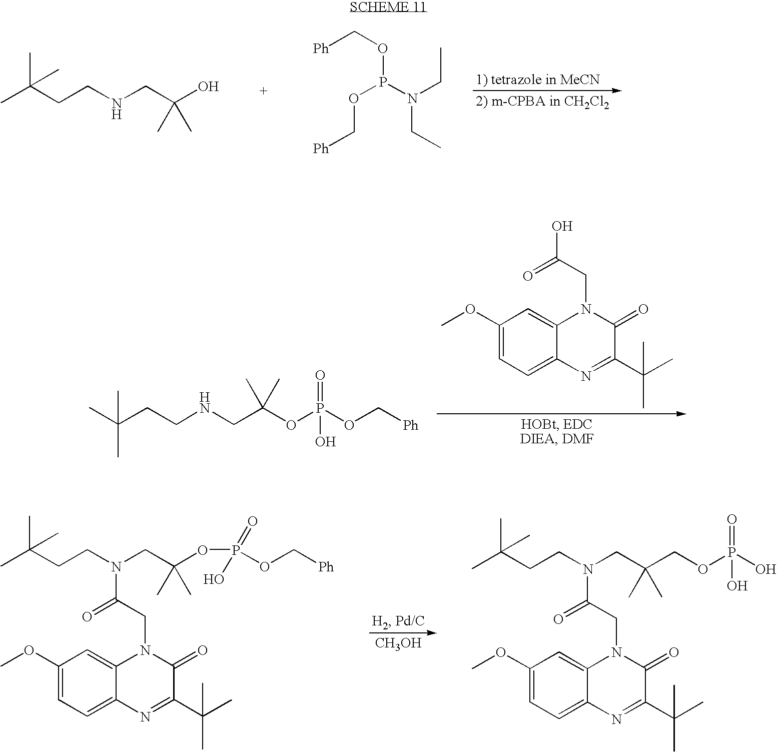

### PREPARATIVE EXAMPLE 1

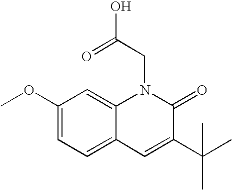

### (3-Tert-butyl-7-methoxy-2-oxoquinolin-1(2H)-yl)acetic acid

### Step A. 1-(Dibromomethyl)-4-methoxy-2-nitrobenzene

The title compound was prepared from 4-methoxy-1-methyl-2-nitrobenzene using the method of Suvorov, N. N.; et al.; J Gen Chem USSR (Engl Transl) 1960, 30, 3118 and purified using silica gel column with 100:7 hexanes and ethyl acetate. ¹H NMR in CDCl₃ at 500 MHz: 7.57 (d, 2.5 Hz, 1H), 7.48 (d, 9.0 Hz, 1H), 7.15 (dd, 9 Hz, 2.5 Hz, 1M, 4.82 (s, 2H), 3.91 (s, 3H).

### Step B: 4-Methoxy-2-nitrobenzaldehyde

The title compound was prepared from 1-(dibromomethyl)-4-methoxy-2-nitrobenzene using the method of Suvorov, N. N.; et al.; J Gen Chem USSR (Engl Transl) 1960, 30, 3118 and purified using silica gel column with 5:1 hexanes and ethyl acetate followed by methylene chloride. ¹H NMR in CDCl₃ at 500 MHz: 10.32 (s, 1H), 8.01 (d, 8.5 Hz, 1H), 7.54 (d, 2.5 Hz, 1H), 7.26 (dd, 8.5 Hz, 2.5 Hz, 1H), 3.99 (s, 3H).

### Step C: Ethyl 2-[hydroxy(4-methoxy-2-nitrophenyl)methyl]-3,3-dimethylbutanoate

A 2 M solution of LDA in heptane/THF/ethylbenzene (15.2 mL) was added to a mixture of 4.0 g ethyl 3,3-dimethylbutanoate in 100 mL anhydrous THF at −78° C. 4-methoxy-2-nitrobenzaldehyde (5.0 g) from the step above was added to the reaction mixture. The resulting mixture was stirred for 2 hours and was allowed to warm to room temperature. The reaction was quenched by addition of 2 mL water. Solvents were removed under reduced pressure and the residue was partitioned between EtOAc and water. The organic extract was washed with 1 N HCl and NaHCO₃. The resulting crude product was purified on silica gel using 100:15 hexanes and EtOAc to give two diastereomeric isomers of the title compound. ¹H NMR in CDCl₃ at 500 MHz of the less polar pair of isomers: 7.56 (d, 9 Hz, 1H), 7.52 (d, 2.5 Hz, 1H), 7.16 (dd, 9 Hz, 2.5 Hz, 1H), 5.73 (d, 8.5 Hz, 1H), 4.84 (d, 9.5 Hz, 1H), 4.10-3.95 (m, 2H), 3.88 (s, 3H), 2.64 (d, 2 Hz, 1H), 1.20 (s, 9H), 1.09 (t, 7 Hz, 3H)

### Step D: Ethyl 2-[(2-amino-4-methoxyphenyl)(hydroxy)methyl]-3,3-dimethylbutanoate

A solution of 2.5 g ethyl 2-[hydroxy(4-methoxy-2-nitrophenyl)methyl]-3,3-dimethylbutanoate from the step above in 100 mL ethanol was treated with a catalytic amount of 10% Pd/C and a hydrogen balloon overnight. The reaction mixture was then filtered and concentrated under reduced pressure to give title compound.

### Step E: 3-Tert-butylquinolin-2(1H)-one

A mixture of 1.5 g ethyl 2-[(2-amino-4-methoxyphenyl)(hydroxy)methyl]-3,3-dimethylbutanoate from the step above with 35 mL concentrated HCl was refluxed for 2 hours. After cooling, the precipitate was collected by filtration, washed with concentrated HCl, and dried to give the title compound as a white solid. LC-MS: 3.30 min. (m/Z 232.4). ¹H NMR in CDCl₃ at 500 MHz. 7.64 (s, 1H), 7.44 (d, 9 Hz, 1H), 6.85-6.84 (m, 1H), 6.80 (dd, 9 Hz, 2.5 Hz, 1H), 3.90 (s, 3H), 1.50 (s, 9H).

### Step F: Ethyl (3-tert-butyl-2-oxoquinolin-1(2H)-yl)acetate

To a mixture of 1.0 g 3-tert-butylquinolin-2(1H)-one from the step above, 2.80 g cesium carbonate, and 5 mL dry DMF was added 0.73 mL (6.5 mmole) ethyl bromoacetate. The reaction mixture was stirred for 8 hours at room temperature. Aqueous EtOAc work-up afforded a crude product which was purified on silica gel. Elution with 100:5 hexanes and EtOAc gave 250 mg of the side-product ethyl [(3-tert-butylquinolin-2-yl)oxy]acetate as a sticky oil. Further elution with 100:15 hexanes and EtOAc gave 950 mg of the title compound as a solid. ¹H NMR in CD₃OD at 500 MHz of the more polar major product: 7.73 (s, 1H), 7.57 (d, 8.4 Hz, 1H), 6.87 (dd, 1.9 & 8.6 Hz, 1H), 6.71 (d, 1.8 Hz, 1H), 5.08 (s, 2H), 4.23 (q, 7.1 Hz, 2H), 3.86 (s, 3H), 1.40 (s, 9H), 1.27 (t, 7.1 Hz, 3H). NOE difference spectrum from irradiation of 5.08 singlet gave a large NOE at 6.71 doublet. ¹H NMR in CD₃OD at 500 MHz of the less polar minor product: 7.943 (s, 1H), 7.65 (d, 8.9 Hz, 1H), 7.09 (d, 2.5 Hz, 1H), 7.00 (dd, 2.6 & 8.8 Hz, 1H), 5.06 (s, 2H), 4.21 (q, 7.1 Hz, 2H), 3.88 (s, 3H), 1.48 (s, 9H), 1.23 (t, 7.1 Hz, 3H). NOE difference spectrum from irradiation of 5.06 singlet showed no significant NOE anywhere.

### Step G: (3-Tert-butyl-7-methoxy-2-oxoquinolin-1(2H)-yl)acetic acid

A mixture of 1.0 g ethyl (3-tert-butyl-2-oxoquinolin-1(2H)-yl)acetate from the step above, 1.32 g lithium hydroxide monohydrate, and 20 mL 1:1 dioxane and water was stirred at room temperature overnight. The organic solvent was removed under reduced pressure and the resulting mixture was acidified to pH 1 using concentrated HCl. The mixture was partitioned between water and EtOAc. The title compound was obtained from the organic layer as a white solid. LC-MS: 3.20 min. (m/Z 290.25). ¹H NMR in DMSO-d₆ at 500 MHz: 7.71 (s, 1H), 7.65 (d, 8.5 Hz, 1H), 6.65 (dd, 9 Hz, 2 Hz, 1H), 6.76 (d, 2 Hz, 1H), 4.97 (s, 2H), 3.82 (s, 3H), 1.33 (s, 9H).

### PREPARATIVE EXAMPLE 2

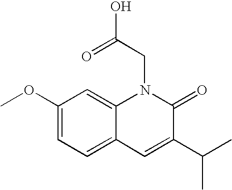

### (3-Isopropyl-2-oxoquinolin-1(2H)-yl)acetic acid

The title compound was prepared using the procedure described in Steps C˜G of Preparative Example 1 and starting with methyl 3-methylbutanoate and 4-methoxy-2-nitrobenzaldehyde. LC-MS: 3.15 min. (m/Z 217.1). ¹H NMR in DMSO-d₆ at 500 MHz: 7.69 (s, 1H), 7.63 (d, 8.5 Hz, 1H), 6.87 (dd, 8.5 Hz, 2 Hz, 1H), 6.79 (d, 2 Hz, 1H), 5.00 (s, 2H), 3.83 (s, 3H), 3.09-3.04 (m, 1H), 1.16 (d, 7 Hz, 6H).

### PREPARATIVE EXAMPLE 3

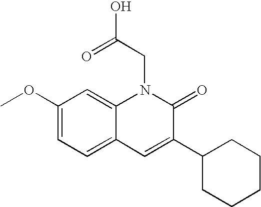

### (3-Cyclohexyl-2-oxoquinolin-1(2H)-yl)acetic acid

The title compound was prepared using the procedure described in Steps C˜G of Preparative Example 1 and starting with methyl 3-methylbutanoate and 4-methoxy-2-nitrobenzaldehyde. ¹H NMR in DMSO-d₆ at 500 MHz: 7.65 (s, 1H), 7.62 (d, 9 Hz, 1H), 6.86 (dd, 9 Hz, 2.5 Hz, 1H), 6.78 (d, 2 Hz, 1H), 4.98 (s, 2H), 3.82 (s, 3H), 2.75-2.71 (m, 1H), 3.09-3.04 (m, 1H), 1.82-1.69 (m, 5H), 1.39-1.18 (m, 5H).

### PREPARATIVE EXAMPLE 4

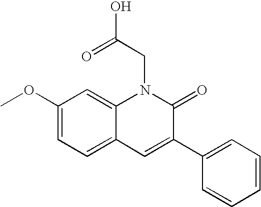

### (2-Oxo-3-phenylquinolin-1(2H)-yl)acetic acid

The title compound was prepared using the procedure described in Steps C˜G of Preparative Example 1 and starting with methyl phenyl acetate and 4-methoxy-2-nitrobenzaldehyde. ¹H NMR in DMSO-d₆ at 500 MHz: 8.09 (s, 1H), 7.74 (d, 9 Hz, 1H), 7.70 (d, 7.5 Hz, 2H), 7.41 (t, 7.5 Hz, 2H), 7.34 (t, 7.5 Hz, 1H), 6.93 (dd, 8.5 Hz, 2 Hz, 1H), 6.89 (s, 1H), 5.06 (s, 2H), 3.87 (s, 3H).

### PREPARATIVE EXAMPLE 5

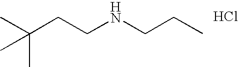

### 3,3-Dimethyl-N-propylbutan-1-amine hydrochloride

A mixture of 25 mL 3,3-dimethylbutyradehyde, 11.77 g (16.4 mL) n-propylamine, and 20 g dry 10˜16 mesh 4 Å molecular sieve in 200 mL hexanes was heated at 60° C. for 10 hours. The reaction mixture was filtered and the filtrate was concentrated under vacuum to give sticky oil. The latter was treated with 40 psi hydrogen with catalytic amount of 10% Pd/C in EtOAc for 4 hours. The reaction mixture was filtered. The concentrated filtrate was treated with 1 M HCl/ether to give title compound as a white solid. LC-MS: 2.31 min. (m/Z 144.7). ¹H NMR in DMSO-d₆ at 500 MHz: 2.98-2.89 (m, 4H), 1.99-1.94 (m, 2H), 1.86-1.82 (m, 2H), 1.25 (t, 3.5 HZ 3H), 0.96 (s, 9H).

### PREPARATIVE EXAMPLE 6

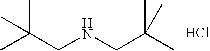

### N-(2,2-dimethylpropyl)-2,2-dimethylpropan-1-amine hydrochloride

The title compound was prepared using the procedure described in Preparative Example 5 and starting with trimethyl acetaldehyde and 2,2-dimethylpropylamine. LC-MS: 1.95 min. (m/Z 158.2). ¹H NMR in DMSO-d₆ at 500 MHz: 2.98-2.96 (m, 4H), 1.02 (s, 18H).

### PREPARATIVE EXAMPLE 7

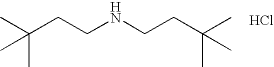

### N-(3,3-dimethylbutyl)-3,3-dimethylbutan-1-amine hydrochloride

The title compound was prepared using the procedure described in Preparative Example 5 and starting with trimethyl acetaldehyde and 2,2-dimethylpropylamine. LC-MS: 2.74 min. (m/Z 186.3). ¹H NMR in DMSO-d₆ at 500 MHz: 2.64-2.61 (m, 4H), 1.43-1.40 (m, 4H), 0.92 (s, 18H).

### PREPARATIVE EXAMPLE 8

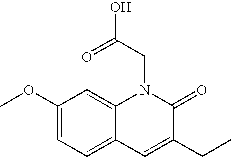

### (3-Ethyl-7-methoxy-2-oxoquinolin-1(2H)-yl)acetic acid

The title compound was prepared using the procedure described in Steps C˜G of Preparative Example 1 and starting with methyl n-butanoate and 4-methoxy-2-nitrobenzaldehyde. ¹H NMR in DMSO-d₆ at 500 MHz: 7.69 (s, 1H), 7.60 (dd, 8.5 & 2.0 Hz, 1H), 6.88 (dd, 8.5 & 2.0 Hz, 1H), 6.80 (s, 1H), 5.01 (s, 2H), 3.83 (s, 3H), 2.48 (q, 7.5H, 2H), 1.47 (t, 7.5 Hz, 3H).

### PREPARATIVE EXAMPLE 9

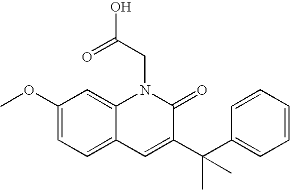

### [7-Methoxy-3-(1-methyl-1-phenylethyl)-2-oxoquinolin-1(2H)-yl]acetic acid

### Step A. 3-Methyl-3-phenylbutanoic acid

A 0.5 M ether solution of 2-methyl-2-phenylpropylmagnesium chloride (795 mL) was cooled to −78° C. and treated with carbon dioxide gas. The reaction mixture was slowly warmed up to room temperature. Work-up with EtOAc and aqueous HCl gave the title compound as sticky oil. ¹H NMR in CDCl₃ at 500 MHz: 7.41 (d, 8.0 Hz, 2H), 7.36 (t, 8.0 Hz, 2H), 7.24 (dt, 7.5 & 1.0 Hz, 1H), 2.69 (s, 2H), 1.51 (s, 6H).

### Step B. Methyl 3-methyl-3-phenylbutanoate

3-Methyl-3-phenylbutanoic acid (33 g) from the Step A above and 300 g cesium carbonate was refluxed in 450 mL 3:1 THF and DMF. Methyl iodide (57 mL) was added slowly over 5 hours. The resulting mixture was refluxed for 10 hours. Aqueous EtOAc work-up and silica gel column purification using 10:1 hexanes and EtOAc afforded the title compound as colorless oil. ¹H NMR in CDCl₃ at 500 MHz: 7.41 (d, 8.0 Hz, 2H), 7.34 (t, 8.0 Hz, 2H), 7.23 (dt, 7.5 & 1.0 Hz, 1H), 3.56 (s, 3H), 2.66 (s, 2H), 1.49 (s, 6H).

### Steps C˜G. [7-Methoxy-3-(1-methyl-1-phenylethyl)-2-oxoquinolin-1(2H)-yl]acetic acid

The title compound was prepared using the procedure described in Steps C˜G of Preparative Example 1 and starting with methyl 3-methyl-3-phenylbutanoate and 4-methoxy-2-nitrobenzaldehyde. LC-MS: 3.32 min. (m/Z 352.1). ¹H NMR in DMSO-d₆ at 500 MHz: 7.93 (s, 1H), 7.74 (d, 8.5 Hz, 1H), 7.20-7.16 (m, 4H), 7.09-7.06 (m, 1H), 6.90 (dd, 8.5 & 2.0 Hz, 1H), 7.62 (d, 2.0 Hz, 1H), 4.85 (s, 2H), 3.84 (s, 3H), 1.62 (s, 6H).

### PREPARATIVE EXAMPLE 10

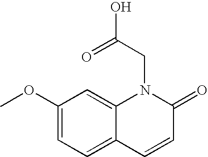

### (7-Methoxy-2-oxoquinolin-1(2H)-yl)acetic acid

### Step A. 7-Methoxyquinolin-2(1H)-one

The title compound as isolated as a side-product from Step E of Preparative Example 9. LC-MS: 2.07 min. (m/Z 176.1). ¹H NMR in DMSO-d₆ at 500 MHz: 7.79 (d, 9.5 Hz, 1H), 7.54 (d, 8.5 Hz, 1H), 6.79-6.76 (m, 2H), 6.28 (d, 9.5 Hz, 1H), 3.79 (s, 3H).

### Steps B and C. (7-Methoxy-2-oxoquinolin-1(2H)-yl)acetic acid

The title compound was prepared using the procedure described in Steps F˜G of Preparative Example 1 and starting with 7-methoxyquinolin-2(1H)-one from the Step A above. 1H NMR in DMSO-d₆ at 500 MHz: 7.87 (d, 9.5 Hz, 1H), 7.65 (d, 8.5 Hz, 1H), 6.90 (dd, 8.0 Hz, 2.0 Hz, 1H), 6.83 (d, 2.0 Hz, 1H), 6.43 (d, 9.0 Hz, 1H), 4.99 (s, 2H), 3.84 (s, 3H).

### PREPARATIVE EXAMPLE 11

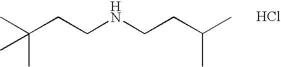

### 3,3-Dimethyl-N-(3-methylbutyl)butan-1-amine hydrochloride

The title compound was prepared using the procedure described in Preparative Example 5 and starting with 3-methylbutyraldehyde and 3,3-dimethylbutylamine. LC-MS: 2.40 min. (m/Z 172.2). ¹H NMR in CDCl₃ at 500 MHz: 2.91-2.88 (m, 4H), 1.79-1.73 (m, 4H), 1.74-1.66 (m, 1H), 0.95 (s, 9H), 0.94 (s, 6H).

### PREPARATIVE EXAMPLE 12

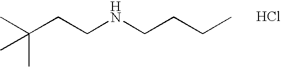

### N-Butyl-3,3-dimethylbutan-1-amine hydrochloride

N-butyl-3,3-dimethylbutanamide was prepared from n-butylamine, t-butylacetyl chloride, and DIEA. The crude amide was reduced in refluxing benzene with 1.5 molar equiv. of LAH in 2 hours. The excess LAH was quenched with MeOH and 1 N KOH after cooling. The resulting mixture was filtered and the solid washed with ether. The residue from the organic layer was dissolved in ether and treated with 1 N HCl in ether to precipitate the title compound. ¹H NMR in CD₃OD at 500 MHz: 3.03-2.99 (m, 4H), 1.70-1.64 (m, 2H), 1.62-1.58 (m, 2H), 1.47-1.40 (m, 2H), 0.99 (t, 4.5 Hz, 3H), 0.98 (s, 9H).

### PREPARATIVE EXAMPLES 13˜26

The following compounds in Table 1 were prepared using the method described in Preparative Example 12. For Preparative Examples 27 the LAH reduction were done in refluxing THF. For Preparative Examples 28˜29, they were conducted in boiling dioxane.

TABLE 1
Preparation of amines

| Preparative | | | LC-MS, |
|---|---|---|---|
| Example | Compound | | min. (m/Z) |
| 13 | 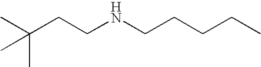 | HCl | — |
| 14 | 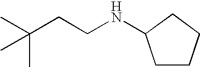 | HCl | — |
| 15 | 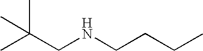 | HCl | — |
| 16 | 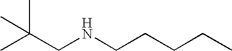 | HCl | — |
| 17 | 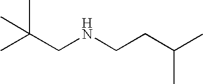 | HCl | — |
| 18 | 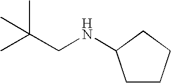 | HCl | 0.36 (156.1) |
| 19 | 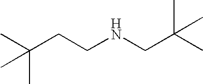 | HCl | — |
| 20 | 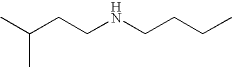 | HCl | 1.80 (144.1) |
| 21 | 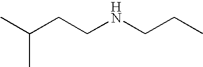 | HCl | 0.37 (130.1) |
| 22 | 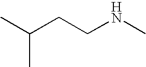 | HCl | — |
| 23 | 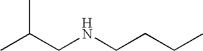 | HCl | — |
| 24 | 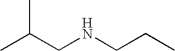 | HCl | — |
| 25 | 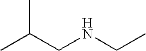 | HCl | — |
| 26 | 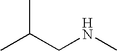 | HCl | — |
| 27 | 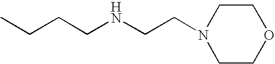 | | 0.37 (113.9,187.2) |
| 28 | 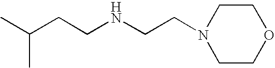 | | 0.49 (201.3) |
| 29 | 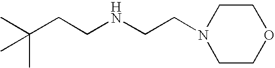 | | 0.81 (215.3) |
| 30 | 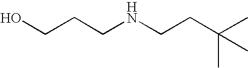 | | — |
| 31 | 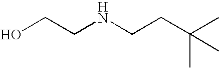 | | — |
| 32 | 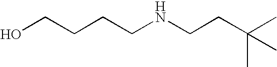 | | — |
| 33 | 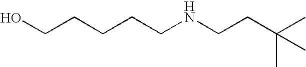 | | — |
| 34 | 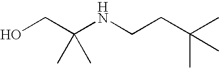 | | — |

### PREPARATIVE EXAMPLE 35

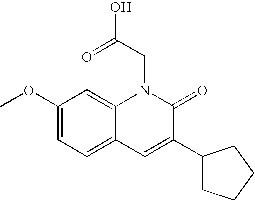

### (3-Cyclopentyl-7-methoxy-2-oxoquinolin-1(2H)-yl)acetic acid

The title compound was prepared using the procedure described in Steps C˜G of Preparative Example 1 and starting with methyl cyclopentylacetate and 4-methoxy-2-nitrobenzaldehyde. ¹H NMR in DMSO-d₆ at 500 MHz: 7.71 (s, 1H), 7.62 (d, 9.0 Hz, 1H), 6.87 (dd, 8.5 & 1.5 Hz, 1H), 6.78 (d, 1.5 Hz, 1H), 5.00 (s, 2H), 3.82 (s, 3H), 3.11 (m, 1H), 1.93 (m, 2H), 1.73 (m, 2H), 1.62 (m, 2H), 1.53 (m, 2H).

### PREPARATIVE EXAMPLE 36

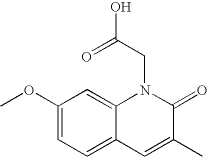

### (7-Methoxy-3-methyl-2-oxoquinolin-1(2H)-yl)acetic acid

The title compound was prepared using the procedure described in Steps C˜G of Preparative Example 1 and starting with ethyl propionate and 4-methoxy-2-nitrobenzaldehyde. LC-MS: 2.44 min. (m/Z 248.1). ¹H NMR in DMSO-d₆ at 500 MHz: 7.74 (s, 1H), 7.56 (d, 8.5 Hz, 1H), 6.88 (dd, 8.5 Hz, 2.0 Hz, 1H), 6.80 (d, 2.0 Hz, 1H), 5.00 (s, 2H), 3.83 (s, 3H), 2.07 (s, 3H).

### PREPARATIVE EXAMPLE 37

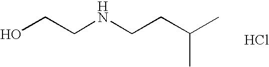

### 2-[(3-Methylbutyl)amino]ethanol hydrochloride

To a mixture of 10 g N-isovaleroylglycine in 200 mL dry THF at 0° C. was added 200 mL 1 M LAH in THF. The resulting mixture was refluxed for 10 hrs. The reaction mixture was cooled, quenched, filtered, and concentrated under vacuum. The residue was treated with 1 M HCl in ether, washed with 1:1 hexanes and ether, and dried to give the title compound as a white solid. ¹H NMR in CDCl₃ at 500 MHz: 3.66 (t, 5.0 Hz, 2H), 2.79 (t, 5.0 Hz, 2H), 2.65 (t, 7.5 Hz, 2H), 1.65 (m, 1H), 1.40 (m, 2H), 0.92 (d, 6.0 Hz, 6H).

### PREPARATIVE EXAMPLE 38

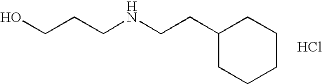

### 3-[(2-Cyclohexylethyl)amino]propan-1-ol hydrochloride

Cyclohexylacetic acid (20 g) was coupled with 13.7 g 3-hydroxypropylamine using 54 g EDC in the presence of 28.5 g HOBt and 86 mL DIEA in 200 mL dry DMF at room temperature. The reaction mixture was worked up using aqueous EtOAc. The organic extract was washed with 1 N HCl and NaHCO₃. The crude white solid (7.38 g) was dissolved in benzene and treated with 74 mL 1 M LAH at 0° C. followed by refluxing for 10 hours. Usual work-up afforded the crude amine which was precipitated from ether with 1 N HCl in ether to give the title compound. LC-MS: 1.76 min. (m/Z 186.3). ¹H NMR in CDCl₃ at 500 MHz: 3.82 (t, 5.5 Hz, 2H), 3.11 (br s, 2H), 2.89 (t, 6.0 Hz, 2H), 2.64 (t, 7.5 Hz, 2H), 1.73-1.64 (m, 7H), 1.38 (m, 2H), 1.33-1.11 (m, 4H), 0.90 (m, 2H).

### PREPARATIVE EXAMPLE 39

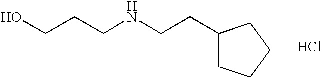

3-[(2-Cyclopentylethyl)amino]propan-1-ol hydrochloride

The title compound was prepared using the same method as described in Preparative Example 38. LC-MS: 1.56 min. (m/Z 172.2). ¹H NMR in CDCl₃ at 500 MHz: 3.82 (t, 5.5 Hz, 2H), 2.89 (t, 5.5 Hz, 2H), 2.63 (t, 7.5 Hz, 2H), 1.81-1.73 (m, 3H), 1.70 (m, 2H), 1.61 (m, 2H), 1.450 (m, 4H), 1.10 (m, 2H).

### PREPARATIVE EXAMPLE 40

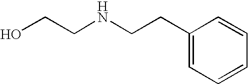

### 2-[(2-Phenylethyl)amino]ethanol

The title compound was prepared using the same method as described in Preparative Example 37 from phenaceturic acid. LC-MS: 0.67 min. (m/Z 166.7).

### PREPARATIVE EXAMPLE 41

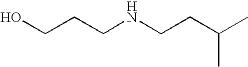

### 3-[(3-Methylbutyl)amino]propan-1-ol

The title compound was prepared by heating 30 g 1-iodo-3-methylbutane and 13.7 g 3-aminopropan-1-ol in 200 mL DMF at 100° C. in the presence of 65 g potassium carbonate for 12 hrs. Work-up and evaporation under vacuum gave the title compound as yellow oil. LC-MS: 0.34 min. (m/Z 146.1). ¹H NMR in CDCl₃ at 500 MHz: 3.81 (q, 6.0 Hz, 2H), 2.64 (m, 2H), 2.44 (m, 2H), 1.70 (m, 2H), 1.56 (m, 1H), 1.37 (m, 2H), 0.91 (d, 6.0 Hz, 6H).

### PREPARATIVE EXAMPLES 42˜46

The following compounds in Table 2 were prepared using the method described in Preparative Example 41. All compounds gave satisfactory ¹H NMR.

TABLE 2
Preparation of hydroxylamines

| | | |
|---|---|---|
| Pre- | | LC-MS, |
| parative | | min. |
| Example | Compound | (m/Z) |

| | | |
|---|---|---|
| 42 | 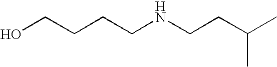 | — |
| 43 | 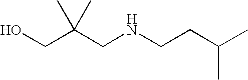 | — |
| 44 | 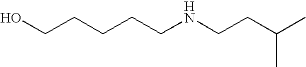 | — |
| 45 | 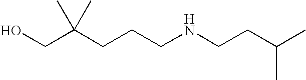 | 0.35(202.1) |
| 46 | 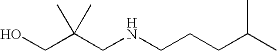 | — |

### PREPARATIVE EXAMPLE 47

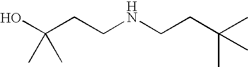

### 4-[(3,3-Dimethylbutyl)amino]-2-methylbutan-2-ol

### Step A. Ethyl N-(3,3-dimethylbutanoyl)-p-alaninate

Add 3.42 g 60% Nay oil dispersion to a solution of ethyl P-alaninate hydrochloride in 150 mL THF at room temperature and stir for 15 minutes. t-Butylacetyl chloride (10 g) was added to the reaction mixture followed by another portion of 3.42 g NaH. The reaction mixture was worked up with aqueous EtOAc after stirring for 6 hours at room temperature to give the title compound, which was used directly in the next step. LC-MS. 1.37 min (m/Z 188.3). ¹H NMR in CD₃OD at 500 MHz: 7.83 (br s, 1H), 3.28 (m, 2H), 2.04 (s, 2H), 1.67 (m, 2H), 1.22 (s, 6H), 1.01 (s, 9H).

### Step B. 4-[(3,3-Dimethylbutyl)amino]-2-methylbutan-2-ol

A solution of 6.0 g ethyl N-(3,3-dimethylbutanoyl)-p-alaninate from the Step A above in 100 mL anhydrous THF was cooled to −78° C. and 38 mL 3 M methylmagnesium bromide in diethyl ether was added over one hour. The reaction mixture was allowed to warm up to room temperature and worked up using aqueous EtOAc to give crude hydroxyl amide intermediate. This crude product was dissolved in 100 mL anhydrous TED and reduced with 4 equiv. of LAH under refluxing for 2 hours. After cooling the reaction mixture, it was quenched with 1:1 water and methanol. The title product was isolated with aqueous EtOAc work-up. LC-MS 0.38 min (m/Z 188.3). ¹H NMR in CDCl₃ at 500 MHz: 2.91 (t, 5.5 Hz, 2H), 2.60 (m, 2H), 1.60 (t, 6.0 Hz, 2H), 1.38 (m, 2H), 1.23 (s, 6H), 0.90 (s, 9H).

### PREPARATIVE EXAMPLES 48˜49

The following compounds in Table 3 were prepared using the method described for Preparative Example 47.

TABLE 3
Preparation of hydroxylamines

| Pre- | | |
|---|---|---|
| parative | | LC-MS, |
| Example | Compound | min. (m/Z) |
| 48 | 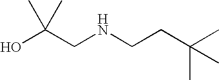 | 0.38 (174.3) |
| 49 | 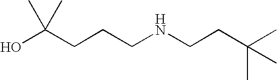 | 0.30 (201.2) |

### PREPARATIVE EXAMPLE 50

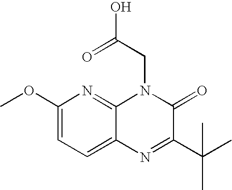

### (2-tert-Butyl-6-methoxy-3-oxopyrido[2,3-b]pyrazin-4(3H)-yl)acetic acid

### Method A:

### Step A. Methyl N-(6-methoxy-3-nitropyridin-2-yl)glycinate

A mixture of 5.0 g 2-chloro-6-methoxy-3-nitropyridine, 4.9 g glycine methyl ester hydrochloride, and 5.0 g DIEA in 200 mL MeOH was stirred at rt for 3 days followed by heating at 50° C. for 1 hour. After cooling, the precipitate was collected by filtration, washed with MeOH, and dried to give the title compound as a yellow solid. Additional crop of the title compound was obtained from the filtrate and wash by evaporation, aqueous work-up, and SGC using 6:1 to 3:1 hexanes and EtOAc. LC-MS: 2.89 min. (m/Z 242). ¹H NMR (CDCl₃, 500 MHz) δ: 8.88 (br s, 1H), 8.35 (d, 8.9 Hz, 1H), 6.15 (d, 9.0 Hz, 1), 4.36 (d, 5.5 Hz, 2H), 3.95 (s, 3H), 3.81 (s, 3H).

### Step B. Methyl N-(3-amino-6-methoxypyridin-2-yl)glycinate

A solution of 2.74 g of methyl N-(6-methoxy-3-nitropyridin-2-yl)glycinate in 100 mL 3:2 mixture of MeOH and THF was treated with 606 mg 10% Pd/C under a hydrogen balloon overnight. The reaction mixture was filtered through a pad of Celite and concentrated to give the title compound. LC-MS: 1.41 min. (m/Z 212). ¹H NMR (CD₃OD, 500 MHz) δ: 6.94 (d, 8.0 Hz, 1H), 5.90 (d, 8.1 Hz, 1H), 4.11 (s, 2H), 3.72 (s, 3H), 3.71 (s, 3H).

### Step C. Methyl (2-tert-butyl-6-methoxy-3-oxopyrido[2,3-b]pyrazin-4(3H)-yl)acetate

A mixture of about 2.1 g crude methyl N-(6-methoxy-3-nitropyridin-2-yl)glycinate and 1.34 g 3,3-dimethyl-2-oxobutanoic acid in 30 mL acetic acid was heated at 48° C. overnight. Some solid side-product (6-methoxy-3,4-dihydropyrido[2,3-b]pyrazin-2(1H)-one) was filtered off and the filtrate was purified on SGC using 7:1 EtOAc and hexanes to give pure title compound. LC-MS: 3.72 min. (m/Z 306.1). ¹H NMR (CDCl₃, 500 MHz) δ: 8.01 (d, 8.7 Hz, 1H), 6.72 (d, 8.7 Hz, 1H), 5.18 (s, 2H), 3.97 (s, 3H), 3.78 (s, 3H), 1.48 (s, 9H). ¹³C NMR (CDCl₃, 125 MHz) δ: 168.73, 163.45, 162.67, 154.69, 141.59, 140.57, 122.68, 107.91, 54.39, 52.82, 42.12, 39.47, 28.02.

The title compound of Step C can also be obtained as follows. A mixture of 50 mg methyl N-(6-methoxy-3-nitropyridin-2-yl)glycinate, 37 mg 3,3-dimethyl-2-oxobutanoic acid, 48 mg HOBt, and 41.7 mg DIEA in 1 mL dry DMF was treated with 79.8 mg EDC. The title compound was isolated on RP-HPLC. Similar result was obtained using PyBOP instead of EDC.

### Step D. (2-tert-Butyl-6-methoxy-3-oxopyrido[2,3-b]pyrazin-4(3H)-yl)acetic acid

A mixture of 680 mg methyl (2-tert-butyl-6-methoxy-3-oxopyrido[2,3-b]pyrazin-4(3H)-yl)acetate in 20 mL MeOH and 2 mL water was treated with 2.2 mL 5 N NaOH solution at room temperature overnight. The mixture was evaporated under vacuum and the residue taken up in 20 mL water and acidified with 6 N HCl to precipitate the title compound. It was obtained as white solid following filtration and drying. LC-MS: 3.25 min. (m/Z 292.1). ¹H NMR (CDCl₃, 500 MHz) δ: 8.02 (d, 8.7 Hz, 1H), 6.74 (d, 8.5 Hz, 2H), 5.22 (s, 2H), 3.98 (s, 3H), 1.47 (s, 9H).

### Method B:

### Step A. tert-Butyl N-(6-methoxy-3-nitropyridin-2-yl)glycinate

A mixture of 3.67 g 2-chloro-6-methoxy-3-nitropyridine, 4.9 g glycine t-butyl ester hydrochloride, and 6.3 g DIEA in 150 mL t-BuOH was refluxed overnight. After cooling, the precipitate (DIEA slat) was removed by filtration. The title compound was obtained from the filtrate and wash by evaporation and aqueous work-up. LC-MS: 3.59 min. (m/Z 228.3). ¹H NMR (CDCl₃, 500 MHz) δ: 8.50 (br s, 1H), 8.35 (d, 9.2 Hz, 1H), 6.13 (d, 9.0 Hz, 1H), 4.27 (d, 5.5 Hz, 2H), 3.98 (s, 3H), 1.51 (s, 9H).

### Step B. tert-Butyl (2-tert-butyl-6-methoxy-3-oxopyrido[2,3-b]pyrazin-4(3H)-yl)acetate

A solution of 0.25 g tert-butyl N-(6-methoxy-3-nitropyridin-2-yl)glycinate in 5 mL THF was treated with a hydrogen balloon with 89 mg 10% Pd/C overnight. LC-MS showed reduction product: 2.34 min. (m/Z 198). The reaction mixture was filtered and the filtrate treated with 172 mg 3,3-dimethyl-2-oxobutanoic acid at room temperature overnight. After evaporation, the residue was purified on SGC using EtOAc-hexanes to give the title compound. LC-MS: 4.19 min. (m/Z 348.1). ¹H NMR (CDCl₃, 500 MHz) δ: 8.00 (d, 8.5 Hz, 1H), 6.70 (d, 8.7 Hz, 1H), 5.07 (s, 2H), 3.99 (s, 3H), 1.48 (s, 9H), 1.46 (s, 9H).

### Step C. (2-tert-Butyl-6-methoxy-3-oxopyrido[2,3-b]pyrazin-4(3H)-yl)acetic acid

tert-Butyl (2-tert-butyl-6-methoxy-3-oxopyrido[2,3-b]pyrazin-4(3H)-yl)acetate (0.10 g) was treated with 1 mL formic acid room temperature overnight. The mixture was evaporated under vacuum to give the title compound identical as that obtained from Method A Step D above.

### PREPARATIVE EXAMPLE 51

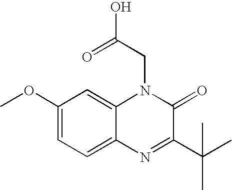

### (3-tert-Butyl-7-methoxy-2-oxoquinoxalin-1(2H)-yl)acetic acid

### Method A.

### Step A. 3-tert-Butyl-7-methoxyquinoxalin-2(1H)-one

The title compound was prepared from 4-methoxybenzene-1,2-diamine and 3,3-dimethyl-2-oxobutanoic acid in HOAc at 48˜49° C. overnight as described in Preparative Example 50 Step C. It was separated from isomeric side-product 3-tert-butyl-6-methoxyquinoxalin-2(1H)-one by SGC. 3-tert-Butyl-7-methoxyquinoxalin-2(1H)-one ¹H NMR (CD₃OD, 500 MHz) δ: 7.67 (d, 8.9 Hz, 1H), 6.90 (dd, 2.7 & 9.0 Hz, 1H), 6.73 (d, 2.8 Hz, 1H), 3.87 (s, 3H), 1.44 (s, 9H).

### Step B. Methyl (3-tert-butyl-7-methoxy-2-oxoquinoxalin-1(2H)-yl)acetate

A solution of 300 mg 3-tert-butyl-7-methoxyquinoxalin-2(1H)-one in 10 mL DMF was treated with 265 mg methyl bromoacetate and 505 mg cesium carbonate at room temperature overnight. It was quenched by adding saturated NH₄Cl solution and worked up by water and EtOAc. The slower-eluting title compound was separated from isomeric faster-eluting methyl [(3-tert-butyl-7-methoxyquinoxalin-2-yl)oxy]acetate using SGC with 7:1 to 3:1 hexanes and EtOAc. Methyl (3-tert-butyl-7-methoxy-2-oxoquinoxalin-1(2H)-yl)acetate ¹H NMR (CD₃OD, 500 MHz) δ: 7.74 (d, 9.0 Hz, 1H), 6.97 (dd, 2.5 & 8.7 Hz, 1H), 6.75 (d, 2.5 Hz, 1H), 5.07 (s, 2H), 3.89 (s, 3H), 3.79 (s, 3H), 1.44 (s, 9H). Its identity was confirmed by NOE spectroscopy using irradiation of the methylene and showing positive NOE at 6.75 ppm.

### Step C. (3-tert-Butyl-7-methoxy-2-oxoquinoxalin-1(2H)-yl)acetic acid

The title compound was prepared from methyl (3-tert-butyl-7-methoxy-2-oxoquinoxalin-1(2H)-yl)acetate using the procedure described in Step D Method A of Preparative Example 50. LC-MS: 3.23 min. (m/Z 291.1). ¹H NMR (CD₃OD, 500 MHz) δ: 7.73 (d, 8.9 Hz, 1H), 6.96 (dd, 2.6 & 9.0 Hz, 1H), 6.74 (d, 2.3 Hz, 1H), 5.02 (s, 2H), 3.90 (s, 3H), 1.45 (s, 9H).

### Method B.

### Step A. tert-Butyl N-(5-methoxy-2-nitrophenyl)glycinate

A mixture of 20.7 g 3-fluoro-4-nitrophenol and 36.2 g potassium carbonate in 130 mL DMF was heated to 50° C. for 15 minutes, cooled to 0° C., and treated with 37.2 g iodomethane This mixture was heated at 60° C. for 2.5 hours at which time LC-MS showed no starting phenol. It was evaporated under reduced pressure to remove any remaining iodomethane. Glycine t-butyl ester hydrochloride (23.8 g) was added to the reaction mixture together with 60 mL DMF and the resulting mixture was allowed to sit at room temperature overnight followed by refluxing for 4 hours. Aqueous work-up with EtOAc afforded the title compound. LC-MS: 3.57 min. (m/Z 283). ¹H NMR (CDCl₃, 500 MHz) δ: 8.66 (br s, 1H), 8.20 (d, 9.4 Hz, 1H), 6.31 (dd, 2.6 & 9.6 Hz, 1H), 6.02 (d, 2.5 Hz, 1H), 3.98 (d, 5.1 Hz, 2H), 3.88 (s, 3H), 1.51 (s, 9H).

### Step B. tert-Butyl (3-tert-butyl-7-methoxy-2-oxoquinoxalin-1(2H)-yl)acetate

A mixture of 3.05 g tert-butyl N-(5-methoxy-2-nitrophenyl)glycinate and 575 mg 10% Pd/C in 125 mL methanol was hydrogenated with hydrogen balloon at room temperature for 3.5 hours. The reaction mixture was filtered to remove the catalyst and the solvent was removed under reduced pressure without heating and replaced with THF. It was treated with 3,3-dimethyl-2-oxobutanoic acid (2.1 g) at room temperature overnight. After removing solvent under reduced pressure, the residue was purified using SGC with 7:1 to 6:1 hexanes and EtOAc go give the title compound. LC-MS: 4.05 min. (m/Z 347.1, 291.0). ¹H NMR (CDCl₃, 500 MHz) δ: 7.77 (d, 8.7 Hz, 1H), 6.90 (dd, 2.3 & 8.7 Hz, 1H), 6.48 (d, 2.5 Hz, 1H), 4.90 (s, 2H), 3.90 (s, 3H), 1.48 (s, 9H), 1.47 (s, 9H).

### Step C. (3-tert-Butyl-7-methoxy-2-oxoquinoxalin-1(2H)-yl)acetic acid

A solution of 1.49 g tert-butyl (3-tert-butyl-7-methoxy-2-oxoquinoxalin-1(2H)-yl)acetate in 50 mL formic acid was allowed to reaction at room temperature for 2 days followed by 2 hours at 50° C. Removing formic acid and drying under vacuum provided the title compound identical to that from Method A above.

### PREPARATIVE EXAMPLE 52

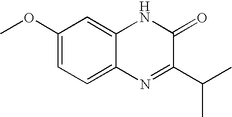

### 3-Isopropyl-7-methoxyquinoxalin-2(1H)-one

The title compound was prepared from 4-methoxybenzene-1,2-diamine and ethyl 3-methyl-2-oxobutanoate in HOAc at room temperature overnight followed by 50° C. for 1.5 hours as described in Preparative Example 51 Method A Step A. It separated from isomeric 3-isopropyl-6-methoxyquinoxalin-2(1H)-one using SGC with 3:1 to 1:1 hexanes and EtOAc. LC-MS: 2.79 min. (m/Z 219). ¹H NMR (CD₃OD, 500 MHz) δ: 7.69 (d, 8.9 Hz, 1H), 6.93 (dd, 2.8 & 9.0 Hz, 11, 6.75 (d, 2.8 Hz, 1H), 3.88 (s, 3H), 3.46˜3.51 (m, 1H), 1.28 (d, 6.9 Hz, 6H).

### PREPARATIVE EXAMPLE 53

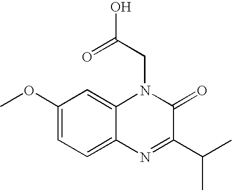

### (3-Isopropyl-7-methoxy-2-oxoquinoxalin-1(2H)-yl)acetic acid

### Step A. tert-Butyl (3-Isopropyl-7-methoxy-2-oxoquinoxalin-1(2H)-yl)acetate

The title compound was prepared from tert-butyl N-(5-methoxy-2-nitrophenyl)glycinate and ethyl 3-methyl-2-oxobutanoate using the method described in Step B Method B of Preparative Example 51. LC-MS: 3.63 min. (m/Z 333.0, 277.0). ¹H NMR (CDCl₃, 500 MHz) δ: 7.78 (d, 8.9 Hz, 1H), 6.92 (dd, 2.5 & 8.7 Hz, 1H), 6.49 (d, 2.5 Hz, 1H), 4.92 (s, 2H), 3.90 (s, 3H), 3.58˜3.61 (m, 1H), 1.48 (s, 6H).

### Step B. (3-Isopropyl-7-methoxy-2-oxoquinoxalin-1(2H)-yl)acetic acid

The title compound was prepared from tert-Butyl (3-Isopropyl-7-methoxy-2-oxoquinoxalin-1(2H)-yl)acetate using the method described in Step C Method B of Preparative Example 51. LC-MS: 2.78 min. (m/Z 277.0). ¹H NMR (CD₃OD, 500 MHz) δ: 7.75 (d, 8.9 Hz, 1H), 6.99 (dd, 2.6 & 9.0 Hz, 1H), 6.78 (d, 2.5 Hz, 1H), 5.07 (s, 2H), 3.90 (s, 3H), 3.54˜3.48 (m, 1H), 1.28 (d, 6.9 Hz, 6H).

### PREPARATIVE EXAMPLE 54

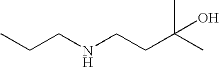

### 2-Methyl-4-(propylamino)butan-2-ol

### Step A. Methyl 3-hydroxy-3-methylbutanoate

A mixture of 5.5 g 3-hydroxy-3-methylbutanoic acid and 18.2 g cesium carbonate in 100 mL DMF was treated with 8.6 g iodomethane at room temperature overnight. The title compound was obtained after aqueous work-up with EtOAc. ¹H NMR (CDCl₃, 500 MHz) δ: 3.74 (s, 3H), 3.51 (s, 1H), 2.52 (s, 2H), 1.30 (s, 6H).

### Step B. 3-Hydroxy-3-methyl-N-propylbutanamide

A solution of 3.94 g methyl 3-hydroxy-3-methylbutanoate and 17.6 g n-propylamine in 50 mL methanol was heated at 75° C. in a sealed tube overnight. After cooling, the tube was opened and the content concentrated under reduced pressure to give crude title compound containing about 30% starting ester. ¹H NMR (CDCl₃, 500 MHz) δ: 3.21-3.25 (m, 2H), 2.32 (s, 2H), 1.52˜1.56 (m, 2H), 1.26 (s, 6H), 0.93 (t, 7.5 Hz, 3H).

### Step C. 2-Methyl-4-(propylamino)butan-2-ol

A solution of 2.3 g crude 3-hydroxy-3-methyl-N-propylbutanamide from Step B in 100 mL toluene was cooled to −30° C. and 7.2 g neat DIBAL was added slowly. After stirring at −10° C. for 10 minutes, the mixture was stirred at room temperature for 2 hours. It was quenched with the addition of 20:1 methanol and water. Add ether to the resulting gel and filter the mixture. Evaporation of the filtrate gave the crude title compound. This crude product was used in excess for amide coupling reactions. LC-MS: 0.47 min. (m/Z 146.1).

### PREPARATIVE EXAMPLE 55

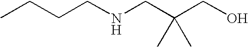

### 3-(Butylamino)-2,2-dimethylpropan-1-ol

### Step A. N-Butyl-3-hydroxy-2,2-dimethylpropanamide

A mixture of 4.54 g 3-hydroxy-2,2-dimethylpropanoic acid, 4.21 g n-butylamine, 7.78 g HOBt, and 14.89 g DIEA in 100 mL DMF was treated with 18.4 g EDC overnight at room temperature. Solvent was removed under reduced pressure and residue was diluted with saturated NH₄Cl solution and extracted with EtOAc. The combined extract was washed with water and saturated brine and concentrated to give the title compound. ¹H NMR (CDCl₃, 500 MHz) δ: 3.57 (s, 2H), 3.24˜3.28 (m, 2H), 1.47˜1.52 (m, 2H), 1.33˜1.38 (m, 2H), 1.19 (s, 6H), 0.94 (t, 7.3 Hz, 3H).

### Step B. 3-(Butylamino)-2,2-dimethylpropan-1-ol

N-Butyl-3-hydroxy-2,2-dimethylpropanamide was reduced with excess LAH in refluxing ether overnight. The reaction mixture was quenched with adding small portions of saturated sodium sulfate solution while cooled with an ice bath. The resulting mixture was filtered and the filtrate concentrated to give the title compound as white solid. LC-MS: 0.7 min. (m/Z 160.1). ¹H NMR (CDCl₃, 500 MHz) δ: 3.50 (s, 2H), 2.62 (s, 2H), 2.59 (t, 7.1 Hz, 2H), 1.45˜1.48 (m, 2H), 1.31˜1.38 (m, 2H), 0.93 (s, 6H), 0.92 (t, 7.3 Hz, 3H).

### PREPARATIVE EXAMPLE 56

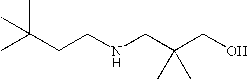

### 3-[(3,3-Dimethylbutyl)amino]-2,2-dimethylpropan-1-ol

The title compound was prepared using the same method described in Preparative Example 54 except the reduction was carried out in refluxing dioxane. LC-MS: 1.69 min. (m/Z 188.3). ¹H NMR (CDCl₃, 500 MHz) δ: 3.52 (s, 2H), 2.64 (s, 2H), 1.45˜1.48 (m, 2H), 1.61 (s, 1H), 2.58˜2.61 (m, 2H), 1.38˜1.42 (m, 2H), 0.95 (s, 6H), 0.92 (s, 9H).

### PREPARATIVE EXAMPLE 57

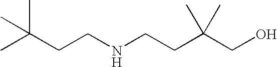

### 4-[(3,3-Dimethylbutyl)amino]-2,2-dimethylbutan-1-ol

The title compound was prepared as described in Preparative Example 55 from 3,3-dimethylbutanoyl chloride and methyl 4-amino-2,2-dimethylbutanoate hydrochloride. LC-MS: 2.09 min. (m/Z 202.3). ¹H NMR (CDCl₃, 500 MHz) δ: 3.25 (s, 2H), 1.58˜1.68 (m, 2H), 2.60˜2.63 (m, 2H), 1.40˜1.45 (m, 4H), 0.92 (s, 6H), 0.91 (s, 9H).

### PREPARATIVE EXAMPLE 58

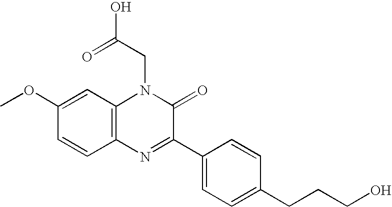

### [3-[4-(3-Hydroxypropyl)phenyl]-7-methoxy-2-oxoquinoxalin-1(2H)-yl]acetic acid

### Step A. 5-Methoxy-N-(4-methoxybenzyl)-2-nitroaniline

The title compound was prepared from 3-fluoro-4-nitrophenol, methyl iodide, and 4-methoxybenzylamine using the procedure described in Preparative Example 51 Method B Step A. LC-MS: 3.44 min. (m/Z 311.0). ¹H NMR (CDCl₃, 500 MHz) δ: 8.63 (s, 1H), 8.18 (d, 9.6 Hz, 1H), 7.29˜7.32 (m, 2H), 6.91˜6.94 (m, 2H), 6.27 (dd, 2.6 & 9.7 Hz, 1H), 6.17 (d, 2.9 Hz, 1H), 4.47 (d, 5.3 Hz, 2H), 3.83 (s, 3H), 3.80 (s, 3H)

### Step B. Methyl {4-[3-(acetyloxy)propyl]phenyl}(oxo)acetate

A solution of 5 g 3-phenyl propylacetate and 4.1 g methyl oxalyl chloride in 20 mL DCM was added slowly to a mixture of 8.2 g anhydrous aluminum chloride in 30 mL DCM at 0° C. The reaction mixture is then allowed to warm up to room temperature overnight. Aqueous work-up with saturated NH₄Cl and EtOAc followed by SGC using 3:1 hexanes and EtOAc afforded the title compound as clear oil. ¹H NMR (CDCl₃, 500 MHz) δ: 7.97 (d, 8.2 Hz, 2H), 7.35 (d, 8.2 Hz, 2H), 4.11 (t, 6.3 Hz, 2H), 3.99 (s, 3H), 2.80 (t, 7.7 Hz, 2H), 2.07 (s, 3H), 1.98˜2.03 (m, 2H).

### Step C. 4-Methoxy-N²-(4-methoxybenzyl)benzene-1,2-diamine

A mixture of 2.5 g 5-methoxy-N-(4-methoxybenzyl)-2-nitroaniline, 7.3 g iron powders, 7.8 g HOAc, 25 mL EtOH, and 70 mL water was refluxed under nitrogen for 1 hour. After cooling, most solvents were removed under reduced pressure. Add 200 mL 2.5 N NaOH to the residue and filter the mixture through a pad of Celite with EtOAc. Separate the layers, wash the organic layer with brine, and evaporate to give the crude title compound, which was used without further purification. LC-MS: 2.39 min. (m/Z 259). ¹H NMR (CDCl₃, 500 MHz) δ: 7.32˜7.35 (m, 2H), 6.90˜6.92 (m, 2H), 6.69 (d, 8.2 Hz, 1H), 6.32 (d, 2.8 Hz, 1H), 6.22 (dd, 2.9 & 8.2 Hz, 1H), 4.25 (s, 2H), 3.83 (s, 3H), 3.75 (s, 3H).

### Step D. 3-{4-[6-Methoxy-4-(4-methoxybenzyl)-3-oxo-3,4-dihydroquinoxalin-2-yl]phenyl}propyl acetate

The title compound was prepared from 4-methoxy-N²-(4-methoxybenzyl)-benzene-1,2-diamine and methyl {4-[3-(acetyloxy)propyl]phenyl}(oxo)acetate in HOAc at 45° C. in 6 hours. Similar reaction in THF was very slow. SGC purification using hexanes-EtOAc afforded pure title compound. LC-MS: 3.97 min. (m/Z 473.1). ¹H NMR (CDCl₃, 500 MHz) δ: 8.29 (d, 8.3 Hz, 2H), 7.87 (d, 8.7 Hz, 1H), 7.29˜7.33 (m, 4H), 6.93 (dd, 2.5 & 8.7 Hz, 1H), 6.87˜6.89 (m, 2H), 6.79 (d, 2.6 Hz, 1H), 5.49 (s, 2H), 4.14 (t, 6.6 Hz, 2H), 3.85 (s, 3H), 3.79 (s, 3H), 2.78 (t, 7.8 Hz, 2H), 2.10 (s, 3H), 1.99˜2.05 (m, 2H).

### Step E. 3-[4-(6-Methoxy-3-oxo-3,4-dihydroquinoxalin-2-yl)phenyl]propyl acetate

Refluxing 0.57 g 3-{4-[6-methoxy-4-(4-methoxybenzyl)-3-oxo-3,4-dihydroquinoxalin-2-yl]phenyl}propyl acetate and 1.4 mL anisole in 10 mL neat TFA for 12 hours or stir it at room temperature in 2% triflic acid in TFA for 3 days gave the title compound. SGC purification gave pure material. LC-MS: 3.32 min. (m/Z 353.1). ¹H NMR (CDCl₃, 500 MHz) δ: 8.32 (d, 8.3 Hz, 2H), 7.85 (d, 8.9 Hz, 1H), 7.33 (d, 8.2 Hz, 2H), 6.98 (dd, 2.3 & 8.9 Hz, 1H), 6.69 (d, 2.6 Hz, 1H), 4.14 (t, 6.6 Hz, 2H), 3.93 (s, 3H), 2.79 (t, 7.7 Hz, 2H), 2.10 (s, 3H), 2.00˜2.05 (m, 2H).

### Step F. 3-[4-(3-Hydroxypropyl)phenyl]-7-methoxyquinoxalin-2(1H)-one

A solution of 297 mg 3-[4-(6-methoxy-3-oxo-3,4-dihydroquinoxalin-2-yl)phenyl]propyl acetate in 10 mL MeOH and 1 mL water was treated with 0.84 mL 5 N NaOH at room temperature overnight. Solvents were removed under reduced pressure, the residue re-dissolved in water, and extracted with ether. The combine organic extract was washed with brine and evaporated to give the title compound. LC-MS: 2.83 min. (m/Z 311.0). ¹H NMR (CD₃OD, 500 MHz) δ: 8.16 (d, 8.3 Hz, 2H), 7.76 (d, 8.9 Hz, 1H), 7.32 (d, 8.2 Hz, 2H), 6.97 (dd, 2.8 & 8.9 Hz 1H), 6.92 (s, 1H), 6.80 (d, 2.5 Hz, 1H), 3.91 (s, 3H), 3.60 (t, 6.6 Hz, 2H), 2.76 (t, 7.7 Hz, 2H), 1.89 (s, 1H), 1.86˜1.91 (m, 2H).

### Step G. Methyl [3-[4-(3-hydroxypropyl)phenyl]-7-methoxy-2-oxoquinoxalin-1(2H)-yl]acetate

The title compound was prepared from 3-[4-(3-hydroxypropyl)phenyl]-7-methoxyquinoxalin-2(1H)-one and methyl bromoacetate using the method described in Preparative Example 51 Method A Step B. It was separated from faster-eluting isomeric methyl ({3-[4-(3-hydroxypropyl)phenyl]-7-methoxyquinoxalin-2-yl}oxy)acetate using SGC with 1:1 to 1:2 hexanes/EtOAc. LC-MS: 3.18 min. (m/Z 383.2). ¹H NMR (CD₃OD, 500 MHz) δ: 8.15 (d, 8.3 Hz, 2H), 7.85 (d, 9.0 Hz, 1H), 7.32 (d, 8.2 Hz, 2H), 7.05 (dd, 2.6 & 9.0 Hz 1H), 6.85 (d, 2.5 Hz, 1H), 5.19 (s, 2H), 4.86 (br s, 1H), 3.93 (s, 3H), 3.81 (s, 3H), 3.60 (t, 6.6 Hz, 2H), 2.76 (t, 7.8 Hz, 2H), 1.89 (s, 1H), 1.86˜1.92 (m, 2H).

### Step H. [3-[4-(3-Hydroxypropyl)phenyl]-7-methoxy-2-oxoquinoxalin-1(2H)-yl]acetic acid

The title compound was prepared from methyl [3-[4-(3-hydroxypropyl)phenyl]-7-methoxy-2-oxoquinoxalin-1(2H)-yl]acetate using procedure described in Step F above. LC-MS: 2.87 min. (m/Z 369.1). ¹H NMR (CD₃OD, 500 MHz) δ: 8.15 (d, 8.2 Hz, 2H), 7.84 (d, 8.9 Hz, 1H), 7.32 (d, 8.2 Hz, 2H), 7.05 (dd, 2.3 & 8.8 Hz, 1H), 6.84 (d, 2.3 Hz, 1H), 5.15 (s, 2H), 3.94 (s, 3H), 3.61 (t, 6.5 Hz, 2H), 2.77 (t, 7.8 Hz, 2H), 1.86˜1.92 (m, 2H). NOE difference spectrum further confirmed its structure.

### PREPARATIVE EXAMPLE 59

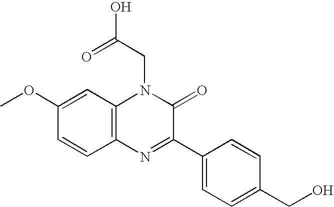

### [3-[4-(hydroxymethyl)phenyl]-7-methoxy-2-oxoquinoxalin-1(2H)-yl]acetic acid

### Step A. Methyl (4-methylphenyl)(oxo)acetate

The title compound was prepared from toluene and methyl oxalyl chloride as described in Preparative Example 57 Step B. ¹H NMR (CDCl₃, 500 MHz) δ: 7.94 (d, 8.2 Hz, 2H), 7.33 (d, 8.0 Hz, 2H), 3.99 (s, 3H), 2.46 (s, 3H).

### Step B. Methyl {4-[(acetyloxy)methyl]phenyl}(oxo)acetate

The title compound was prepared from methyl (4-methylphenyl)(oxo)acetate using the method of Barnish et al. (J. Med. Chem. 1981, 24, 399) in two steps. ¹H NMR (CDCl₃, 500 MHz) δ: 8.05 (d, 8.5 Hz, 2H), 7.51 (d, 8.1 Hz, 2H), 5.2 (s, 2H), 4.01 (s, 3H), 2.16 (s, 3H).

### Step C. 4-[6-Methoxy-4-(4-methoxybenzyl)-3-oxo-3,4-dihydroquinoxalin-2-yl]benzyl acetate

The title compound was prepared from methyl {4-[(acetyloxy)methyl]phenyl}(oxo)acetate and 4-methoxy-N²-(4-methoxybenzyl)benzene-1,2-diamine using method described in Preparative Example 57 Step D. ¹H NMR (CD₃OD, 500 MHz) δ: 8.27 (d, 8.2 Hz, 2H), 7.82 (d, 9.0 Hz, 1H), 7.48 (d, 8.2 Hz, 2H), 7.27 (d, 8.9 Hz, 2H), 6.99 (dd, 2.2 & 8.9 Hz, 1H), 6.89˜6.90 (m, 3H), 5.55 (s, 2H), 5.19 (s, 2H), 3.83 (s, 3H), 3.76 (s, 3H), 2.12 (s, 3H).

### Step D. 3-[4-(Hydroxymethyl)phenyl]-7-methoxyquinoxalin-2(1H)-one

The title compound was prepared from stirring 0.23 g 4-[6-methoxy-4-(4-methoxybenzyl)-3-oxo-3,4-dihydroquinoxalin-2-yl]benzyl acetate overnight in 10 mL TFA containing 0.14 mL triflic acid and 0.56 mL anisole. Evaporation under vacuum, aqueous work-up with EtOAc afforded the title compound and its hydrolyzed alcohol as a mixture. Hydrolysis of this mixture as described in Preparative Example 57 Step F afforded the title compound. LC-MS: 2.40 min. (n/Z 283.2). ¹H NMR (CD₃OD, 500 MHz) δ: 8.03 (d, 8.0 Hz, 2H), 7.61 (d, 9.1 Hz, 1H), 7.43 (d, 8.0 Hz, 2H), 6.88 (d, 2.5 Hz, 1H), 6.81 (dd, 2.5 & 9.0 Hz, 1H), 4.67 (s, 2H), 3.88 (s, 3H).

### Step E. Methyl [3-[4-(hydroxymethyl)phenyl]-7-methoxy-2-oxoquinoxalin-1(2H)-yl]acetate

The title compound was prepared from 3-[4-(hydroxymethyl)phenyl]-7-methoxyquinoxalin-2(11)-one and methyl bromoacetate using the method described in Preparative Example 57 Step G. LC-MS: 2.75 min. (m/Z 355.2). ¹H NMR (CD₃OD, 500 MHz) δ: 8.23 (d, 8.5 Hz, 2H), 7.86 (d, 8.9 Hz, 1H), 7.46 (d, 8.2 Hz, 2H), 7.06 (dd, 2.6 & 9.0 Hz, 1H), 6.86 (d, 2.5 Hz, 1H), 5.20 (s, 2H), 4.69 (s, 2H), 3.94 (s, 3H), 3.81 (s, 3H).

### Step F. [3-[4-(Hydroxymethyl)phenyl]-7-methoxy-2-oxoquinoxalin-1(2H)-yl]acetic acid

The title compound was prepared from methyl [3-[4-(hydroxymethyl)phenyl]-7-methoxy-2-oxoquinoxalin-1(2H)-yl]acetate using the method described in Preparative Example 57 Step H. LC-MS: 2.47 min. (m/Z 341.3). ¹H NMR (CD₃OD, 500 MHz) δ: 8.22 (d, 8.2 Hz, 2H), 7.85 (d, 8.9 Hz, 1H), 7.46 (d, 8.3 Hz, 2H), 7.05 (dd, 2.3 & 8.9 Hz, 1H), 6.84 (d, 2.3 Hz, 1H), 5.16 (s, 2m), 4.69 (s, 31, 3.94 (s, 3H).

### PREPARATIVE EXAMPLE 60

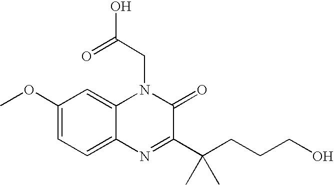

### [3-(4-Hydroxy-1,1-dimethylbutyl)-7-methoxy-2-oxoquinoxalin-1(2R)-yl]acetic acid

### Step A. 3,3-Dimethyl-2-oxohexanedioic acid

The title compound was prepared from 4,4-dimethylcyclohexane-1,3-dione using the method of Langley et al. (J. Chem. Soc. 1962, 2972). It was obtained as a 6:5 mixture of 3,3-dimethyl-2-oxohexanedioic acid and 2,2-dimethylpentanedioic acid based on ¹H NMR and was used in the next step without purification.

### Step B. 4-{4-[6-Methoxy-4-(4-methoxybenzyl)-3-oxo-3,4-dihydroquinoxalin-2-yl]phenyl}-4-methylpentanoic acid

The title compound was prepared from 3,3-dimethyl-2-oxohexanedioic acid and 4-methoxy-N²-(4-methoxybenzyl)benzene-1,2-diamine using method described in Preparative Example 57 Step D. LC-MS: 3.5 min. (m/Z 411.3). ¹H NMR (CDCl₃, 500 MHz) δ: 7.74 (d, 8.9 Hz, 2H), 7.20 (d, 8.4 Hz, 2H), 6.85˜6.87 (m, 3H), 6.70 (d, 2.5 Hz, 1H), 5.39 (s, 2H), 3.81 (s, 3H), 3.78 (s, 3H), 2.33˜2.39 (m, 4H), 1.52 (s, 6H).

### Step C. 4-[4-(6-Methoxy-3-oxo-3,4-dihydroquinoxalin-2-yl)phenyl]-4-methylpentanoic acid

The title compound was prepared from 4-{4-[6-methoxy-4-(4-methoxybenzyl)-3-oxo-3,4-dihydroquinoxalin-2-yl]phenyl}-4-methylpentanoic acid using the method described in the first half of Preparative Example 58 Step D and purified by SGC. LC-MS: 2.75 min. (m/Z 291.2). ¹H NMR (CD₃OD, 500 MHz) δ: 7.68 (d, 8.9 Hz, 1H), 6.90 (dd, 2.8 & 9.0 Hz, 1H), 6.73 (d, 2.5 Hz, 1H), 3.87 (s, 3H), 2.28˜2.31 (m, 2H), 2.14˜2.17 (m, 2H), 1.44 (s, 6H).

### Step D. 3-[4-(4-Hydroxy-1,1-dimethylbutyl)phenyl]-7-methoxyquinoxalin-2(1H)-one

The title compound was prepared from 4-[4-(6-methoxy-3-oxo-3,4-dihydroquinoxalin-2-yl)phenyl]-4-methylpentanoic acid using the method of Soai et al (Synthesis (7), 647, 1987). LC-MS: 2.77 min. (m/Z 277.2). ¹H NMR (CD₃OD, 500 MHz) δ: 7.67 (d, 9.0 Hz, 1H), 6.90 (dd, 2.8 & 8.9 Hz, 1H), 6.73 (d, 2.7 Hz, 1H), 3.87 (s, 3H), 3.48 (t, 6.9 Hz, 2H), 1.98˜2.02 (m, 3H), 1.43 (s, 6H), 1.32˜1.38 (m, 2H).

### Step E. Methyl [3-(4-hydroxy-1,1-dimethylbutyl)-7-methoxy-2-oxoquinoxalin-1(2H)-yl]acetate

The title compound was prepared from 3-[4-(4-hydroxy-1,1-dimethylbutyl)phenyl]-7-methoxyquinoxalin-2(1H)-one and methyl bromoacetate using the method described in Preparative Example 57 Step G. LC-MS: 3.03 min. (m/Z 349.3). ¹H NMR (CD₃OD, 500 MHz)

δ: 7.74 (d, 8.9 Hz, 1H), 6.97 (dd, 2.1 & 8.8 Hz, 1H), 6.76 (d, 2.1 Hz, 1H), 5.07 (s, 2H), 3.89 (s, 3H), 3.78 (s, 3H), 3.46 (t, 6.9 Hz, 2H), 1.94˜2.00 (m, 2H), 1.43 (s, 6H), 1.31˜1.37 (m, 2H).

### Step F. [3-(4-Hydroxy-1,1-dimethylbutyl)-7-methoxy-2-oxoquinoxalin-1(2H)-yl]acetic acid

The title compound was prepared from methyl [3-(4-hydroxy-1,1-dimethylbutyl)-7-methoxy-2-oxoquinoxalin-1(2H)-yl]acetate using the method described in Preparative Example 57 Step H. LC-MS: 2.77 min. (m/Z 335.3). ¹H NMR (CD₃OD, 500 MHz) δ: 7.74 (d, 9.0 Hz, 1H), 6.97 (dd, 2.3 & 8.9 Hz, 1H), 6.75 (d, 2.3 Hz, 1H), 5.03 (s, 2H), 3.90 (s, 3H), 3.46 (t, 6.9 Hz, 2H), 1.98˜2.01 (m, 2H), 1.43 (s, 6H), 1.33˜1.38 (m, 2H).

### PREPARATIVE EXAMPLE 61

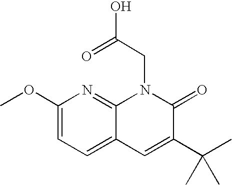

### (3-tert-Butyl-7-methoxy-2-oxo-1,8-naphthyridin-1(2H)-yl)acetic acid

### Step A. Methyl 2-[{2-[(2,2-dimethylpropanoyl)amino]pyridin-3-yl} (hydroxy)methyl]-3,3-dimethylbutanoate

The title compound was prepared from N-(3-formylpyridine-2-yl)-2,2-dimethylpropanamide and methyl 3-methylbutanoate using the method of Turner (J. Org. Chem. 55, 4744, 1990). LC-MS: 2.67 min and 2.76 min. (m/Z 337.3). ¹H NMR of the two diastereomers (CDCl₃, 500 MHz) δ: 8.39-8.40 & 9.36˜8.39 (m, 1H), 8.26 & 8.06 (br s, 1H), 7.91˜7.93 & 7.82˜7.84 (m, 1H), 7.19˜7.24 (m, 1H), 4.09 (d, 5.9 Hz) & 3.75 (br s, 1H), 3.72 &3.26 (s, 3H), 5.01˜5.08 (m, 1H), 2.84˜2.87 (m, 1H), 1.41 & 1.39 (s, 9H), 1.17 & 0.89 (s, 9H).

### Step B. 3-tert-Butyl-1,8-naphthyridin-2(1H)-one

The title compound was prepared from 5.9 g methyl 2-[{2-[(2,2-dimethylpropanoyl)amino]pyridin-3-yl} (hydroxy)methyl]-3,3-dimethylbutanoate in 20 mL dioxane and 35 mL 4 N HCl in dioxane by heating at 170° C. in a microwave reactor for 2 hours in three portions. The solvent was removed from the combined reaction mixture under reduced pressure and the residue was suspended in water and EtOAc after being neutralized to pH 7. The title compound was obtained by filtration. Additional crop of the desired product was obtained from the EtOAc layer following SGC using 2:1 to 1:1 hexanes and EtOAc. LC-MS: 2.69 min. (m/Z 203.2). ¹H NMR (CDCl₃, 500 MHz) δ: 8.46 (dd, 1.6 & 5.6 Hz, 1H), 8.06 (dd, 1.6 & 7.7 Hz, 1H), 7.67 (s, 1H), 7.31 (dd, 5.2 & 7.7 Hz, 1H), 1.46 (s, 9H).

### Step C. 3-tert-Butyl-1,8-naphthyridin-2(1H)-one 8-oxide

The title compound was prepared by heating a solution of 2.05 g 3-tert-butyl-1,8-naphthyridin-2(1H)-one in 20 mL 35% peracetic acid in acetic acid at 50° C. overnight. Aqueous work-up with EtOAc afforded the title compound. LC-MS: 2.20 min. (m/Z 219.1). ¹H NMR (CDCl₃, 500 MHz) δ: 8.53 (d, 6.4 Hz, 1H), 7.79 (d, 7.8 Hz, 1H), 7.65 (s, 1H), 7.25 (dd, 6.7 & 7.8 Hz, 1H), 1.46 (s, 9H).

### Step D. 3-tert-Butyl-7-methoxy-1,8-naphthyridin-2(1H)-one

The title compound was prepared from 3-tert-butyl-1,8-naphthyridin-2(1H)-one 8-oxide using a modified method of Hayashida et al. (Heterocycles, 31 (7), 1325, 1990) with 10 equiv. of pTsCI and 20 equiv. of Et₃N. LC-MS: 3.12 min. (m/Z 233.1). ¹H NMR (CDCl₃, 500 MHz) δ: 7.74 (d, 8.2 Hz, 1H), 7.59 (s, 1H), 6.63 (d, 8.5 Hz, 1H), 4.00 (s, 3H), 1.44 (s, 9H). Its identity was further confirmed by NOE difference spectrum.

### Step E. Methyl (3-tert-butyl-7-methoxy-2-oxo-1,8-naphthyridin-1(2H)-yl)acetate

The title compound was prepared from 3-tert-butyl-7-methoxy-1,8-naphthyridin-2(1H)-one and methyl bromoacetate using the method described in Preparative Example 57 Step G. It was separated from the faster-eluting isomeric methyl [(3-tert-butyl-7-methoxy-1,8-naphthyridin-2-yl)oxy]acetate on RP-HPLC. LC-MS: 3.54 min. (m/Z 305.1). ¹H NMR (CDCl₃, 500 MHz) δ: 7.74 (d, 8.5 Hz, 1H), 7.56 (s, 1H), 6.61 (d, 8.2 Hz, 1H), 5.26 (s, 2H), 3.95 (s, 3H), 3.76 (s, 3H), 1.43 (s, 9H).

### Step F. (3-tert-Butyl-7-methoxy-2-oxo-1,8-naphthyridin-1(2H)-yl)acetic acid

The title compound was prepared from methyl (3-tert-butyl-7-methoxy-2-oxo-1,8-naphthyridin-1(2H)-yl)acetate using the method described in Preparative Example 57 Step H. LC-MS: 3.12 min. (m/Z 291.1). ¹H NMR (CDCl₃, 500 MHz) δ: 7.91 (d, 8.4 Hz, 1H), 7.72 (s, 1H), 6.65 (d, 8.5 Hz, 1H), 5.16 (s, 2H), 3.95 (s, 3H), 1.39 (s, 9H).

### EXAMPLE 1

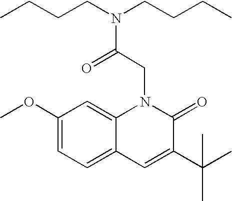

### N,N-Dibutyl-2-(3-tert-butyl-7-methoxy-2-oxoquinolin-1(2H)-yl)acetamide

The title compound was prepared from 6 mg (3-tert-butyl-7-methoxy-2-oxoquinolin-1(2H)-yl)acetic acid, 6 μL di-n-butylamine, 8 mg EDC, 6 mg HOBt, and 15 μL DIEA in 1 mL DMF at room temperature and purified using preparative HPLC followed by lyophilization. LC-MS: 4.27 min. (m/Z 423.3).

### EXAMPLES 2˜47

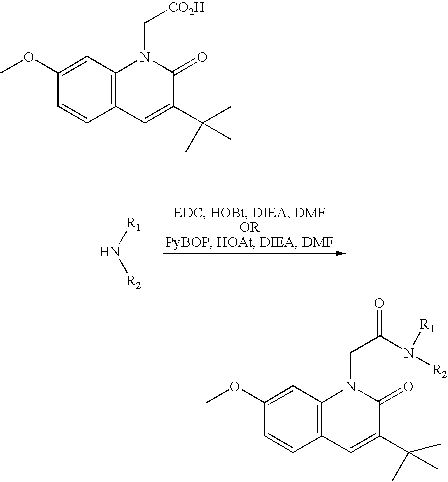

Utilizing the method described in Example 1 using (3-tert-butyl-7-methoxy-2-oxoquinolin-1(2H)-yl)acetic acid and the amine listed in Table 4 below Examples 2 through 47 in Table 4 were prepared. In Examples 32-36, PyBOP and HOAt were used instead of EDC and HOBt.

TABLE 4

| | | LC-MS, |
|---|---|---|
| | | min. |
| Example | Staring amine (HNR₁R₂) | (m/Z) |
| 2 | 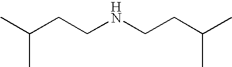 | 4.50(451.3) |
| 3 | 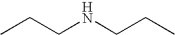 | 3.94(395.3) |
| 4 | 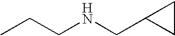 | 3.97(407.2) |
| 5 | 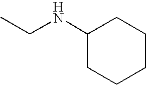 | 4.11(421.3) |
| 6 | 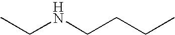 | 3.95(395.2) |
| 7 | 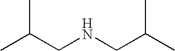 | 4.18(423.3) |
| 8 | 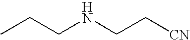 | 3.59(406.3) |
| 9 | 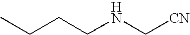 | 3.81(406.2) |
| 10 | 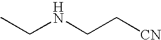 | 3.42(392.2) |
| 11 | 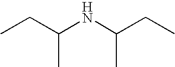 | 4.20(423.3) |
| 12 | 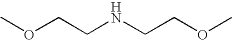 | 3.52(427.2) |
| 13 | 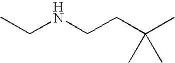 | 4.18(423.3) |
| 14 | 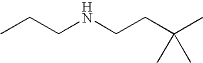 | 4.31(437.3) |
| 15 | 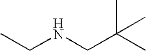 | 4.05(409.3) |
| 16 | 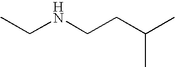 | 4.03(409.3) |
| 17 | 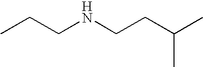 | 4.15(423.2) |
| 18 | 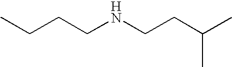 | 4.24(437.2) |
| 19 | 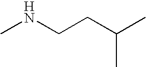 | 3.84(395.2) |
| 20 | 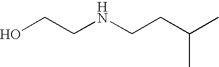 | 3.59(425.2) |
| 21 | 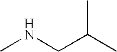 | 3.67(381.3) |
| 22 | 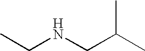 | 3.83(395.3) |
| 23 | 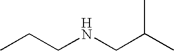 | 3.99(409.2) |
| 24 | 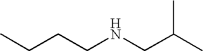 | 4.17(423.3) |
| 25 | 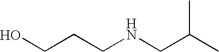 | 3.40(425.3) |
| 26 | 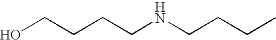 | 3.50(439.4) |
| 27 | 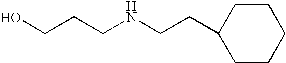 | 3.79(465.3) |
| 28 | 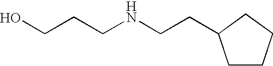 | 3.88(465.5) |
| 29 | 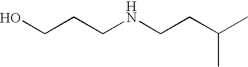 | 3.64(439.3) |
| 30 | 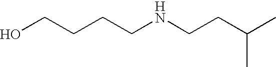 | 3.63(453.2) |
| 31 | 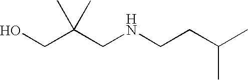 | 4.06(467.4) |
| 32 | 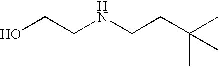 | 3.67(439.2) |
| 33 | 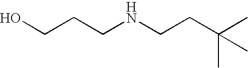 | 3.74(453.3) |
| 34 | 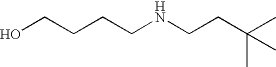 | 3.75(467.3) |
| 35 | 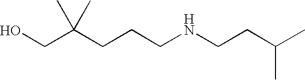 | 4.02(495.3) |
| 36 | 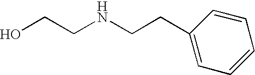 | 3.53(459.2) |
| 37 | 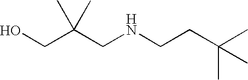 | 3.53(459.5) |
| 38 | 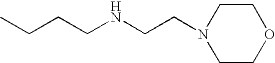 | 4.15(458.4) |
| 39 | 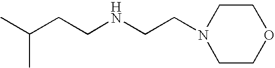 | 3.31(472.4) |
| 40 | 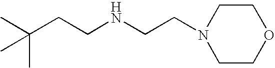 | 3.46(486.5) |
| 41 | 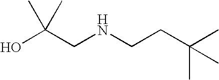 | 3.78(467.3) |
| 42 | 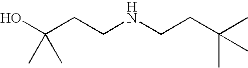 | 3.87(481.3) |
| 43 | 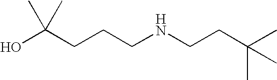 | 3.92(495.4) |
| 44 | 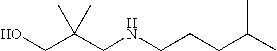 | 4.14(481.4) |
| 45 | 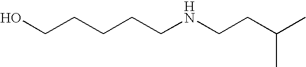 | 3.96(495.4) |
| 46 | 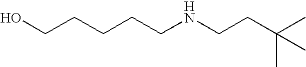 | 3.84(481.4) |
| 47 | 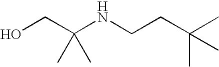 | 3.38(445.3) |

### EXAMPLE 48

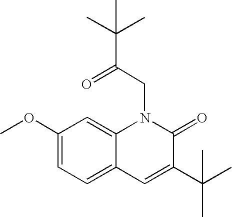

### 3-Tert-butyl-1-(3,3-dimethyl-2-oxobutyl)-7-methoxyquinolin-2(1H)-one

The title compound was prepared from 60 mg 3-tert-butylquinolin-2(1H)-one (Preparative Example 1, Step E), 70 μL 1-bromo-3,3-dimethylbutan-2-one, and 254 mg cesium carbonate in 2 mL DMF at room temperature for 3 hours. It was separated from side-product 1-[(3-tert-butyl-7-methoxyquinolin-2-yl)oxy]-3,3-dimethylbutan-2-one using RP-HPLC. The isomers were identified by comparison of NMR with isomers in Preparative Example 1, Step F. ¹H NMR in CD₃OD at 500 MHz: 1.75 (s, 1H), 7.58 (d, 7.5 Hz, 1H), 6.86 (dd, 7.5 & 2.0 Hz, 1H), 6.47 (d, 2.0 Hz, 1H), 5.44 (s, 2H), 3.84 (s, 3M), 1.40 (s, 9H), 1.37 (s, 9H).

### EXAMPLE 49

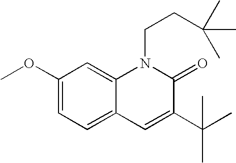

### 3-Tert-butyl-1-(3,3-dimethylbutyl)-7-methoxyquinolin-2(1H)-one

The title compound was prepared from 50 mg 3-tert-butylquinolin-2(1H)-one (Preparative Example 1, Step E), 55 mg 1-bromo-3,3-dimethylbutane, and 143 mg cesium carbonate in 1 mL DMF at 55° C. for 12 hours. It was separated from less polar side-product 3-tert-butyl-2-(3,3-dimethylbutoxy)-7-methoxyquinoline using RP-HPLC. The isomers were identified by comparison of NMR with isomers in Preparative Example 1, Step F. ¹H NMR in CDCl₃ at 500 MHz: 7.53 (s, 1H), 7.46 (d, 9.0 Hz, 1H), 6.81 (dd, 9.0 & 2.0 Hz, 1H), 6.77 (d, 2.0 Hz, 1H), 4.29 (m, 2H), 3.92 (s, 3H), 1.68 (m, 2H), 1.44 (s, 9H), 1.26 (s, 9H).

### EXAMPLE 50

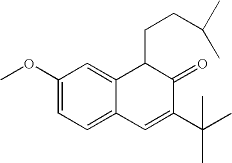

### 3-Tert-butyl-7-methoxy-1-(3-methylbutyl)quinolin-2(1H)-one

The title compound was prepared from 50 mg 3-tert-butylquinolin-2(1H)-one (Preparative Example 1, Step E), 50 mg 1-bromo-3,3-dimethylbutane, and 143 mg cesium carbonate in 1 mL DMF at 55° C. for 12 hours. It was separated purified from less polar side-product 3-tert-butyl-2-(3-methylbutoxy)-7-methoxyquinoline using HPLC. ¹H NMR in CDCl₃ at 500 MHz: 7.56 (s, 1H), 7.48 (d, 8.5 Hz, 1H), 6.83 (dd, 8.5 & 2.0 Hz, 1H), 6.78 (d, 2.0 Hz, 1H), 4.28 (m, 2H), 3.93 (s, 3H), 1.82 (m, 1H), 1.66 (m, 2H), 1.44 (s, 9H), 1.07 (d, 6.5 Hz, 6H).

### EXAMPLE 50

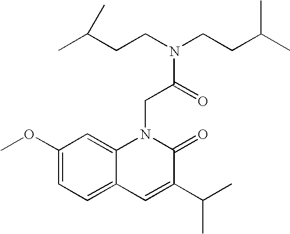

### 2-(3-Isopropyl-7-methoxy-2-oxoquinolin-1(2H)-yl)-N,N-bis(3-methylbutyl)acetamide

The title compound was prepared from (3-isopropyl-2-oxoquinolin-1(2H)-yl)acetic acid from Preparative Example 2, EDC, HOBt, and DIEA using the method in Example 1, purified on HPLC, and re-crystallized from EtOAc-hexanes to give colorless crystals. LC-MS: 4.17 min. (m/Z 437.1). ¹H NMR in CDCl₃ at 500 MHz: 7.49 (s, 1H), 7.46 (d, 8.5 Hz, 1H), 6.81 (dd, 8.5 & 2.0 Hz, 1H), 6.60 (d, 2.0 Hz, 1HU), 5.15 (s, 2H), 3.88 (s, 3H), 3.43 (m, 2H), 3.38 (m, 2H), 3.28 (m, 1H), 1.66˜1.47 (m, 5H), 1.26 (d, 7.0 Hz, 6M), 1.00 (d, 6.5 Hz, 6H), 0.92 (d, 6.5 Hz, 6H).

### EXAMPLES 51˜97

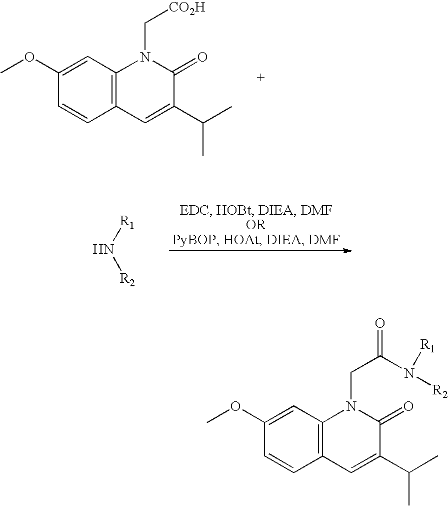

The following compounds in Table 5 were prepared using the method described in Example 1 using (3-isopropyl-2-oxoquinolin-1(2H)-yl)acetic acid and the amine listed in Table 5. For Examples 76 and 92˜97, PyBOP and HOAt were used instead of EDC and HOBt.

TABLE 5

| | | LC-MS, |
|---|---|---|
| | | min. |
| Example | Starting amine (HNR₁R₂) | (m/Z) |
| 51 | 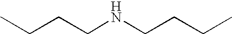 | 3.95(409.1) |
| 52 | 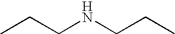 | 3.58(381.1) |
| 53 | 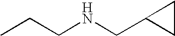 | 3.62(393.1) |
| 54 | 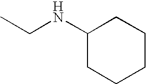 | 3.79(407.1) |
| 55 | 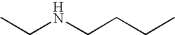 | 3.59(381.1) |
| 56 | 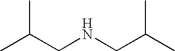 | 3.90(409.2) |
| 57 | 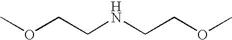 | 3.20(413.1) |
| 58 | 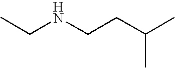 | 3.75(395.2) |
| 59 | 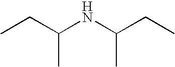 | 3.91(409.1) |
| 60 | 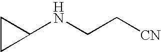 | 3.23(390.1) |
| 61 | 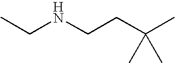 | 3.91(409.2) |
| 62 | 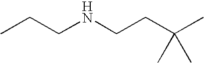 | 4.05(423.2) |
| 63 | 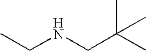 | 3.71(395.2) |
| 64 | 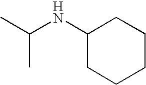 | 4.00(421.2) |
| 65 | 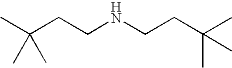 | 4.35(465.2) |
| 66 | 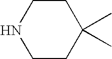 | 3.53(393.1) |
| 67 | 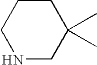 | 3.58(393.1) |
| 68 | 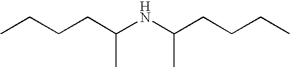 | 4.43(465.2) |
| 69 | 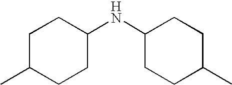 | 4.52(489.1) |
| 70 | 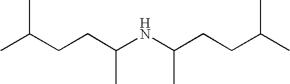 | 4.66(493.1) |
| 71 | 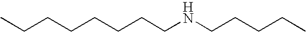 | 4.64(479.2) |
| 72 | 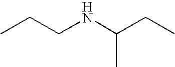 | 3.72(395.1) |
| 73 | 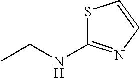 | 3.49(408.0) |
| 74 | 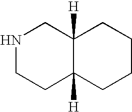 | 3.76(419.1) |
| 75 | 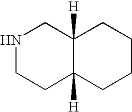 | 3.82(419.1) |
| 76 | 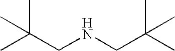 | 4.07(437.1) |
| 77 | 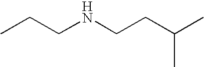 | 3.89(409.2) |
| 78 | 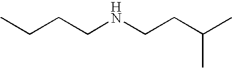 | 4.05(423.2) |
| 79 | 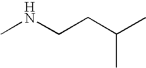 | 3.54(381.3) |
| 80 | 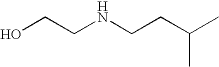 | 3.33(411.4) |
| 81 | 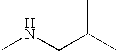 | 3.39(367.3) |
| 82 | 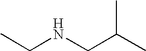 | 3.58(381.3) |
| 83 | 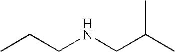 | 3.73(395.3) |
| 84 | 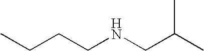 | 3.93(409.3) |
| 85 | 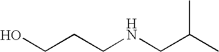 | 3.17(411.3) |
| 86 | 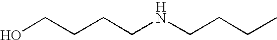 | 3.24(425.4) |
| 87 | 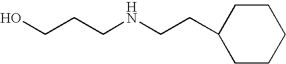 | 3.76(465.3) |
| 88 | 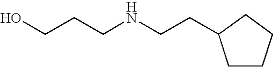 | 3.63(451.2) |
| 89 | 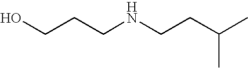 | 3.37(425.3) |
| 90 | 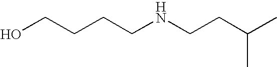 | 3.41(439.2) |
| 91 | 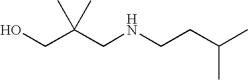 | 3.81(453.4) |
| 92 | 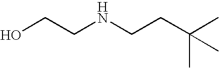 | 3.41(425.2) |
| 93 | 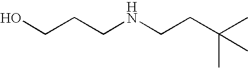 | 3.49(439.2) |
| 94 | 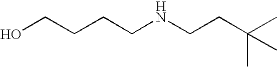 | 3.54(453.3) |
| 95 | 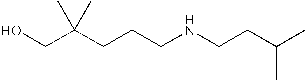 | 3.79(481.3) |
| 96 | 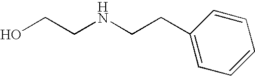 | 3.33(445.3) |
| 97 | 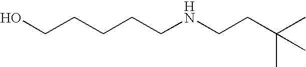 | 3.61(467.4) |

### EXAMPLE 98

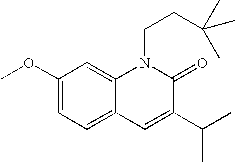

### 1-(3,3-Dimethylbutyl)-3-isopropyl-7-methoxyquinolin-2(1H)-one

The title compound was prepared using the method described in Example 49. LC-MS: 4.16 min. (m/Z 302.2). ¹H NMR in CDCl₃ at 500 MHz: 7.47 (d, 8.5 Hz, 1H), 7.45 (s, 1H), 6.82 (dd, 8.5 & 2.0 Hz, 1H), 6.80 (d, 2.0 Hz, 1H), 4.33 (m, 2H), 3.92 (s, 3H), 3.30 (m, 2H), 1.66 (m, 2H), 1.26 (d, 7.0 Hz, 6H), 1.12 (s, 9H).

### EXAMPLE 99

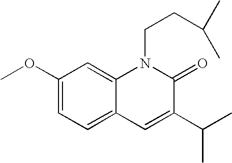

### 3-Isopropyl-7-methoxy-1-(3-methylbutyl)quinolin-2(1H)-one

The title compound was prepared using the method described in Example 50. LC-MS: 4.05 min. (m/Z 288.2). ¹H NMR in CDCl₃ at 500 MHz: 7.48 (d, 8.5 Hz, 1H), 7.47 (s, 1H), 6.84 (dd, 8.5 & 2.0 Hz, 1H), 6.81 (d, 2.0 Hz, 1H), 4.32 (m, 2H), 3.93 (s, 3H), 3.30 (m, 1H), 1.83 (m, 1H), 1.66 (m, 2H), 1.26 (d, 7.0 Hz, 6H), 1.07 (d, 6H).

### EXAMPLE 100

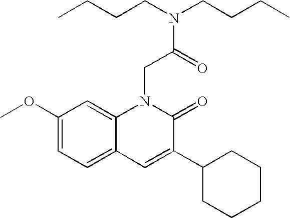

### N,N-Dibutyl-2-(3-cyclohexyl-7-methoxy-2-oxoquinolin-1(2H)-yl)acetamide

The title compound was prepared from (3-cyclohexyl-2-oxoquinolin-1(2H)-yl)acetic acid from Preparative Example 3, EDC, HOBt, and DIEA using the method in Example 1, purified on HPLC, and re-crystallized from EtOAc-hexanes to give colorless crystals. ¹H NMR in CDCl₃ at 500 MHz: 7.47 (s, 1H), 7.45 (d, 8.5 Hz, 1H), 6.80 (dd, 8.5 & 2.0 Hz, 1H), 6.63 (d, 2.0 Hz, 1H), 5.15 (s, 2H), 3.87 (s, 3H), 3.42 (t, 7.0 Hz, 2H), 3.68 (t, 7.0 Hz, 2H), 2.94 (m, 1H), 1.98 (d, 12 Hz, 2H), 1.84 (m, 2H), 1.64 (m, 2H), 1.56 (m, 2H), 1.48 (m, 2H), 1.40 (m, 2H), 1.35-1.25 (m, 6H), 0.99 (t, 7.5 Hz, 3H), 0.91 (t, 7.5 Hz, 3H).

### EXAMPLES 101-124

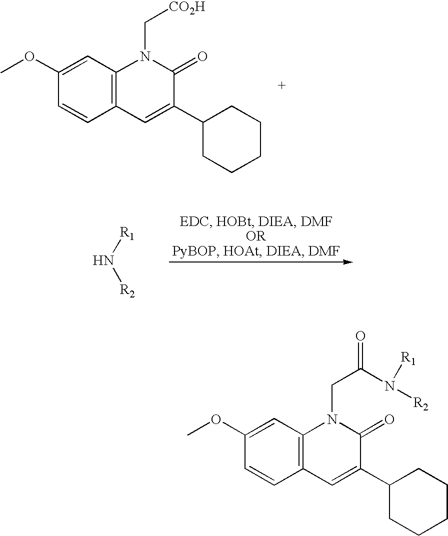

The following compounds in Table 6 were prepared using the method described in Example 1 using (3-cyclohexyl-2-oxoquinolin-1(2H)-yl)acetic acid and the amine listed in Table 6. For Example 124, PyBOP and HOAt were used instead of EDC and HOBt.

TABLE 6

| | | LC-MS, |
|---|---|---|
| Example | Starting amine (HNR₁R₂) | min. (m/z) |
| 101 | 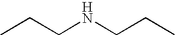 | 4.04 (421.2) |
| 102 | 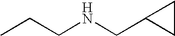 | 4.03 (433.3) |
| 103 | 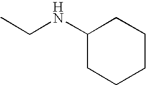 | 4.20 (447.2) |
| 104 | 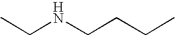 | 4.04 (421.2) |
| 105 | 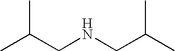 | 4.25 (449.2) |
| 106 | 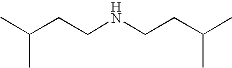 | 4.54 (477.2) |
| 107 | 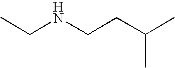 | 4.18 (435.2) |
| 108 | 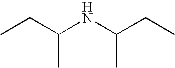 | 4.30 (449.3) |
| 109 | 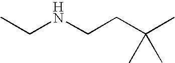 | 4.28 (449.3) |
| 110 | 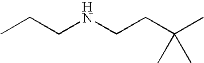 | 4.41 (463.3) |
| 111 | 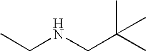 | 4.14 (435.3) |
| 112 | 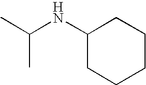 | 4.38 (461.3) |
| 113 | 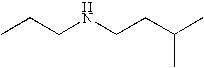 | 4.23 (449.2) |
| 114 | 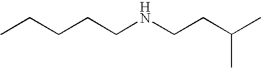 | 4.34 (463.2) |
| 115 | 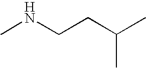 | 4.36 (463.3) |
| 116 | 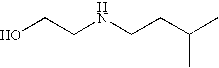 | 3.70 (451.2) |
| 117 | 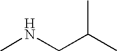 | 3.81 (407.4) |
| 118 | 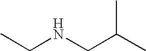 | 3.95 (421.3) |
| 119 | 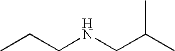 | 4.10 (435.3) |
| 120 | 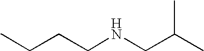 | 4.22 (449.3) |
| 121 | 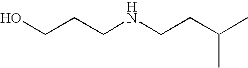 | 3.80 (465.3) |
| 122 | 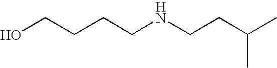 | 3.80 (479.3) |
| 123 | 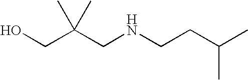 | 4.14 (493.4) |
| 124 | 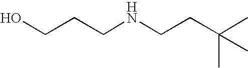 | 3.90 (479.3) |

### EXAMPLE 125

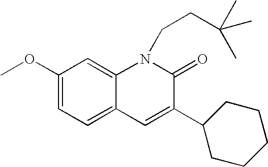

### 3-Cyclohexyl-1-(3,3-dimethylbutyl)-7-methoxyquinolin-2(1H)-one

The title compound was prepared using the method described in Example 49. LC-MS: 4.61 min. (m/Z 342.2). ¹H NMR in CDCl₃ at 500 MHz: 7.48 (d, 8.5 Hz, 1H), 7.46 (s, 1H), 6.85 (dd, 8.5 Hz, 2.0 Hz, 1H), 6.82 (d, 2.0 Hz, 1H), 4.34 (m, 2H), 3.93 (s, 3H), 2.95 (m, 1H), 1.96 (d, 12 Hz, 2H), 1.85 (m, 2H), 1.66 (m, 2H), 1.49 (m, 2H), 1.29 (m, 4H), 1.17 (s, 9H).

### EXAMPLE 126

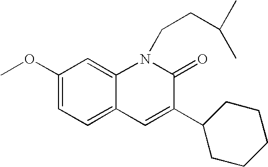

### 3-Cyclohexyl-7-methoxy-1-(3-methylbutyl)quinolin-2(1H)-one

The title compound was prepared using the method described in Example 50. LC-MS: 4.47 min. (m/Z 328.3). ¹H NMR in CDCl₃ at 500 MHz: 7.48 (d, 8.5 Hz, 1H), 7.46 (s, 1H), 6.85 (dd, 8.5 & 2.0 Hz, 1H), 6.82 (d, 2.0 Hz, 1H), 4.32 (m, 2H), 3.93 (s, 3H), 2.95 (m, 1H), 1.96 (d, 12 Hz, 2H), 1.85 (m, 2H), 1.66 (m, 2H), 1.49 (m, 2H), 1.29 (m, 4H), 1.06 (d, 7.5 Hz, 6H).

### EXAMPLE 127

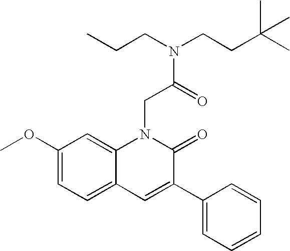

### N-(3,3-Dimethylbutyl)-2-(7-methoxy-2-oxo-3-phenylquinolin-1(2H)-yl)-N-propylacetamide

The title compound was prepared from (2-oxo-3-phenylquinolin-1(2H)-yl)acetic acid from Preparative Example 4, EDC, HOBt, and DIEA using the method in Example 1, purified on HPLC, and re-crystallized from EtOAc-hexanes to give colorless crystals. ¹H NMR in CDCl₃ at 500 MHz: 7.80 (d, 1.5 Hz, 1H), 7.72 (m, 2H), 7.54 (dd, 8.5 Hz, 1.5 Hz, 1H), 7.43 (m, 2H), 7.35 (m, 1H), 6.85 (dd, 8.5 & 1.5 Hz, 1H), 6.63 (m 1H), 5.19 (s, 2H), 3.90 (s, 3H), 3.40 (m, 4H), 1.77-1.48 (m, 4H), 1.03 (t, 7.5 Hz, 3H), 0.92 (s, 9H).

### EXAMPLES 128-140

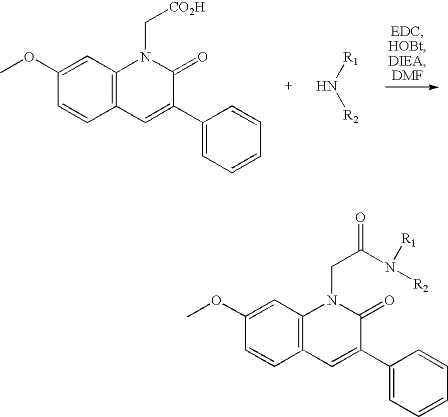

The following compounds in Table 7 were prepared using the method described in Example 1 using (2-oxo-3-phenylquinolin-1(2H)-yl)acetic acid and the amine listed in Table 7.

TABLE 7

| Example | Starting amine (HNR₁R₂) | LC-MS, min. (m/Z) |
|---|---|---|
| 128 | 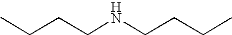 | 4.03 (443.1) |
| 129 | 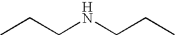 | 3.71 (415.1) |
| 130 | 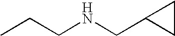 | 3.73 (427.1) |
| 131 | 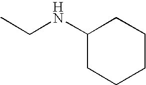 | 3.88 (441.1) |
| 132 | 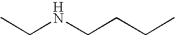 | 3.72 (415.2) |
| 133 | 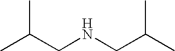 | 3.98 (443.1) |
| 134 | 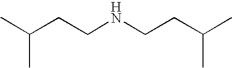 | 4.27 (471.2) |
| 135 | 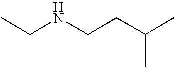 | 3.86 (429.2) |
| 136 | 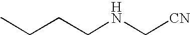 | 3.59 (426.1) |
| 137 | 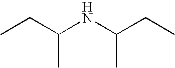 | 3.99 (443.2) |
| 138 | 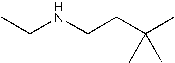 | 3.96 (443.2) |
| 139 | 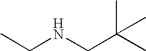 | 3.77 (429.1) |
| 140 | 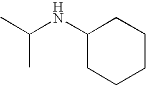 | 4.06 (455.2) |

### EXAMPLE 141

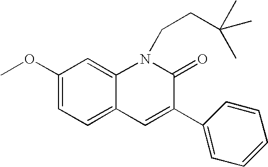

### 1-(3,3-Dimethylbutyl)-7-methoxy-3-phenylquinolin-2(1H)-one

The title compound was prepared using the method described in Example 49. LC-MS: 4.26 min. (m/Z 336.2). ¹H NMR in CDCl₃ at 500 MHz: 7.76 (s, 1H), 7.72 (d, 7.5 Hz, 2H), 7.55 (d, 8.5 Hz, 1H), 7.44 (m, 2H), 7.37 (m, 1H), 6.85 (m, 2H), 4.38 (m, 2H), 3.95 (s, 3H), 1.71 (m, 2H), 1.13 (s, 9H).

### EXAMPLE 142

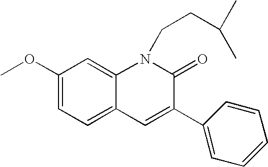

### 7-Methoxy-1-(3-methylbutyl)-3-phenylquinolin-2(1H)-one

The title compound was prepared using the method described in Example 50. LC-MS: 4.10 min. (m/Z 322.2). ¹H NMR in CDCl₃ at 500 MHz: 7.80 (s, 1H), 7.68 (m, 2H), 7.58 (d, 8.5 Hz, 1H), 7.44 (m, 2H), 7.38 (m, 1H), 6.91 (dd, 8.5 & 2.0 Hz, 1H), 6.88 (d, 2.0 Hz, 1H), 4.39 (m, 2H), 3.97 (s, 3H), 1.85 (m, 1H), 1.71 (m, 2H), 1.08 (d, 7.5 Hz, 6H).

### EXAMPLES 143-173

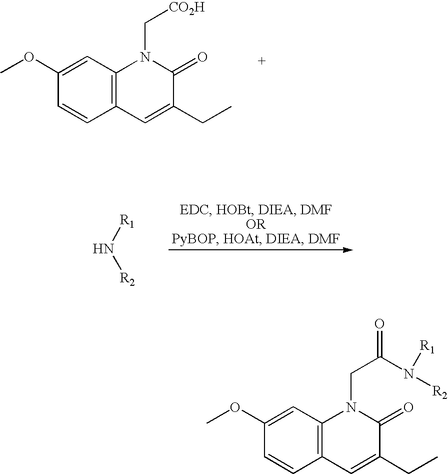

The following compounds in Table 8 were prepared using the method described in Example 1 using (3-ethyl-7-methoxy-2-oxoquinolin-1(2H)-yl)acetic acid from Preparative Example 8 and the amine listed in Table 8. For Examples 172˜173, PyBOP and HOAt were used instead of EDC and HOBt.

TABLE 8

| Ex- | | |
|---|---|---|
| am- | | LC-MS, |
| ple | Starting amine (HNR₁R₂) | min. (m/Z) |
| 143 | 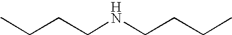 | 3.73 (395.1) |
| 144 | 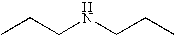 | 3.36 (367.0) |
| 145 | 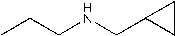 | 3.41 (379.1) |
| 146 | 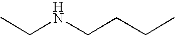 | 3.67 (367.1) |
| 147 | 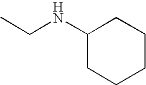 | 3.56 (393.0) |
| 148 | 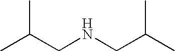 | 3.67 (395.1) |
| 149 | 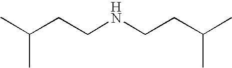 | 4.01 (423.1) |
| 150 | 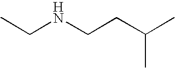 | 3.55 (381.0) |
| 151 | 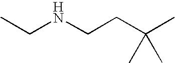 | 3.68 (395.1) |
| 152 | 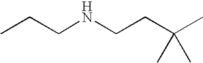 | 3.84 (409.1) |
| 153 | 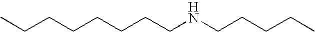 | 4.49 (465.2) |
| 154 | 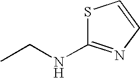 | 3.29 (394.0) |
| 155 | 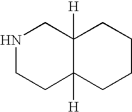 | 3.58 (405.1) |
| 156 | 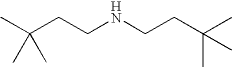 | 4.18 (451.1) |
| 157 | 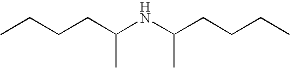 | 4.31 (451.1) |
| 158 | 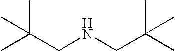 | 3.90 (423.1) |
| 159 | 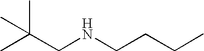 | 381 (409.1) |
| 160 | 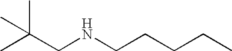 | 4.00 (423.1) |
| 161 | 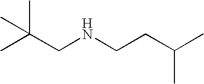 | 3.95 (423.1) |
| 162 | 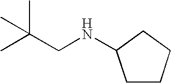 | 3.88 (421.1) |
| 163 | 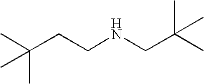 | 4.05 (437.1) |
| 164 | 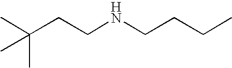 | 3.94 (423.1) |
| 165 | 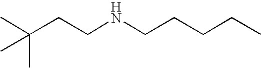 | 4.09 (437.1) |
| 166 | 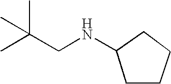 | 3.98 (435.1) |
| 167 | 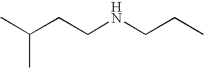 | 3.72 (395.2) |
| 168 | 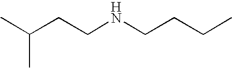 | 3.87 (409.1) |
| 169 | 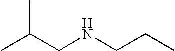 | 3.54 (381.3) |
| 170 | 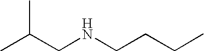 | 3.73 (395.3) |
| 171 | 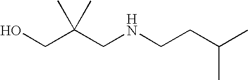 | 3.03 (417.3) |
| 172 | 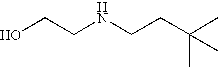 | 3.24 (411.2) |
| 173 | 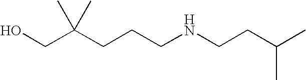 | 3.61 (467.1) |

### EXAMPLES 174-184

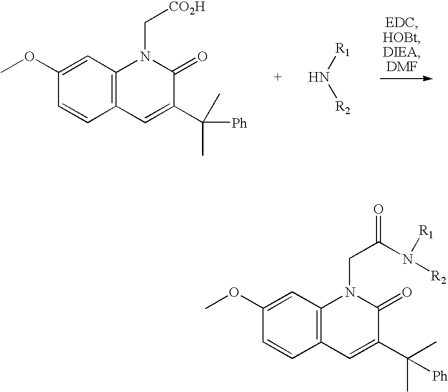

The following compounds in Table 9 were prepared using the method described in Example 1 using [7-methoxy-3-(1-methyl-1-phenylethyl)-2-oxoquinolin-1(2H)-yl]acetic acid from Preparative Example 9 and the amines listed in Table 9.

TABLE 9

| Example | Starting amine (HNR₁R₂) | LC-MS, min. (m/Z) |
|---|---|---|
| 174 | 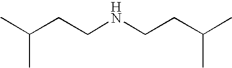 | 4.35 (513.1) |
| 175 | 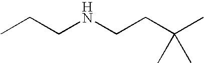 | 4.23 (499.1) |
| 176 | 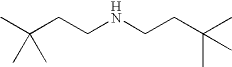 | 4.49 (541.1) |
| 177 | 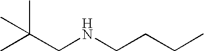 | 4.35 (499.3) |
| 178 | 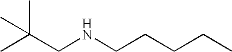 | 4.50 (513.3) |
| 179 | 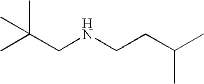 | 4.47 (513.3) |
| 180 | 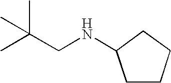 | 4.43 (525.2) |
| 181 | 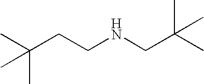 | 4.51 (527.5) |
| 182 | 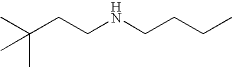 | 4.43 (513.5) |
| 183 | 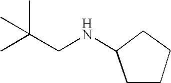 | 4.29 (511.2) |
| 184 | 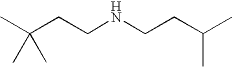 | 4.44 (527.1) |

### EXAMPLES 185˜192

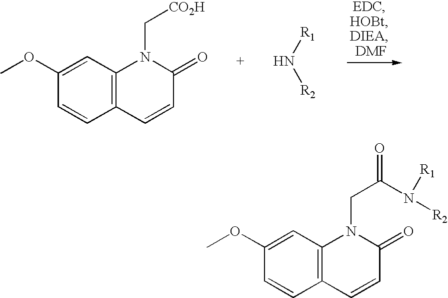

The following compounds in Table 10 were prepared using the method described in Example 1 using (7-methoxy-2-oxoquinolin-1(2H)-yl)acetic acid from Preparative Example 10 and the amine listed in Table 10.

TABLE 10

| Example | Starting amine (HNR₁R₂) | LC-MS, min. (m/Z) |
|---|---|---|
| 185 | 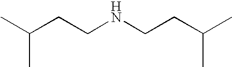 | 3.59 (395.1) |
| 186 | 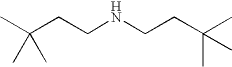 | 3.80 (423.1) |
| 187 | 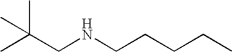 | 3.58 (395.2) |
| 188 | 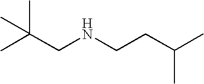 | 3.56 (395.2) |
| 189 | 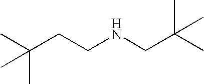 | 3.68 (409.3) |
| 190 | 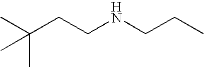 | 3.40 (381.1) |
| 191 | 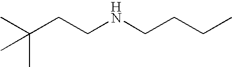 | 3.57 (395.2) |
| 192 | 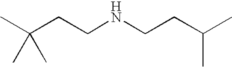 | 3.71 (409.2) |

### EXAMPLES 193˜199

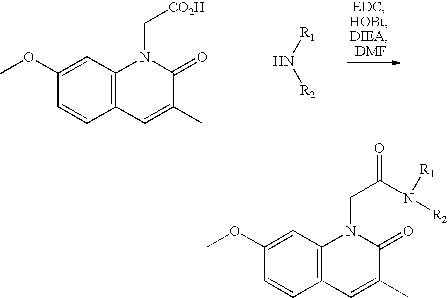

The following compounds in Table 11 were prepared using the method described in Example 1 using (7-methoxy-3-methyl-2-oxoquinolin-1(2H)-yl)acetic acid from Preparative Example 36 and the amine listed in Table 11.

TABLE 11

| | | LC-MS, |
|---|---|---|
| Example | Starting amine (HNR₁R₂) | min. (m/Z) |
| 193 | 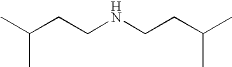 | 3.85 (409.1) |
| 194 | 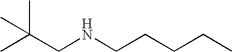 | 3.66 (395.1) |
| 195 | 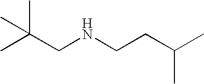 | 3.81 (409.1) |
| 196 | 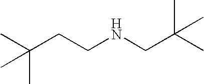 | 3.93 (423.1) |
| 197 | 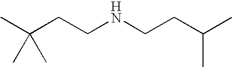 | 3.96 (423.1) |
| 198 | 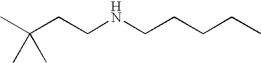 | 3.99 (423.1) |
| 199 | 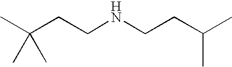 | 3.96 (423.1) |

### EXAMPLES 200˜240

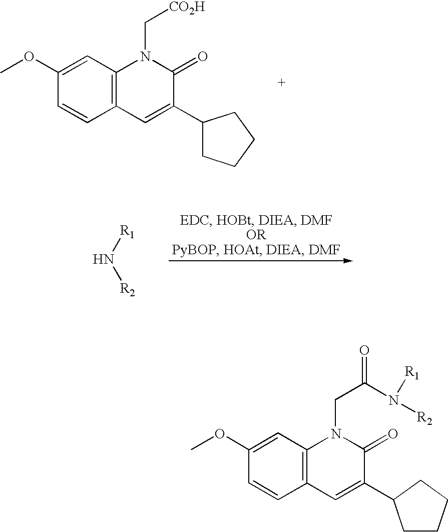

The following compounds in Table 12 were prepared using the method described in Example 1 using (3-cyclopentyl-7-methoxy-2-oxoquinolin-1(2H)-yl)acetic acid from Preparative Example 35 and the amine listed in Table 12. For Examples 235˜240, PyBOP and HOAt were used instead of EDC and HOBt.

TABLE 12

| Example | Starting amine (HNR₁R₂) | LC-MS, min. (m/Z) |
|---|---|---|
| 200 | 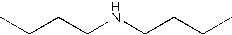 | 4.07 (435.1) |
| 201 | 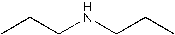 | 3.77 (407.1) |
| 202 | 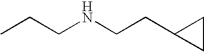 | 3.81 (419.1) |
| 203 | 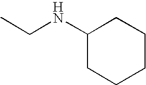 | 3.94 (433.1) |
| 204 | 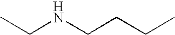 | 3.78 (407.1) |
| 205 | 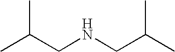 | 4.02 (435.1) |
| 206 | 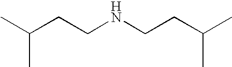 | 4.30 (463.1) |
| 207 | 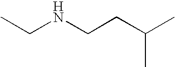 | 3.91 (421.1) |
| 208 | 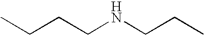 | 3.92 (421.1) |
| 209 | 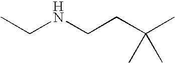 | 4.02 (435.1) |
| 210 | 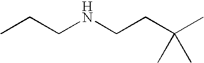 | 4.14 (449.1) |
| 211 | 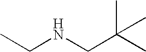 | 3.86 (421.1) |
| 212 | 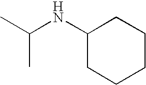 | 4.10 (447.1) |
| 213 | 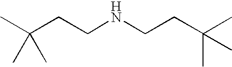 | 4.43 (491.2) |
| 214 | 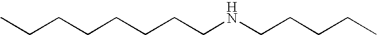 | 4.70 (505.2) |
| 215 | 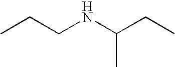 | 3.84 (421.1) |
| 216 | 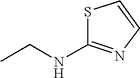 | 3.64 (434.0) |
| 217 | 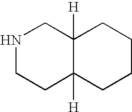 | 3.95 (445.1) |
| 218 | 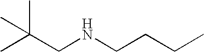 | 4.14 (449.1) |
| 219 | 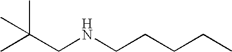 | 4.31 (463.1) |
| 220 | 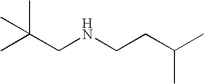 | 4.28 (463.2) |
| 221 | 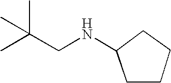 | 4.23 (461.1) |
| 222 | 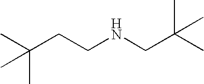 | 4.40 (477.2) |
| 223 | 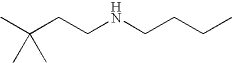 | 4.29 (463.2) |
| 224 | 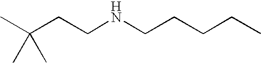 | 4.45 (477.2) |
| 225 | 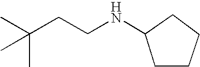 | 4.33 (475.1) |
| 226 | 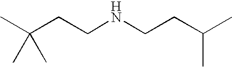 | 4.41 (477.2) |
| 227 | 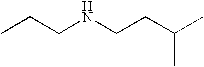 | 4.22 (449.2) |
| 228 | 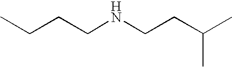 | 4.09 (435.2) |
| 229 | 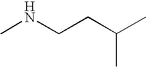 | 4.25 (449.3) |
| 230 | 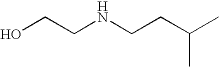 | 3.56 (437.2) |
| 231 | 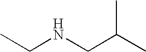 | 3.79 (407.3) |
| 232 | 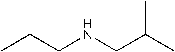 | 3.95 (421.3) |
| 233 | 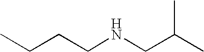 | 4.09 (435.4) |
| 234 | 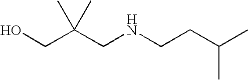 | 4.01 (479.4) |
| 235 | 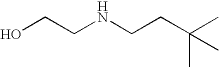 | 3.64 (451.3) |
| 236 | 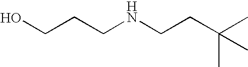 | 3.71 (443.3) |
| 237 | 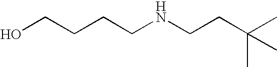 | 3.73 (479.3) |
| 238 | 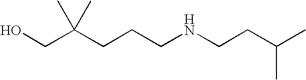 | 3.96 (507.3) |
| 239 | 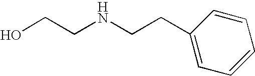 | 2.98 (475.3) |
| 240 | 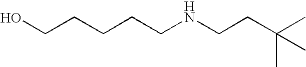 | 3.79 (493.4) |

### EXAMPLE 241

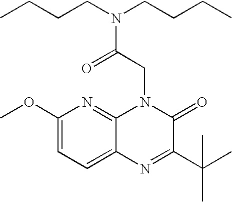

### N,N-Dibutyl-2-(2-tert-butyl-6-methoxy-3-oxopyrido[2,3-h]pyrazin-4(3H)-yl)acetamide

The title compound was prepared from (2-tert-butyl-6-methoxy-3-oxopyrido[2,3-b]pyrazin-4(3H)-yl)acetic acid from Preparative Example 50 using method described in Example 1. LC-MS: 4.30 min. (m/Z 403.4).

### EXAMPLE 242

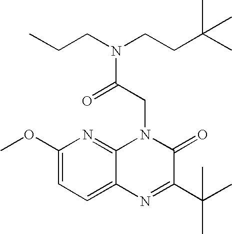

### 2-(2-tert-Butyl-6-methoxy-3-oxopyrido[2,3-b]pyrazin-4(3H)-yl)-N-(3,3-dimethylbutyl)-N-propylacetamide

The title compound was prepared from (2-tert-butyl-6-methoxy-3-oxopyrido[2,3-b]pyrazin-4(3H)-yl)acetic acid from Preparative Example 50 using method described in Example 1. Following an aqueous work-up using EtOAc, the crude product was purified on SGC and re-crystallized from 5:1 hexanes and EtOAc. LC-MS: 4.39 nm in. (m/Z 417.1).

### EXAMPLES 243˜256

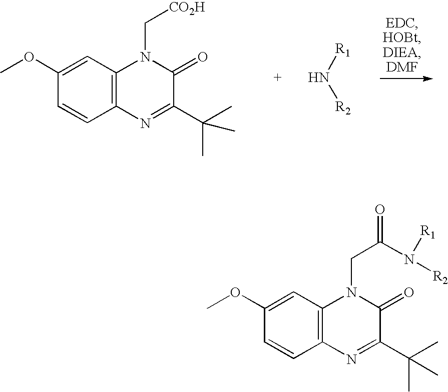

The following compounds in Table 13 were prepared using the method described in Example 1 using (2-tert-butyl-6-methoxy-3-oxopyrido[2,3-b]pyrazin-4(3H)-yl)acetic acid and the amine listed in Table 13.

TABLE 13

| Example | Starting amine (HNR₁R₂) | LC-MS, min. (m/Z) |
|---|---|---|
| 243 | 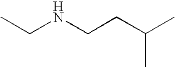 | 4.12 (389.4) |
| 244 | 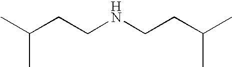 | 4.52 (431.2) |
| 245 | 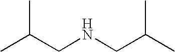 | 4.28 (403.1) |
| 246 | 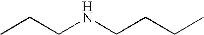 | 4.16 (389.2) |
| 247 | 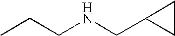 | 4.06 (387.3) |
| 248 | 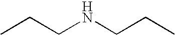 | 4.04 (375.3) |
| 249 | 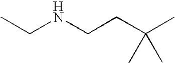 | 4.33 (403.3) |
| 250 | 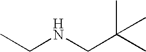 | 4.17 (389.4) |
| 251 | 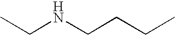 | 4.05 (375.3) |
| 252 | 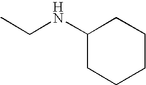 | 4.17 (401.2) |
| 253 | 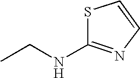 | 3.94 (402.0) |
| 254 | 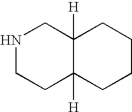 | 4.16 (413.1) |
| 255 | 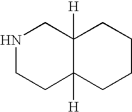 | 4.19 (413.1) |
| 256 | 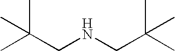 | 4.46 (431.1) |

### EXAMPLE 257

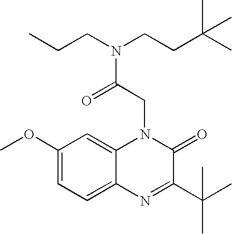

### 2-(3-tert-Butyl-7-methoxy-2-oxoquinoxalin-1(2H)-yl)-N-(3,3-dimethylbutyl)-N-propylacetamide

The title compound was prepared from (3-tert-Butyl-7-methoxy-2-oxoquinoxalin-1(2H)-yl)acetic acid from Preparative Example 51 using method described in Example 1. Following an aqueous work-up using EtOAc, the crude product was purified on SGC and re-crystallized from 4:1 hexanes and EtOAc. LC-MS: 4.36 min. (m/Z 416.1). ¹H NMR(CDC₃OD, 500 MHz) δ: 7.71 (d, 8.9 Hz, 1H), 6.94 (dd, 2.3 & 8.7 Hz, 1H), 6.62 & 6.58 (d, 2.2 Hz, 1H), 5.15 & 5.18 (s, 2H), 3.866 & 3.870 (s, 3H), 3.33˜3.56 (m, 4H), 1.48˜1.85 (m, 4H), 1.45 (s, 9H), 1.08 & 0.91 (t, 7.2 Hz, 3H), 1.05 & 0.93 (s, 9H).

### EXAMPLES 258-290

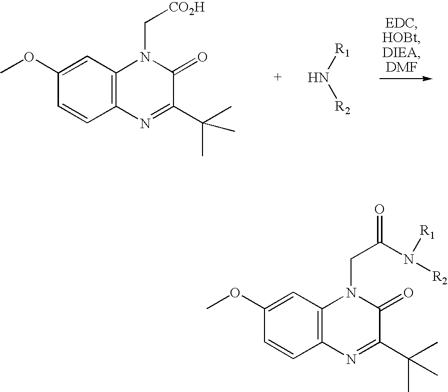

The following compounds in Table 14 were prepared using the method described in Example 1 using ((3-tert-Butyl-7-methoxy-2-oxoquinoxalin-1(2H)-yl)acetic acid and the amine listed in Table 14. Example 287 was obtained as the side-product during the preparation of Example 286.

TABLE 14

| Ex- | | |
|---|---|---|
| am- | | LC-MS, |
| ple | Starting amine (HNR₁R₂) | min. (m/Z) |
| 259 | 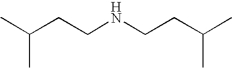 | 4.50 (430.3) |
| 260 | 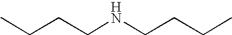 | 4.28 (402.3) |
| 261 | 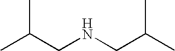 | 4.24 (402.3) |
| 262 | 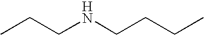 | 4.13 (388.3) |
| 263 | 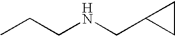 | 4.01 (386.3) |
| 264 | 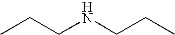 | 3.97 (374.3) |
| 265 | 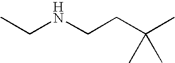 | 4.23 (402.3) |
| 266 | 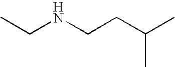 | 4.12 (388.3) |
| 267 | 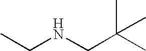 | 4.09 (388.3) |
| 268 | 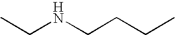 | 3.98 (374.3) |
| 269 | 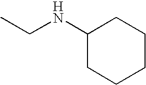 | 4.14 (400.3) |
| 270 | 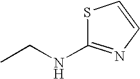 | 3.92 (401.2) |
| 271 | 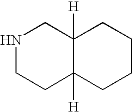 | 4.08 (412.1) |
| 272 | 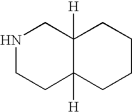 | 4.12 (412.1) |
| 273 | 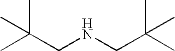 | 4.35 (430.2) |
| 274 | 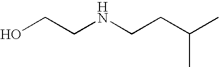 | 3.65 (404.2) |
| 275 | 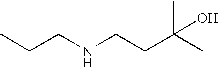 | 3.55 (418.4) |
| 276 | 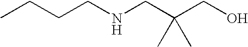 | 3.93 (432.4) |
| 277 | 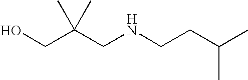 | 4.00 (446.4) |
| 278 | 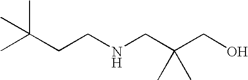 | 4.18 (460.4) |
| 279 | 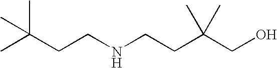 | 4.09 (474.4) |
| 280 | 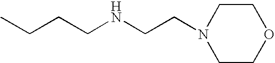 | 3.18 (459.4) |
| 281 | 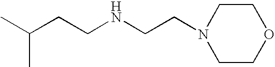 | 3.28 (473.4) |
| 282 | 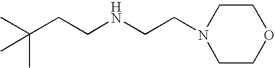 | 3.42 (487.5) |
| 283 | 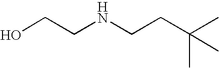 | 3.70 (418.4) |
| 284 | 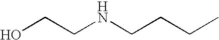 | 3.49 (390.4) |
| 285 | 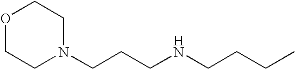 | 3.08 (473.3) |
| 286 | 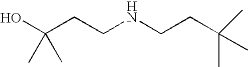 | 3.89 (460.4) |
| 287 | 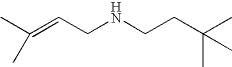 | 4.35 (442.4) |
| 288 | 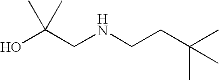 | 3.97 (446.5) |
| 289 | 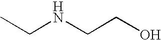 | 3.10 (362.4) |
| 290 | 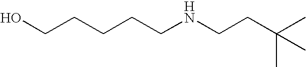 | 3.81 (460.1) |

### EXAMPLE 291

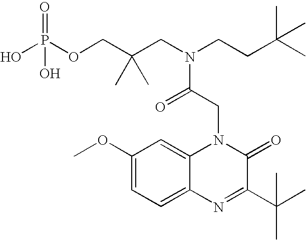

### 3-[[(3-tert-Butyl-7-methoxy-2-oxoquinoxalin-1(2H)-yl)acetyl](3,3-dimethylbutyl)amino]-2,2-dimethylpropyl dihydrogen phosphate

### Step A. Di-tert-butyl 3-[(3,3-dimethylbutyl)amino]-2,2-dimethylpropyl phosphate

Treat a solution of 100 mg 3-[(3,3-dimethylbutyl)amino]-2,2-dimethylpropan-1-ol and 156 mg di-tert-butyl diethylamidophosphite in 0.6 mL anhydrous DCM with 3.5 mL of 0.45 M tetrazole in MeCN at room temperature overnight. Cool half of this mixture to 40° C. and add a solution of 140 mg 72% mCPBA in 2.5 mL DCM. Remove the cooling bath and let the reaction mixture warm up to room temperature. LC-MS showed desired oxidation product. Treat the other half of the original reaction in the same manner. Combine the two halves of the reaction and quench with 10 mL 10% NaHCO₃ solution and adjust the pH to ˜10 with 5 N NaOH solution. Separate the layers, extract the aqueous layer with DCM (2×20 mL), wash the organic extract with saturated brine, dry over anhydrous Na₂SO₄, and evaporate to give crude title compound, which was used in the next step without further purification. LC-MS: 3.02 min. (m/Z 380.3, 324.4, 268.2). ¹H NMR (CDCl₃, 500 MHz) δ: 3.74 (d, 4.6 Hz, 1H), 2.62 (m, 1H), 2.50 (s, 2H), 1.51 (s, 9H), 1.50 (s, 18H), 1.42 (m, 2H), 0.97 (s, 6H), 0.91 (s, 9H).

### Step B. Di-tert-butyl 3-[[(3-tert-butyl-7-methoxy-2-oxoquinoxalin-1(2H)-yl)acetyl](3,3-dimethylbutyl)amino]-2,2-dimethylpropyl phosphate

The title compound was prepared from (2-tert-butyl-6-methoxy-3-oxopyrido[2,3-b]pyrazin-4(3H)-yl)acetic acid and di-tert-butyl 3-[(3,3-dimethylbutyl)amino]-2,2-dimethylpropyl phosphate using method described in Example 1. LC-MS: 4.56 min. (m/Z 540.3, 674.3). ¹H NMR (CDCl₃, 500 MHz) δ: 7.78 (d, 8.9 Hz, 1H), 6.91 (dd, 2.3 & 8.9 Hz, 1H), 6.47 (d, 2.3 Hz, 1H), 5.11 (s, 2H), 3.87 (s, 3H), 3.77 (d, 4.6 Hz, 2H), 3.49˜3.57 (m, 2H), 3.35 (br s, 2H), 1.68˜1.71 (m, 2H), 1.50 (s, 18H), 1.47 (s, 9H), 0.98 (s, 9H), 0.90 (s, 6H).

### Step C. 3-[[(3-tert-Butyl-7-methoxy-2-oxoquinoxalin-1(2H)-yl)acetyl](3,3-dimethylbutyl)amino]-2,2-dimethylpropyl dihydrogen phosphate

Dissolve 40 mg of di-tert-butyl 3-[[(3-tert-butyl-7-methoxy-2-oxoquinoxalin-1(2H)-yl)acetyl](3,3-dimethylbutyl)amino]-2,2-dimethylpropyl phosphate in 1 mL dioxane and treat it with 2 mL 4 N HCl in dioxane at room temperature. After one hour, HPLC and LC-MS showed no SM. This reaction mixture was purified on RP-HPLC using 35-100% MeCN with 0.1% TFA Combine pure product fractions, evaporate, and lyophilize to give the title compound. LC-MS: 3.63 min. (m/Z 268.3, 540.3). ¹H NMR (CD₃CN, 500 MHz) showed about 5:1 ratio of two rotomers. Major isomer: 7.67 (d, 9.0 Hz, 1H), 6.92 (dd, 9.0 & 2.3 Hz, 1H), 6.57 (d, 2.3 Hz, 1H), 5.11 (s, 2H), 3.86 (s, 3H), 3.62 (br s, 2H), 3.53˜3.57 (m, 2), 3.27 (br s, 2H), 1.67˜1.71 (m, 2H), 3.42 (s, 9H), 1.01 (s, 9H), 0.94 (s, 6H).

### EXAMPLE 292

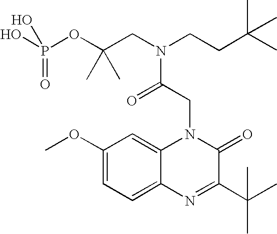

### 2-[[(3-tert-Butyl-7-methoxy-2-oxoquinoxalin-1(2H)-yl)acetyl](3,3-dimethylbutyl)amino]-1,1-dimethylethyl dihydrogen phosphate

### Step A. Benzyl 2-[(3,3-dimethylbutyl)amino]-1,1-dimethylethyl hydrogen phosphate

The title compound was prepared from 1-[(3,3-dimethylbutyl)amino]-2-methylpropan-2-ol and dibenzyl diethylamidophosphite using the procedure described in Step A Example 291, purified on RP-HPLC, and isolated as TFA salt. LC-MS: 2.42 min. (m/Z 344.1). ¹H NMR (CDCl₃, 500 MHz) δ: 8.69 (br s, 1H), 7.33˜7.40 (m, 5H), 5.03 (d, 7.4 Hz, 2H), 3.13˜3.14 (m, 2H), 3.02˜3.05 (m, 2H), 1.62˜1.65 (m, 2H), 1.51 (s, 6H), 0.91 (s, 9H).

### Step B. Benzyl 2-[[(3-tert-butyl-7-methoxy-2-oxoquinoxalin-1(2H)-yl)acetyl](3,3-dimethylbutyl)amino]-1,1-dimethylethyl hydrogen phosphate

The title compound was prepared from (2-tert-butyl-6-methoxy-3-oxopyrido[2,3-b]pyrazin-4(3H)-yl)acetic acid and benzyl 2-[(3,3-dimethylbutyl)amino]-1, —dimethylethyl hydrogen phosphate using procedure described in Example 1. LC-MS: 4.01 min. (m/Z 616.2, 428.3).

### Step C. 2-[[(3-tert-Butyl-7-methoxy-2-oxoquinoxalin-1(2H)-yl)acetyl](3,3-dimethylbutyl)amino]-1,1-dimethylethyl dihydrogen phosphate

Treat a mixture of 23.1 mg benzyl 2-[[(3-tert-butyl-7-methoxy-2-oxoquinoxalin-1(2H)-yl)acetyl](3,3-dimethylbutyl)amino]-1,1-dimethylethyl hydrogen phosphate and 2.0 mg 10% Pd/C in 1 mL MeOH with hydrogen balloon for 3.25 hours. The catalyst was removed by filtration and solvent removed under reduced pressure. The residue was purified on RP-HPLC using 40˜80% MeCN with 0.05% TFA Combine pure product fractions and lyophilize to give the title compound. LC-MS: 3.54 min. (m/Z 526).

### EXAMPLE 293

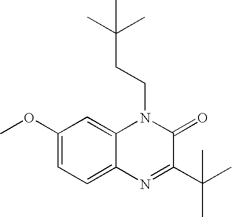

### 3-tert-Butyl-1-(3,3-dimethylbutyl)-7-methoxyquinoxalin-2(1H)-one

A solution of 21.3 mg 3-tert-Butyl-7-methoxyquinoxalin-2(1H)-one and 21.5 mg 1-bromo-3,3-dimethylbutane in 0.5 mL DMF was treated with 35.8 mg cesium carbonate at 46° C. for 3 days. The title compound was separated from slower-eluting isomeric 2-tert-butyl-3-(3,3-dimethylbutoxy)-6-methoxyquinoxaline on RP-HPLC. LC-MS: 4.55 min. (m/Z 317.2).

### EXAMPLE 294

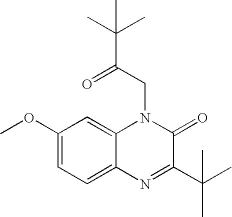

### 3-tert-Butyl-1-(3,3-dimethyl-2-oxobutyl)-7-methoxyquinoxalin-2(1H)-one

A solution of 47 mg 3-tert-Butyl-7-methoxyquinoxalin-2(1H)-one and 43 mg 1-bromo-3,3-dimethylbutan-2-one in 2 mL DMF was treated with 79 mg cesium carbonate at room temperature. The title compound was separated from minor slower-eluting isomeric 1-[(3-tert-butyl-7-methoxyquinoxalin-2-yl)oxy]-3,3-dimethylbutan-2-one on RP-HPLC. LC-MS: 4.02 min. (m/Z 331.3). ¹H NMR (CD₃OD, 500 MHz) δ: 7.73 (d, 8.9 Hz, 1H), 6.95 (dd, 2.5 & 8.9 Hz, 1H), 6.49 (d, 2.2 Hz, 1H), 5.40 (s, 2H), 3.86 (s, 3H), 1.44 (s, 9H), 1.36 (s, 9H). The structure of the title compound was further confirmed by NOE spectroscopy.

### EXAMPLES 295-301

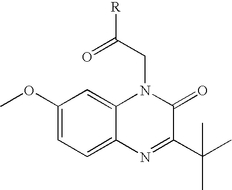

Examples 295-301 in Table 15 were prepared using the same method described in Example 294. Some of the bromoketones used were prepared using the method of Gaudry and Marguet (Org. Syn. Coll. Vol. 6, 193).

TABLE 15

| Example | Starting amine | LC-MS, min. (m/Z) |
|---|---|---|
| 295 | t-amyl | 4.04 (345.2) |
| 296 | 3-methyl-pent-3-yl | 4.18 (359.1) |
| 297 | i-propyl | 3.89 (317.1) |
| 298 | cyclohexyl | 4.16 (357.2) |
| 299 | phenyl | 3.95 (351.2) |
| 300 | cyclopentyl | 4.06 (343.3) |
| 301 | 1-adamantyl | 4.42 (409.4) |

### EXAMPLE 302

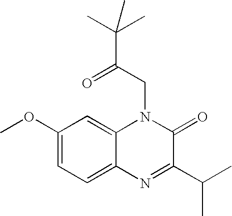

1-(3,3-Dimethyl-2-oxobutyl)-3-isopropyl-7-methoxyquinoxalin-2(11)-one

The title compound was prepared from 3-isopropyl-7-methoxyquinoxalin-2(1H)-one and 1-bromo-3,3-dimethylbutan-2-one using the method in Example 294. LC-MS: 3.56 min. (m/Z 317.1).

### EXAMPLES 303-315

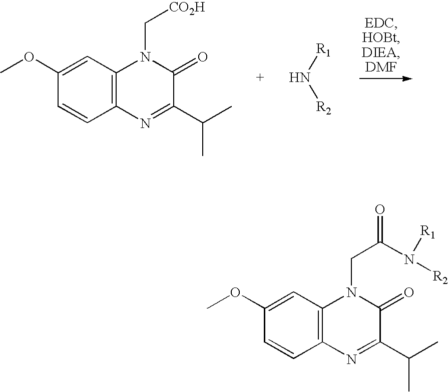

The following compounds in Table 16 were prepared using the method described in Example 1 using (3-isopropyl-7-methoxy-2-oxoquinoxalin-1(2H)-yl)acetic acid and the amine listed in Table 16.

TABLE 16

| Example | Starting amine )HNR₁R₂) | LC-MS, min. (m/Z) |
|---|---|---|
| 303 | 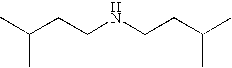 | 4.13 (416.1) |
| 304 | 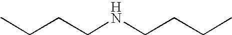 | 3.88 (388.1) |
| 305 | 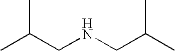 | 3.83 (388.1) |
| 306 | 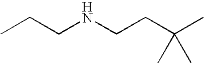 | 4.05 (402.2) |
| 307 | 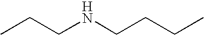 | 3.78 (374.2) |
| 308 | 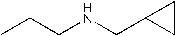 | 3.65 (372.2) |
| 309 | 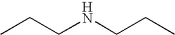 | 3.60 (360.2) |
| 310 | 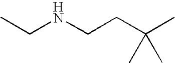 | 3.90 (388.2) |
| 311 | 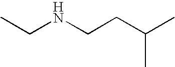 | 3.78 (374.2) |
| 312 | 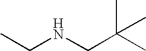 | 3.66 (374.1) |
| 313 | 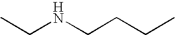 | 3.55 (360.1) |
| 314 | 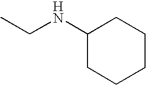 | 3.79 (386.2) |
| 315 | 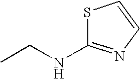 | 3.57 (387.1) |

### EXAMPLE 316

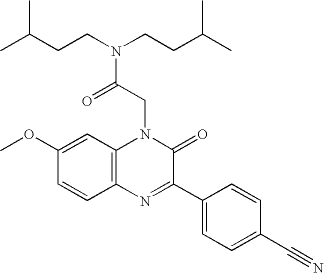

2-[3-(4-Cyanophenyl)-7-methoxy-2-oxoquinoxalin-1(2H)-yl]-N,N-bis(3-methylbutyl)acetamide

### Step A. tert-Butyl [3-(4-cyanophenyl)-7-methoxy-2-oxoquinoxalin-1(2H)-yl]acetate

The title compound was prepared from tert-butyl N-(5-methoxy-2-nitrophenyl)glycinate and ethyl (4-cyanophenyl)(oxo)acetate using method described in Preparative Example 51 Method B Step B. LC-MS: 3.75 min. (m/Z 392.0). ¹H NMR (CD₃OD, 500 MHz) δ: 8.49 (dd, 1.8 & 6.6 Hz, 2H), 7.89 (d, 9.0 Hz, 1HT), 7.82 (dd, 1.8 & 6.8 Hz, 2H), 7.08 (dd, 2.3 & 8.3 Hz, 1H), 6.82 (d, 2.6Hz, 1H), 5.11 (s, 2H), 3.95 (s, 3H).

### Step B. [3-(4-Cyanophenyl)-7-methoxy-2-oxoquinoxalin-1(2H)-yl]acetic acid

The title compound was prepared from tert-butyl [3-(4-cyanophenyl)-7-methoxy-2-oxoquinoxalin-1(2H)-yl]acetate using method described in Preparative Example 51

### Step C. 2-[3-(4-Cyanophenyl)-7-methoxy-2-oxoquinoxalin-1(2H)-yl]-N,N-bis(3-methylbutyl)acetamide

The title compound was prepared from [3-(4-cyanophenyl)-7-methoxy-2-oxoquinoxalin-1(2H)-yl]acetic acid using method described Example 1. LC-MS: 4.23 min. (m/Z 475.2). ¹H NMR (CD₃CN, 500 MHz) δ: 8.47 (m, 2H), 8.31 (m, 3H), 7.03 (dd, 2.5 & 8.9 Hz, 1H), 6.64 (d, 2.5 Hz, 1H), 5.15 (s, 2H), 3.44˜3.48 (m, 2H), 3.35˜3.38 (m, 2H), 1.65˜1.72 (m, 2H), 1.53˜1.58 (m, 1H), 1.41˜1.46 (m, 2H), 1.01 (d, 6.2 Hz, 6H), 0.90 (d, 6.6 Hz, 6H).

### EXAMPLES 317-333

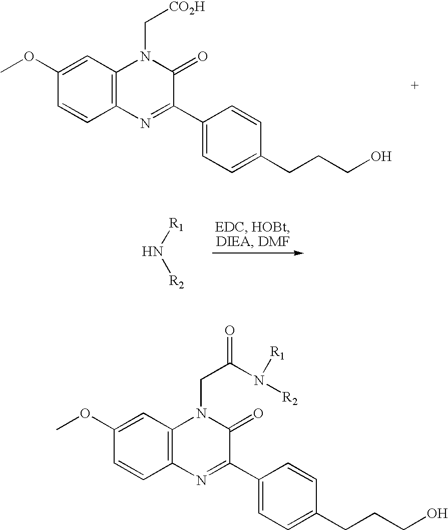

The following compounds in Table 17 were prepared using the method described in Example 1 using [3-[4-(3-Hydroxypropyl)phenyl]-7-methoxy-2-oxoquinoxalin-1(2H)-yl]acetic acid and the amine listed in Table 17.

TABLE 17

| Example | Starting amine (HNR₁R₂) | LC-MS, min. (m/Z) |
|---|---|---|
| 317 | 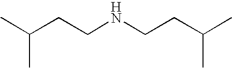 | 4.09 (508.2) |
| 318 | 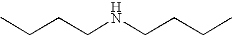 | 3.84 (480.1) |
| 319 | 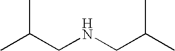 | 3.80 (480.1) |
| 320 | 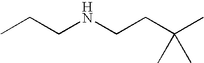 | 3.93 (494.2) |
| 321 | 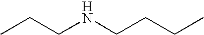 | 3.69 (466.1) |
| 322 | 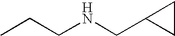 | 3.58 (464.1) |
| 323 | 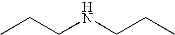 | 3.55 (452.1) |
| 324 | 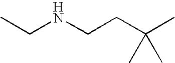 | 3.80 (480.2) |
| 325 | 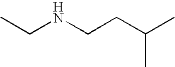 | 3.69 (466.2) |
| 326 | 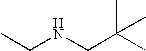 | 3.66 (466.2) |
| 327 | 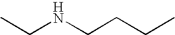 | 3.55 (452.2) |
| 328 | 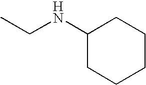 | 3.71 (478.2) |
| 329 | 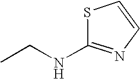 | 3.61 (479.2) |
| 330 | 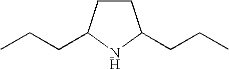 | 4.10 (506.24) |
| 331 | 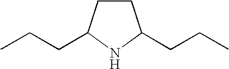 | 4.12 (506.25) |
| 332 | 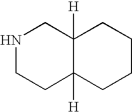 | 3.91 (490) |
| 333 | 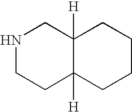 | 3.84 (490.25) |

### EXAMPLE 334

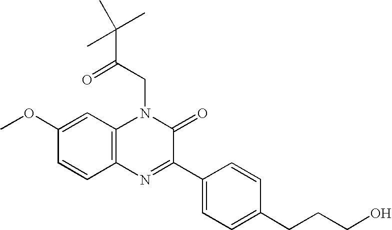

### 1-(3,3-Dimethyl-2-oxobutyl)-3-[4-(3-hydroxypropyl)phenyl]-7-methoxyquinoxalin-2(1H)-one

The title compound was prepared from 3-[4-(3-hydroxypropyl)phenyl]-7-methoxyquinoxalin-2(1H)-one and 1-bromo-3,3-dimethylbutan-2-one using method described in Example 294. LC-MS: 3.50 min. (m/Z 409.1). ¹H NMR (CDCl₃, 500 MHz) δ: 8.16˜8.17 (m, 2H), 7.90 (d, 8.9 Hz, 1H), 7.31 (d, 8.2 Hz, 2H), 6.97 (dd, 2.3 & 8.9 Hz, 1H), 6.34 (d, 2.5 Hz, 1H), 5.32 (s, 2H), 3.90 (s, 3H), 3.70 (t, 6.4 Hz, 2H), 2.78 (t, 7.4 Hz, 2M), 1.94˜1.97 (m, 2H), 1.40 (s, 9H).

### EXAMPLES 335˜333

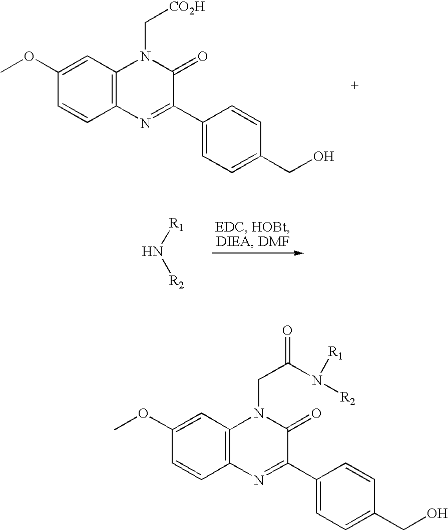

The following compounds in Table 18 were prepared using the method described in Example 1 using [[3-[4-(hydroxymethyl)phenyl]-7-methoxy-2-oxoquinoxalin-1(2H)-yl]acetic acid and the amine listed in Table 18.

TABLE 18

| Example | Starting amine (HNR₁R₂) | LC-MS, min. (m/Z) |
|---|---|---|
| 335 | 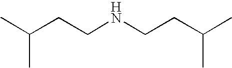 | 3.81 (480.3) |
| 336 | 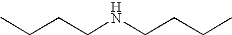 | 3.53 (452.2) |
| 337 | 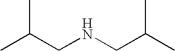 | 3.47 (452.2) |
| 338 | 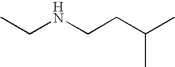 | 3.34 (438.3) |
| 339 | 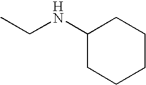 | 3.35 (450.3) |

### EXAMPLE 340

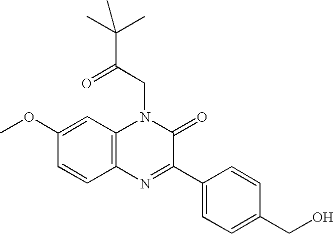

1-(3,3-dimethyl-2-oxobutyl)-3-[4-(hydroxymethyl)phenyl]-7-methoxyquinoxalin-2(1H)-one

The title compound was prepared from 3-[4-(hydroxymethyl)phenyl]-7-methoxyquinoxalin-2(1H)-one and 1-bromo-3,3-dimethylbutan-2-one using method described in Example 294. LC-MS: 3.15 min. (m/Z 381.2).

### EXAMPLE 341

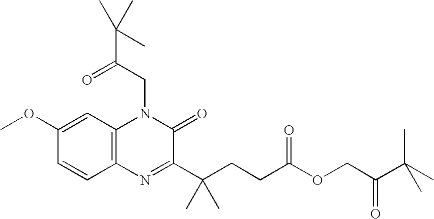

### 3,3-Dimethyl-2-oxobutyl 4-[4-(3,3-dimethyl-2-oxobutyl)-6-methoxy-3-oxo-3,4-dihydroquinoxalin-2-yl]-4-methylpentanoate

The title compound was prepared from 4-[4-(6-methoxy-3-oxo-3,4-dihydroquinoxalin-2-yl)phenyl]-4-methylpentanoic acid and 1.5 equiv. of 1-bromo-3,3-dimethylbutan-2-one using method described in Example 294. LC-MS: 4.02 min. (m/Z 487.2).

### EXAMPLE 342

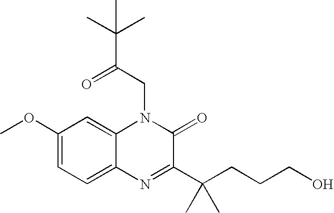

1-(3,3-Dimethyl-2-oxobutyl)-3-(4-hydroxy-1,1-dimethylbutyl)-7-methoxyquinoxalin-2(1H)-one

The title compound was prepared from 3-[4-(4-hydroxy-1,1-dimethylbutyl)phenyl]-7-methoxyquinoxalin-2(1H)-one and 1-bromo-3,3-dimethylbutan-2-one using method described in Example 294. LC-MS: 3.38 min. (m/Z 375.2).

### EXAMPLES 343-347

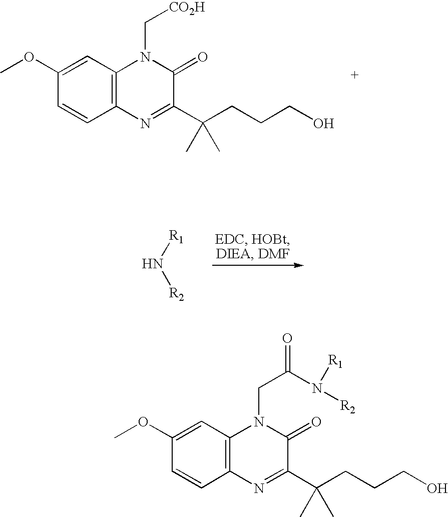

The following compounds in Table 19 were prepared using the method described in Example 1 using [3-(4-hydroxy-1,1-dimethylbutyl)-7-methoxy-2-oxoquinoxalin-1(2H)-yl]acetic acid and the amine listed in Table 19.

TABLE 19

| Example | Starting amine (HNR₁R₂) | LC-MS, min. (m/Z) |
|---|---|---|
| 343 | 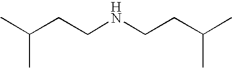 | 4.04 (474.5) |
| 344 | 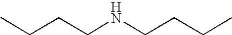 | 3.78 (446.4) |
| 345 | 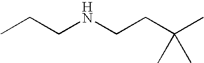 | 3.87 (460.4) |
| 346 | 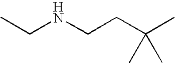 | 3.73 (446.4) |
| 347 | 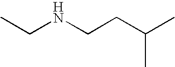 | 3.61 (432.4) |

### EXAMPLES 348-349

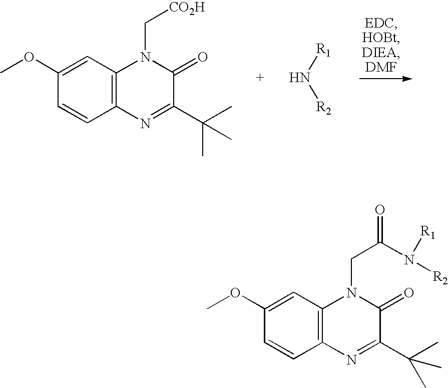

The following compounds in Table 20 were prepared using the method described in Example 1 using (3-tert-Butyl-7-methoxy-2-oxo-1,8-naphthyridin-1(2H)-yl)acetic acid and the amine listed in Table 20.

TABLE 20

| Example | Starting amine (HNR₁R₂) | LC-MS, min. (m/Z) |
|---|---|---|
| 348 | 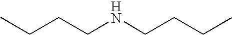 | 4.17 (402.1) |
| 349 | 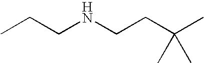 | 4.24 (416.1) |

### EXAMPLES 350

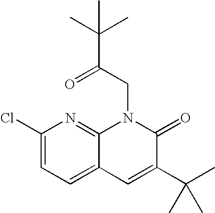

### 3-tert-Butyl-7-chloro-1-(3,3-dimethyl-2-oxobutyl)-1,8-naphthyridin-2(1H)-one

### Step A. 3-tert-Butyl-7-chloro-1,8-naphthyridin-2(1H)-one

The title compound was prepared by treating 14.5 mg 3-tert-butyl-1,8-naphthyridin-2(1H)-one 8-oxide with 1 mL thionyl chloride at room temperature overnight. It was separated from its 7-hydroxy and 4-chloro derivatives on RP-HPLC. LC-MS: 3.21 min. (n/Z 237.1). ¹H NMR (CDCl₃, 500 MHz) δ: 7.85 (d, 8.0 Hz, 1H), 7.64 (s, 1H), 7.22 (d, 8.0 Hz, 1H), 1.45 (s, 9H)

### Step A. 3-tert-Butyl-7-chloro-1-(3,3-dimethyl-2-oxobutyl)-1,8-naphthyridin-2(1H)-one

The title compound was prepared from 3-tert-butyl-7-chloro-1,8-naphthyridin-2(1H)-one and 1-bromo-3,3-dimethylbutan-2-one using method described in Example 294. LC-MS: 3.97 min. (m/Z 335.1).

### EXAMPLES 351

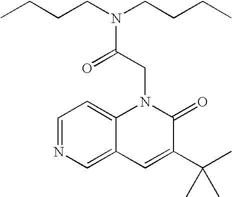

### N,N-Dibutyl-2-(3-tert-butyl-2-oxo-1,6-naphthyridin-1(2H)-yl)acetamide

### Step A. Methyl 2-[{4-[(2,2-dimethylpropanoyl)amino]pyridin-3-yl} (hydroxy)methyl]-3,3-dimethylbutanoate

The title compound was prepared from N-(3-formylpyridin-4-yl)-2,2-dimethylpropanamide and methyl 3-methylbutanoate using the method of Turner (J. Org. Chem. 55, 4744, 1990). LC-MS: 2.64 and 2.78 min. (m/Z 337.2). ¹H NMR of two diastereomers (CDCl₃, 500 MHz) δ: 9.68 & 9.60 (br s, 1H), 8.43 & 8.28 (d, 5.8, 1H), 8.32 (d, 5.7 Hz, 1H), 8.23 & 8.05 (br s, 1H), 5.10˜5.16 (m, 1H), 3.78 & 3.30 (s, 3H), 3.00 & 2.86 (d, 5.1 or 10.8 Hz, 1H), 1.35 & 1.34 (s, 9H), 1.21 & 0.96 (s, 9H).

### Step B. 3-tert-Butyl-1,6-naphthyridin-2(1H)-one

The title compound was prepared from methyl 2-[{4-[(2,2-dimethylpropanoyl)amino]pyridin-3-yl}(hydroxy)methyl]-3,3-dimethylbutanoate using the method described in Preparative Example 60 Step B by heating in a microwave reactor at 160° C. for 2 hours. LC-MS: 1.75 min. (m/Z 203.1). ¹H NMR (CDCl₃, 500 MHz) δ: 8.83 (s, 1H), 8.54 (d, 5.7 Hz, 1H), 7.75 (s, 1H), 7.18 (d, 5.7 Hz, 1H), 1.51 (s, 9H).

### Step C. Methyl (3-tert-butyl-2-oxo-1,6-naphthyridin-1(2H)-yl)acetate

The title compound was prepared from 3-tert-butyl-1,6-naphthyridin-2(1H)-one and methyl bromoacetate using the method described in Preparative Example 57 Step G. It was separated from the slower-eluting isomeric methyl [(3-tert-butyl-1,6-naphthyridin-2-yl)oxy]acetate on RP-HPLC. LC-MS: 2.18 min. (m/Z 275.1). ¹H NMR (CDC₃OD, 500 MHz) δ: 9.20 (s, 1H), 8.67 (d, 7.1 Hz, 1H), 8.10 (s, 1H), 7.89 (d, 7.1 Hz, 1H), 5.21 (d, 2H), 3.82 (s, 3H), 1.43 (s, 9H).

### Step D. (3-tert-Butyl-2-oxo-1,6-naphthyridin-1(2H)-yl)acetic acid

The title compound was prepared from methyl (3-tert-butyl-2-oxo-1,6-naphthyridin-1(2H)-yl)acetate using the method described in Preparative Example 57 Step H. LC-MS: 1.92 min. (m/Z 261.2).

### Step E. N,N-Dibutyl-2-(3-tert-butyl-2-oxo-1,6-naphthyridin-1(2H)-yl)acetamide

The title compound was prepared using the method described in Example 1 using (3-tert-butyl-2-oxo-1,6-naphthyridin-1(2H)-yl)acetic acid and dibutylamine. LC-MS: 3.23 min. (m/Z 372.2).

### EXAMPLES 352

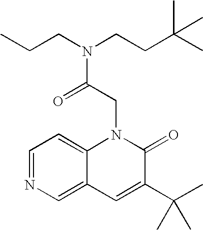

### 2-(3-tert-Butyl-2-oxo-1,6-naphthyridin-1(2H)-yl)-N-(3,3-dimethylbutyl)-N-propylacetamide

The title compound was prepared using the method described in Example 1 using (3-tert-butyl-2-oxo-1,6-naphthyridin-1(2H)-yl)acetic acid and 3,3-dimethyl-N-propylbutan-1-amine. LC-MS: 3.33 min. (m/Z 386.2).

### Functional Assays

### A. Maxi-K Channel

The identification of inhibitors of the Maxi-K channel can be accomplished using Aurora Biosciences technology, and is based on the ability of expressed Maxi-K channels to set cellular resting potential after transient transfection of both α and β subunits of the channel in TsA-201 cells. In the absence of inhibitors, cells display a hyperpolarized membrane potential, negative inside, close to E_{K} (−80 mV) which is a consequence of the activity of the Maxi-K channel. Blockade of the Maxi-K channel will cause cell depolarization. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)). Oxanol is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, to a decrease in FRET. Thus, the ratio change (donor/acceptor) increases after membrane depolarization.

Transient transfection of the Maxi-K channel in TsA-201 cells can be carried out as previously described (Hanner et al. (1998) J. Biol. Chem. 273, 16289-16296) using FUGENE6™ as the transfection reagent. Twenty four hours after transfection, cells are collected in Ca²⁺—Mg²⁺-free Dulbecco's phosphate-buffered saline (D-PBS), subjected to centrifugation, plated onto 96-well poly-d-lysine coated plates at a density of 60,000 cells/well, and incubated overnight. The cells are then washed 1× with D-PBS, and loaded with 100 μl of 4 μM CC₂DMPE-0.02% pluronic-127 in D-PBS. Cells are incubated at room temperature for 30 min in the dark. Afterwards, cells are washed 2× with D-PBS and loaded with 100 μl of 6 μM DiSBAC₂(3) in (mM): 140 NaCl, 0.1 KCl, 2 CaCl₂, 1 MgCl₂, 20 Hepes-NaOH, pH 7.4, 10 glucose. Test compounds are diluted into this solution, and added at the same time. Cells are incubated at room temperature for 30 min in the dark.

Plates are loaded into a voltage/ion probe reader (VIPR) instrument, and the fluorescence emission of both CC₂DMPE and DiSBAC₂(3) are recorded for 10 sec. At this point, 1001 of high-potassium solution (mM): 140 KCl, 2 CaCl₂, 1 MgCl₂, 20 Hepes-KOH, pH 7.4, 10 glucose are added and the fluorescence emission of both dyes recorded for an additional 10 sec. The ratio CC₂DMPE/DiSBAC₂(3), before addition of high-potassium solution equals 1. In the absence of any inhibitor, the ratio after addition of high-potassium solution varies between 1.65-2.0. When the Maxi-K channel has been completely inhibited by either a known standard or test compound, this ratio remains at 1. It is possible, therefore, to titrate the activity of a Maxi-K channel inhibitor by monitoring the concentration-dependent change in the fluorescence ratio.

The compounds of this invention were found to cause concentration-dependent inhibition of the fluorescence ratio with IC₅₀'s in the range of about 5 nM to about 500 μM, more preferably from about 5 nM to about 20 nM.

B. Electrophysiological Assays of Compound Effects on High-Conductance Calcium-Activated potassium channels

### Human Non-Pigmented Ciliary Epithelial Cells

The activity of high-conductance calcium-activated potassium (maxi-K) channels in human non-pigmented ciliary epithelial cells was determined using electrophysiological methods. Currents through maxi-K channels were recorded in the inside-out configuration of the patch clamp technique, where the pipette solution faces the extracellular side of the channel and the bath solution faces the intracellular side. Excised patches contained one to about fifty maxi-K channels. Maxi-K channels were identified by their large single channel conductance (250-300 pS), and by sensitivity of channel gating to membrane potential and intracellular calcium concentration. Membrane currents were recorded using standard electrophysiological techniques. Glass pipettes (Garner 7052) were pulled in two stages with a Kopf puller (model 750), and electrode resistance was 1-3 megohms when filled with saline. Membrane currents were recorded with EPC9 (HEKA Instruments) or Axopatch 1D (Axon Instruments) amplifiers, and digital conversion was done with ITC-16 interfaces (Instrutech Corp). Pipettes were filled with (mM); 150 KCl, 10 Hepes, 1 MgCl₂, 0.01 CaCl₂, 3.65 KOH, pH 7.20. The bath (intracellular) solution was identical, except, in some cases, calcium was removed, 1 mM EGTA was added and 20 mM KCl was replaced with 20 mM KF to eliminate calcium to test for calcium sensitivity of channel gating. Drugs were applied to the intracellular side of the channel by bath perfusion.

Human non-pigmented ciliary epithelial cells were grown in tissue culture as described (Martin-Vasallo, P., Ghosh, S., and Coca-Prados, M., 1989, J. Cell. Physiol. 141, 243-252), and plated onto glass cover slips prior to use. High resistance seals (>1 Gohm) were formed between the pipette and cell surface, and inside out patches were excised. Maxi-K channels in the patch were identified by their gating properties; channel open probability increased in response to membrane depolarization and elevated intracellular calcium. In patches used for pharmacological analysis, removing intracellular calcium eliminated voltage-gated currents. Maxi-K currents were measured after depolarizing voltage steps or ramps that caused channel opening.

The compounds of this invention were applied to the intracellular side of the channel in appropriate concentrations (0.001 to 100 μM). The compounds reduced channel open probability, and this effect was reversed upon washout of compounds from the experimental chamber. The IC₅₀ for block of maxi-K channels under these conditions for the compounds of this invention ranged from about 0.2 nM to about 100 μM.

What is claimed is:

1. A compound of the structural formula I: 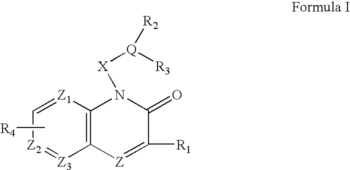
or a pharmaceutically acceptable salt, ester including phosphate, enantiomer, diastereomer or mixture thereof:
wherein,
Z, Z₁, Z₂, and Z₃ independently represent CH or N;
R and R^{y} independently represent hydrogen, or C₁₋₆ alkyl;
R₁ represents hydrogen or C₁₋₆ alkyl, CF₃, (CH₂)ₙC₃₋₁₀ cycloalkyl, (CH₂)ₙC₆₋₁₀ aryl, —(CH₂)ₙC₅₋₁₀ heteroaryl, C₁₋₆ alkoxy, OH, COR^{C}, said alkyl, cycloalkyl, aryl, heteroaryl, and alkoxy optionally substituted with 1-3 groups selected from R^{b};
X represents —(CHR₇)ₚ—, —(CR₇)ₚC(O)—;
Q represents N, CR^{y}, or O, wherein R₂ is absent when Q is O;
R₂ represents hydrogen, C₁₋₁₀ alkyl, C₂₋₁₀ hydroxylalkyl, C₁₋₆ alkylSR, —(CH₂)ₙO(CH₂)ₘOR, (CH₂)ₘOR, —(CH₂)ₙ(CHR₇)ₛ(CH₂)ₘC₁₋₆ alkoxy, —(CH₂)ₙ(CHR₇)(CH₂)ₘC₃₋₈ cycloalkyl, —(CH₂)ₙ(CHR₇)ₛ(CH₂)ₘC₃₋₁₀ heterocyclyl, —(CH₂)ₙC₅₋₁₀ heteroaryl, —N(R)₂, —COOR, or —(CH₂)ₙ(CHR₇)ₛ(CH₂)ₘC₆₋₁₀ aryl, said allyl, cycloalkyl, heterocyclyl, aryl or heteroaryl optionally substituted with 1-3 groups selected from R^{a};
R₃ represents hydrogen, C₁₋₁₀ alkyl, C₂₋₆ alkenyl, —(CH₂)ₙ(CHR₇)ₛ(CH₂)ₘC₃₋₈ cycloalkyl, —(CH₂)ₙ(CHR₇)ₛ(CH₂)ₘC₃₋₁₀ heterocyclyl, —(CH₂)ₙ(CHR₇)(CH₂)ₘC₅₋₁₀ heteroaryl, —(CH₂)ₙ(CHR₇)ₛ(CH₂)ₘCOOR, —(CH₂)ₙ(CHR₇)ₛ(CH₂)ₘC₆₋₁₀ aryl, —(CH₂)ₙ(CHR₇)ₛ(CH₂)ₘNHR₈, —(CH₂)ₙ(CHR₇)ₛ(CH₂)ₘN(R)₂, —(CH₂)ₙ(CHR₇)ₛ(CH₂)ₘN(R₈)₂, —(CH₂)ₙ(CHR₇)ₛ(CH₂)ₘNHCOOR, —(CH₂)ₙ(CHR₇)ₛ(CH₂)ₘN(R₈)CO₂R, —(CH₂)ₙ(CHR₇)ₛ(CH₂)ₘN(R₈)COR, —(CH₂)ₙ(CHR₇)ₛ(CH₂)ₘNHCOR, —(CH₂)ₙ(CHR₇)ₛ(CH₂)ₘCONH(R₈), aryl, —(CH₂)ₙ(CHR₇)ₛ(CH₂)ₘOR, —(CH₂)ₙC(R₇)₂(CH₂)ₘOR, CF₃, —(CH₂)ₙ(CHR₇)ₛ(CH₂)ₘSO₂R, —(CH₂)ₙ(CHR₇)ₛ(CH₂)ₘSO₂N(R)₂, —(CH₂)ₙ(CHR₇)ₛ(CH₂)ₘCON(R)₂, —(CH₂)ₙ(CHR₇)ₛ(CH₂)ₘCONHC(R)₃, —(CH₂)ₙCONHC(R)₂CO₂R, —(CH₂)ₙ(CHR₇)ₛ(CH₂)ₘCOR₈, nitro, cyano or halogen, said alkyl, cycloalkyl, alkoxy, heterocyclyl, aryl or heteroaryl optionally substituted with 1-3 groups of R^{a};
or, when Q is N, R₂ and R₃ taken together with the intervening N atom form a 4-10 membered heterocyclic ring optionally interrupted by 1-2 atoms of O, S, C(O) or NR, and optionally having 1-4 double bonds, and optionally substituted by 1-3 groups selected from R^{a};
or, when Q equals CR^{y}, R₂ and R₃ taken together with the intervening CR^{y} form a 4-10 membered carbocyclic or heterocyclic aromatic ring or fused ring optionally interrupted by 1-2 atoms of O, S, C(O) or NR, and optionally having 1-5 double bonds, and optionally substituted by 1-3 groups selected from R^{a};
R₄ represents hydrogen, C₁₋₆ alkoxy, halogen, cyano, OH, C₁₋₆ alkyl, COOR, SO₃H, C₁₋₆ alkylcarbonyl, S(O)_{q}R^{y}, —O(CH₂)ₙN(R)₂, —O(CH₂)ₙCO₂R, —OPO(OH)₂, CF₃, —N(R)₂, nitro, or C₁₋₆ alkylamino;
R₇ represents hydrogen, C₁₋₆ alkyl, —(CH₂)ₙCOOR or —(CH₂)ₙN(R)₂,
R₈ represents —(CH₂)ₙC₃₋₈ cycloalkyl, —(CH₂)_{n 3-10} heterocyclyl, C₁₋₆ alkoxy or —(CH₂)ₙC₅₋₁₀ heteroaryl, —(CH₂)ₙC₆₋₁₀ aryl said heterocyclyl, cycloalkyl, aryl or heteroaryl optionally substituted with 1-3 groups selected from R^{a};
R^{a} represents F, Cl, Br, I, CF₃, N(R)₂, NO₂, CN, —(CH₂)ₙCOR₈, —(CH₂)ₙCONHR₈, —(CH₂)ₙCON(R₈)₂, —O(CH₂)ₙCOOR, —NH(CH₂)ₙOR, —COOR, —OCF₃, —O—, —NHCOR, —SO₂R, —SO₂NR₂, —SR, (C₁-C₆ alkyl)O—, —(CH₂)ₙ—O—(CH₂)ₘOR, —(CH₂)ₙC₁₋₆ alkoxy, (aryl)O—, —OH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —(C₁-C₆ alkyl)-C₃₋₁₀ heterocyclyl-R_{w}, —(CH₂)ₙ—K—C(═K)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-K—C(═K)N(R)₂, —(CH₂)ₙSO₂R, —(CH₂)ₙSO₃H, —(CH₂)ₙPO(OR)₂, —(CH₂)ₙOPO(OR)₂, cyclohexyl, cyclopentyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, and COOR;
K independently represents CH, CH₂ or NH;
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R^{b} represents C₁₋₆ alkyl, —COOR, —SO₃R, CN, (CH₂)ₙOR, C(O)O(CH₂)ₙC(O)R, —OPO(OH)₂, —(CH₂)ₙC₆₋₁₀ aryl, or —(CH₂)ₙC₅₋₁₀ heteroaryl;
R^{c} represents hydrogen, C₁₋₆ alkyl, or —(CH₂)ₙC₆₋₁₀ aryl;
m is 0-3;
n is 0-3;
q is 0-2;
s is 0-1; and
p is 0-2.

2. A compound according to claim 1 wherein Q is N, X is —CR₇)ₚC(O)—, R₁ is C₁₋₆ alkyl, Z is N, Z₁, Z₂, and Z₃ are each CH, QR₂R₃ is a dialkylamine, hydroxydialkylamine or hydroxylamine and R₇ is hydrogen or C₁₋₆ alkyl, and all other variables are as originally described.

3. A compound according to claim 1 wherein R₂ and R₃ are taken together with the intervening N atom form a 4-10 membered heterocyclic carbon ring optionally interrupted by 1-2 atoms of O, S, C(O) or NR, and optionally having 1-4 double bonds, and optionally substituted by 1-3 groups selected from R^{a}

4. A compound according to claim 1 of structural formula II: 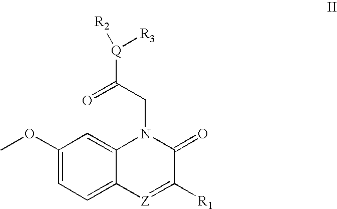
or a pharmaceutically acceptable salt, ester including phosphate, enantiomer, diastereomer or mixture thereof:
wherein,
Q is N;
Z is CH or N;
R₁ represents hydrogen or C₁₋₆ alkyl, (CH₂)ₙC₃₋₁₀ cycloalkyl, (CH₂)ₙC₆₋₁₀ aryl, C₁₋₆ alkoxy, said alkyl, cycloalkyl, aryl, heteroaryl and alkoxy optionally substituted with 1-3 groups selected from R^{b};
R₂ represents hydrogen, C₁₋₁₀ alkyl, C₂₋₁₀ hydroxylalkyl, (CH₂)ₘOR, —(CH₂)ₙ(CHR₇)ₛ(CH₂)ₘC₁₋₆ alkoxy, —(CH₂)ₙ(CHR₇)ₛ(CH₂)ₘC₃₋₈ cycloalkyl, —(CH₂)ₙ(CHR₇)ₛ(CH₂)ₘC₃₋₁₀ heterocyclyl, —(CH₂)ₙC₅₋₁₀ heteroaryl, or —(CH₂)ₙ(CHR₇)ₛ(CH₂)ₘC₆₋₁₀ aryl, said alkyl, cycloalkyl, heterocyclyl, aryl or heteroaryl optionally substituted with 1-3 groups selected from R^{a};
R₃ represents hydrogen, C₁₋₁₀ alkyl, —(CH₂)ₙ(CHR₇)ₛ(CH₂)ₘC₃₋₈ cycloalkyl, —(CH₂)ₙ(CHR₇)ₛ(CH₂)ₘC₃₋₁₀ heterocyclyl, —(CH₂)ₙ(CHR₇)ₛ(CH₂)ₘC₅₋₁₀ heteroaryl, or —(CH₂)ₙ(CHR₇)ₛ(CH₂)ₘC₆₋₁₀ aryl, said alkyl, cycloalkyl, alkoxy, heterocyclyl, aryl or heteroaryl optionally substituted with 1-3 groups of R^{a}; and all other variables are as described herein.

5. A compound according to claim 6 wherein R₁ is C₁₋₆ alkyl, (CH₂)ₙC₆₋₁₀ aryl, or (CH₂)ₙC₃₋₁₀ cycloalkyl, optionally substituted with 1 to 3 groups of R^{b} and R₂ and R₃ are independently hydrogen, C₁₋₁₀ alkyl, said alkyl, optionally substituted with 1-3 groups selected from R^{a}.

6. A compound which is selected from:
N,N-Dibutyl-2-(3-tert-butyl-7-methoxy-2-oxoquinolin-1(2H)-yl)acetamide,
2-(3-Tert-butyl-7-methoxy-2-oxoquinolin-1(2H)-yl)-N,N-bis(3-methylbutyl)acetamide,
2-(3-Tert-butyl-7-methoxy-2-oxoquinolin-1(2H)-yl)-N-(cyclopropylmethyl)-N-propylacetamide,
2-(3-Tert-butyl-7-methoxy-2-oxoquinolin-1(2H)-yl)-N,N-diisobutylacetamide,
2-(3-Tert-butyl-7-methoxy-2-oxoquinolin-1(2H)-yl)-N-(3,3-dimethylbutyl)-N-ethylacetamide,
2-(3-Tert-butyl-7-methoxy-2-oxoquinolin-1(2H)-yl)-N-(3,3-dimethylbutyl)-N-propylacetamide,
2-(3-Tert-butyl-7-methoxy-2-oxoquinolin-1(2H)-yl)-N-(2,2-dimethylpropyl)-N-ethylacetamide,
2-(3-Tert-butyl-7-methoxy-2-oxoquinolin-1(2H)-yl)-N-ethyl-N-(3-methylbutyl)acetamide,
2-(3-Tert-butyl-7-methoxy-2-oxoquinolin-1(2H)-yl)-N-(3-methylbutyl)-N-propylacetamide,
N-Butyl-2-(3-tert-butyl-7-methoxy-2-oxoquinolin-1(2H)-yl)-N-(3-methylbutyl)acetamide,
2-(3-Tert-butyl-7-methoxy-2-oxoquinolin-1(2H)-yl)-N-(2-hydroxyethyl)-N-(3-methylbutyl)acetamide,
2-(3-Tert-butyl-7-methoxy-2-oxoquinolin-1(2H)-yl)-N-isobutyl-N-propylacetamide,
N-butyl-2-(3-tert-butyl-7-methoxy-2-oxoquinolin-1(2H)-yl)-N-isobutylacetamide,
2-(3-Tert-butyl-7-methoxy-2-oxoquinolin-1(2H)-yl)-N-(3-hydroxy-2,2-dimethylpropyl)-N-(3-methylbutyl)acetamide,
2-(3-Tert-butyl-7-methoxy-2-oxoquinolin-1(2H)-yl)—N-(5-hydroxy-4,4-dimethylpentyl)-N-(3-methylbutyl)acetamide,
2-(3-Tert-butyl-7-methoxy-2-oxoquinolin-1(2H)-yl)-N-(3,3-dimethylbutyl)-N-(3-hydroxy-2,2-dimethylpropyl)acetamide,
2-(3-Tert-butyl-7-methoxy-2-oxoquinolin-1(2H)-yl)-N-(3,3-dimethylbutyl)-N-(2-hydroxy-2-methylpropyl)acetamide,
2-(3-Tert-butyl-7-methoxy-2-oxoquinolin-1(2H)-yl)-N-(5-hydroxypentyl)-N-(3-methylbutyl)acetamide,
2-(3-Tert-butyl-7-methoxy-2-oxoquinolin-1(2H)-yl)-N-(3,3-dimethylbutyl)-N-(5-hydroxypentyl)acetamide,
2-(3-tert-butyl-7-methoxy-2-oxoquinolin-1(2-yl)-N-(3,3-dimethylbutyl)-N-(2-hydroxy-1,1-dimethylethyl)acetamide,
3-Tert-butyl-1-(3,3-dimethyl-2-oxobutyl)-7-methoxyquinolin-2(1H)-one,
N,N-Dibutyl-2-(3-isopropyl-7-methoxy-2-oxoquinolin-1(2H)-yl)acetamide,
N,N-Diisobutyl-2-(3-isopropyl-7-methoxy-2-oxoquinolin-1(2H)-yl)acetamide,
1-(3,3-Dimethylbutyl)-N-ethyl-2-(3-isopropyl-7-methoxy-2-oxoquinolin-1(2H)-yl)acetamide,
N-(3,3-Dimethylbutyl)-2-(3-isopropyl-7-methoxy-2-oxoquinolin-1(2H)-yl)-N-propylacetamide,
2-(3-Isopropyl-7-methoxy-2-oxoquinolin-1(2H)-yl)-N-octyl-N-pentylacetamide,
N-Ethyl-2-(3-isopropyl-7-methoxy-2-oxoquinolin-1(2H)-yl)-N-1,3-thiazol-2-ylacetamide,
(±)-3-Isopropyl-7-methoxy-1-{2-[trans-octahydroisoquinolin-2-(1H)-yl]-2-oxoethyl} quinolin-2(1H)-one,
N,N-Bis(2,2-dimethylpropyl)-2-(3-isopropyl-7-methoxy-2-oxoquinolin-1(2H)-yl)acetamide,
2-(3-Isopropyl-7-methoxy-2-oxoquinolin-1(2H)-yl)-N-(3-methylbutyl)-N-propylacetamide,
N-Butyl-2-(3-isopropyl-7-methoxy-2-oxoquinolin-1(2H)-yl)-N-(3-methylbutyl)acetamide,
N-Isobutyl-2-(3-isopropyl-7-methoxy-2-oxoquinolin-1(2H)-yl)-N-propylacetamide,
N-Butyl-N-isobutyl-2-(3-isopropyl-7-methoxy-2-oxoquinolin-1(2H)-yl)acetamide,
N-(5-Hydroxy-4,4-dimethylpentyl)-2-(3-isopropyl-7-methoxy-2-oxoquinolin-1(2H)-yl)-N-(3-methylbutyl)acetamide,
N-(3,3-Dimethylbutyl)-N-(5-hydroxypentyl)-2-(3-isopropyl-7-methoxy-2-oxoquinolin-1(2H)-yl)acetamide,
N,N-Dibutyl-2-(3-cyclohexyl-7-methoxy-2-oxoquinolin-1(2H)-yl)acetamide,
2-(3-Cyclohexyl-7-methoxy-2-oxoquinolin-1(2H)-yl)-N,N-dipropylacetamide,
2-(3-Cyclohexyl-7-methoxy-2-oxoquinolin-1(2H)-yl)-N-(cyclopropylmethyl)-N-propylacetamide,
N-Cyclohexyl-2-(3-cyclohexyl-7-methoxy-2-oxoquinolin-1(2H)-yl)-1-ethylacetamide,
2-(3-Cyclohexyl-7-methoxy-2-oxoquinolin-1(2H)-yl)-N,N-diisobutylacetanide,
2-(3-Cyclohexyl-7-methoxy-2-oxoquinolin 1(2H)-yl)-N,N-bis(3-methylbutyl)acetamide,
2-(3-Cyclohexyl-7-methoxy-2-oxoquinolin-1(2H)-yl)-N-ethyl-N-(3-methylbutyl)acetamide,
2-(3-Cyclohexyl-7-methoxy-2-oxoquinolin-1(2H)-yl)-N-(3,3-dimethylbutyl)-N-ethylacetamide,
2-(3-Cyclohexyl-7-methoxy-2-oxoquinolin-1(2H)-yl)-N-(3,3-dimethylbutyl)-N-propylacetamide,
2-(3-Cyclohexyl-7-methoxy-2-oxoquinolin-1(2H)-yl)-N-(2,2-dimethylpropyl)-N-ethylacetamide,
2-(3-Cyclohexyl-7-methoxy-2-oxoquinolin-1(2H)-yl)-N-(3-methylbutyl)-N-propylacetamide,
N-Butyl-2-(3-cyclohexyl-7-methoxy-2-oxoquinolin-1(2H)-yl)-N-(3-methylbutyl)acetamide,
2-(3-Cyclohexyl-7-methoxy-2-oxoquinolin-1(2H)-yl)-N-methyl-N-(3-methylbutyl)acetamide,
2-(3-Cyclohexyl-7-methoxy-2-oxoquinolin-1(2H)-yl)-N-(2-hydroxyethyl)-N-(3-methylbutyl)acetamide,
2-(3-Cyclohexyl-7-methoxy-2-oxoquinolin-1(2H)-yl)-N-ethyl-N-isobutylacetamide,
2-(3-Cyclohexyl-7-methoxy-2-oxoquinolin-1(2H)-yl)-N-isobutyl-N-propylacetamide,
N-Butyl-2-(3-cyclohexyl-7-methoxy-2-oxoquinolin-1(2H)-yl)-N-isobutylacetamide,
2-(3-Cyclohexyl-7-methoxy-2-oxoquinolin-1(2H)-yl)-N-(3-hydroxy-2,2-dimethylpropyl)-N-(3-methyl butyl)acetamide,
N-(3,3-Dimethylbutyl)-2-(7-methoxy-2-oxo-3-phenylquinolin-1(2H)-yl)-N-propylacetamide
N,N-Dibutyl-2-(7-methoxy-2-oxo-3-phenylquinolin-1(2H)-yl)acetamide,
N-(Cyclopropylmethyl)-2-(7-methoxy-2-oxo-3-phenylquinolin-1(2H)-yl)-N-propylacetamide,
N,N-Diisobutyl-2-(7-methoxy-2-oxo-3-phenylquinolin-1(2H)-yl)acetamide,
2-(7-Methoxy-2-oxo-3-phenylquinolin-1(2H)-yl)-N-bis(3-methylbutyl)acetamide,
N-Ethyl-2-(7-methoxy-2-oxo-3-phenylquinolin-1(2H)-yl)-N-(3-methylbutyl)acetamide,
N-(3,3-Dimethylbutyl)-N-ethyl-2-(7-methoxy-2-oxo-3-phenylquinolin-1(2H)-yl)acetamide,
N-(2,2-Dimethylpropyl)-N-ethyl-2-(7-methoxy-2-oxo-3-phenylquinolin-1(2H)-yl)acetamide,
2-(3-Ethyl-7-methoxy-2-oxoquinolin-1(2H)-yl)-N,N-bis(3-methylbutyl)acetamide,
N-(3,3-Dimethylbutyl)-2-(3-ethyl-7-methoxy-2-oxoquinolin-1(2H)-yl)-N-propylacetamide,
N,N-Bis(3,3-dimethylbutyl)-2-(3-ethyl-7-methoxy-2-oxoquinolin-1(2H)-yl)acetamide,
N,N-Bis(2,2-dimethylpropyl)-2-(3-ethyl-7-methoxy-2-oxoquinolin-1(2H)-yl)acetamide,
N-Butyl-N-(2,2-dimethylpropyl)-2-(3-ethyl-7-methoxy-2-oxoquinolin-1(2H)-yl)acetamide,
N-(2,2-Dimethylpropyl)-2-(3-ethyl-7-methoxy-2-oxoquinolin-1(2H)-yl)-N-pentylacetamide,
N-(2,2-Dimethylpropyl)-2-(3-ethyl-7-methoxy-2-oxoquinolin-1(2H)-yl)-N-(3-methylbutyl)acetamide,
N-Cyclopentyl-N-(2,2-dimethylpropyl)-2-(3-ethyl-7-methoxy-2-oxoquinolin-1(2H)-yl)acetamide,
N-(3,3-Dimethylbutyl)-N-(2,2-dimethylpropyl)-2-(3-ethyl-7-methoxy-2-oxoquinolin-1(2H)-yl)acetamide,
N-Butyl-N-(3,3-dimethylbutyl)-2-(3-ethyl-7-methoxy-2-oxoquinolin-1(2H)-yl)acetamide,
N-(3,3-Dimethylbutyl)-2-(3-ethyl-7-methoxy-2-oxoquinolin-1(2H)-yl)-N-pentylacetamide,
N-Cyclopentyl-N-(3,3-dimethylbutyl)-2-(3-ethyl-7-methoxy-2-oxoquinolin-1(2H)-yl)acetamide,
N-Butyl-2-(3-ethyl-7-methoxy-2-oxoquinolin-1(2H)-yl)-N-(3-methylbutyl)acetamide,
N,N-Dibutyl-2-(3-cyclopentyl-7-methoxy-2-oxoquinolin-1(2H)-yl)acetamide,
2-(3-Cyclopentyl-7-methoxy-2-oxoquinolin-1(2H)-yl)-N,N-dipropylacetamide,
2-(3-Cyclopentyl-7-methoxy-2-oxoquinolin-1(2H)-yl)-N-(cyclopropylmethyl)-N-propylacetamide,
N-Cyclohexyl-2-(3-cyclopentyl-7-methoxy-2-oxoquinolin-1(2H)-yl)-1′-ethylacetamide,
N-Butyl-2-(3-cyclopentyl-7-methoxy-2-oxoquinolin-1(2H)-yl)-N-ethylacetamide,
2-(3-Cyclopentyl-7-methoxy-2-oxoquinolin-1(2H)-yl)-N,N-diisobutylacetamide,
2-(3-Cyclopentyl-7-methoxy-2-oxoquinolin-1(2H)-yl)-N,N-bis(3-methylbutyl)acetamide,
2-(3-Cyclopentyl-7-methoxy-2-oxoquinolin-1(2H)-yl)-N-ethyl-N-(3-methylbutyl)acetamide,
N-Butyl-2-(3-cyclopentyl-7-methoxy-2-oxoquinolin-1(2H)-yl)-N-propylacetamide,
2-(3-Cyclopentyl-7-methoxy-2-oxoquinolin-1(2H)-yl)-N-(3,3-dimethylbutyl)-N-ethylacetamide,
2-(3-Cyclopentyl-7-methoxy-2-oxoquinolin-1(2H)-yl)-N-(3,3-dimethylbutyl)-N-propylacetamide,
2-(3-Cyclopentyl-7-methoxy-2-oxoquinolin-1(2H)-yl)-N-(2,2-dimethylpropyl)-N-ethylacetamide,
N-Cyclohexyl-2-(3-cyclopentyl-7-methoxy-2-oxoquinolin-1(2H)-yl)-N-isopropylacetamide,
2-(3-Cyclopentyl-7-methoxy-2-oxoquinolin-1(2H)-yl)-N,N-bis(3,3-dimethylbutyl)acetamide,
2-(3-Cyclopentyl-7-methoxy-2-oxoquinolin-1(2H)-yl)-N-octyl-N-pentylacetamide,
1-(sec-Butyl)-2-(3-cyclopentyl-7-methoxy-2-oxoquinolin-1(2H)-yl)-N-propylacetamide,
2-(3-Cyclopentyl-7-methoxy-2-oxoquinolin-1(2H)-yl)-N-ethyl-N-1,3-thiazol-2-ylacetamide,
(±)-3-Cyclopentyl-7-methoxy-1-{2-trans-octahydroisoquinolin-2(1H)-yl]-2-oxoethyl} quinolin-2(1H)-one,
N-Butyl-2-(3-cyclopentyl-7-methoxy-2-oxoquinolin-1(211)-yl)-N-(2,2-dimethylpropyl)acetamide,
2-(3-Cyclopentyl-7-methoxy-2-oxoquinolin-1(21H)-yl)-N-(2,2-dimethylpropyl)-N-pentylacetamide,
2-(3-Cyclopentyl-7-methoxy-2-oxoquinolin-1(2H)-yl)-N-(2,2-dimethylpropyl)-N-(3-methylbutyl)acetamide,
N-Cyclopentyl-2-(3-cyclopentyl-7-methoxy-2-oxoquinolin-1(2H)-yl)-N-(2,2-dimethylpropyl)acetamide,
2-(3-Cyclopentyl-7-methoxy-2-oxoquinolin-1(2H)-yl)-N-(3,3-dimethylbutyl)-N-(2,2-dimethylpropyl)acetamide,
N-Butyl-2-(3-cyclopentyl-7-methoxy-2-oxoquinolin-1(2H)-yl)-N-(3,3-dimethylbutyl)acetamide,
2-(3-Cyclopentyl-7-methoxy-2-oxoquinolin-1(2H)-yl)-N-(3,3-dimethylbutyl)-N-pentylacetamide,
N-Cyclopentyl-2-(3-cyclopentyl-7-methoxy-2-oxoquinolin-1(2H)-yl)-N-(3,3-dimethylbutyl)acetamide,
2-(3-Cyclopentyl-7-methoxy-2-oxoquinolin-1(2H)-yl)-N-(3,3-dimethylbutyl)-N-(3-methylbutyl)acetamide,
2-(3-Cyclopentyl-7-methoxy-2-oxoquinolin-1(2H)-yl)-N-(3-methylbutyl)-N-propylacetamide,
N-Butyl-2-(3-cyclopentyl-7-methoxy-2-oxoquinolin-1(2H)-yl)-N-(3-methylbutyl)acetamide,
2-(3-Cyclopentyl-7-methoxy-2-oxoquinolin-1(2H)-yl)-N-methyl-N-(3-methylbutyl)acetamide,
2-(3-Cyclopentyl-7-methoxy-2-oxoquinolin-1(2H)-yl)-N-(2-hydroxyethyl)-N-(3-methylbutyl)acetamide,
2-(3-Cyclopentyl-7-methoxy-2-oxoquinolin-1(2H)-yl)-N-ethyl-N-isobutylacetamide,
2-(3-Cyclopentyl-7-methoxy-2-oxoquinolin-1(2H)-yl)-N-isobutyl-N-propylacetamide,
N-Butyl-2-(3-cyclopentyl-7-methoxy-2-oxoquinolin-1(2H)-yl)-N-isobutylacetamide,
2-(3-Cyclopentyl-7-methoxy-2-oxoquinolin-1(2H)-yl)-N-(3-hydroxy-2,2-dimethylpropyl)-N-(3-methylbutyl)acetamide,
2-(3-Cyclopentyl-7-methoxy-2-oxoquinolin-1(2H)-yl)-N-(5-hydroxy-4,4-dimethylpentyl)-N-(3-methylbutyl)acetamide,
2-(3-Cyclopentyl-7-methoxy-2-oxoquinolin-1(2H)-yl)-N-(3,3-dimethylbutyl)-N-(5-hydroxypentyl)acetamide,
N,N-Dibutyl-2-(2-tert-butyl-6-methoxy-3-oxopyrido[2,3-b]pyrazin-4(3H)-yl)acetamide,
2-(2-tert-Butyl-6-methoxy-3-oxopyrido[2,3-b]pyrazin-4(3R)-yl)-N-(3,3-dimethylbutyl)-N-propylacetamide,
2-(2-tert-Butyl-6-methoxy-3-oxopyrido[2,3-b]pyrazin-4(3H)-yl)-N-ethyl-N-(3-methylbutyl)acetamide,
2-(2-tert-Butyl-6-methoxy-oxopyrido[2,3-b]pyrazin-4(3H)-yl)-N,N-bis(3-methylbutyl)acetamide,
2-(2-tert-Butyl-6-methoxy-3-oxopyrido[2,3-b]pyrazin-4(3H)-yl)-N,N-diisobutylacetamide,
N-Butyl-2-(2-tert-butyl-6-methoxy-3-oxopyrido[2,3-b]pyrazin-4(3H)yl)-N-propylacetamide,
2-(2-tert-Butyl-6-methoxy-3-oxopyrido[2,3-b]pyrazin-4(3H)-yl)-N-(3,3-dimethylbutyl)-N-ethylacetamide
2-(2-tert-Butyl-6-methoxy-3-oxopyrido[2,3-b]pyrazin-4(3H)-yl)-N-ethyl-N-1,3-thiazol-2-ylacetamide,
2-(2-tert-Butyl-6-methoxy-3-oxopyrido[2,3-b]pyrazin-4(3H)-yl)-N,N-bis(2,2-dimethylpropyl)acetamide,
2-(3-tert-Butyl-7-methoxy-2-oxoquinoxalin-1(2H)-yl)-N-(3,3-dimethylbutyl)-N-propylacetamide,
N,N-Dibutyl-2-(3-tert-butyl-7-methoxy-2-oxoquinoxalin-1(2H)-yl)acetamide,
2-(3-tert-Butyl-7-methoxy-2-oxoquinoxalin-1(2H)-yl)-N,N-diisobutylacetamide,
N-Butyl-2-(3-tert-butyl-7-methoxy-2-oxoquinoxalin-1(2H)-yl)-N-propylacetamide,
2-(3-tert-Butyl-7-methoxy-2-oxoquinoxalin-1(2H)-yl)-N-(cyclopropylmethyl)-N-propylacetamide,
2-(3-tert-Butyl-7-methoxy-2-oxoquinoxalin-1(2H)-yl)-N,N-dipropylacetamide,
2-(3-tert-Butyl-7-methoxy-2-oxoquinoxalin-1(2H)-yl)-N-(3,3-dimethylbutyl)-N-ethylacetamide,
2-(3-tert-Butyl-7-methoxy-2-oxoquinoxalin-1(2H)-yl)-N-ethyl-N-(3-methylbutyl)acetamide,
2-(3-tert-Butyl-7-methoxy-2-oxoquinoxalin-1(2H)-yl)-N-(2,2-dimethylpropyl)-N-ethylacetamide,
N-Butyl-2-(3-tert-butyl-7-methoxy-2-oxoquinoxalin-1(2H)-yl)-N-ethylacetamide,
2-(3-tert-Butyl-7-methoxy-2-oxoquinoxalin-1(2H)-yl)-N-cyclohexyl-N-ethylacetamide,
2-(3-tert-Butyl-7-methoxy-2-oxoquinoxalin-1(2H)-yl)—N-ethyl-N-1,3-thiazol-2-ylacetamide,
3-tert-Butyl-7-methoxy-1-{2-cis-octahydroisoquinolin-2(1H)-yl]-2-oxoethyl} quinoxalin-2(1H)-one,
3-tert-Butyl-7-methoxy-1-{2-cis-octahydroisoquinolin-2(1H)-yl]-2-oxoethyl} quinoxalin-2(1H)-one,
2-(3-tert-Butyl-7-methoxy-2-oxoquinoxalin-1(2H)-yl)-N,N-bis(2,2-dimethylpropyl)acetamide,
2-(3-tert-Butyl-7-methoxy-2-oxoquinoxalin-1(2H)-yl)-N-(2-hydroxyethyl)-N-(3-methylbutyl)acetamide,
2-(3-tert-Butyl-7-methoxy-2-oxoquinoxalin-1(2H)-yl)-N-(3,3-dimethylbutyl)-N-(3-hydroxy-2,2-dimethylpropyl)acetamide,
2-(3-tert-Butyl-7-methoxy-2-oxoquinoxalin-1(2H)-yl)-N-(3-hydroxy-2,2-dimethylpropyl)-N-(3-methylbutyl)acetamide,
2-(3-tert-Butyl-7-methoxy-2-oxoquinoxalin-1(2H)-yl)-N-(3,3-dimethylbutyl)-N-(4-hydroxy-3,3-dimethylbutyl)acetamide,
2-(3-tert-Butyl-7-methoxy-2-oxoquinoxalin-1(2H)-yl)-N-(3,3-dimethylbutyl)-N-(2-hydroxyethyl)acetamide,
2-(3-tert-Butyl-7-methoxy-2-oxoquinoxalin-1(2H)-yl)-N-(3,3-dimethylbutyl)-N-(3-hydroxy-3-methylbutyl)acetamide,
2-(3-tert-Butyl-7-methoxy-2-oxoquinoxalin-1(2H)-yl)-N-(3,3-dimethylbutyl)-N-(3-methylbut-2-en-1-yl)acetamide,
2-(3-tert-Butyl-7-methoxy-2-oxoquinoxalin-1(2H)-yl)-N-(3,3-dimethylbutyl)-N-(2-hydroxy-2-methylpropyl)acetamide,
2-(3-tert-Butyl-7-methoxy-2-oxoquinoxalin-1(2H)-yl)-N-(3,3-dimethylbutyl)-N-(5-hydroxypentyl)acetamide,
3-[[(3-tert-Butyl-7-methoxy-2-oxoquinoxalin-1(2H)-yl)acetyl](3,3-dimethylbutyl)amino]-2,2-dimethylpropyl dihydrogen phosphate,
2-[[(3-tert-Butyl-7-methoxy-2-oxoquinoxalin-1(2H)-yl)acetyl](3,3-dimethylbutyl)amino]-1,1-dimethylethyl dihydrogen phosphate,
3-tert-Butyl-1-(3,3-dimethyl-2-oxobutyl)-7-methoxyquinoxalin-2(1H)-one,
3-tert-Butyl-1-(3,3-dimethyl-2-oxopentyl)-7-methoxyquinoxalin-2(1H)-one,
3-tert-Butyl-1-(3-ethyl-3-methyl-2-oxopentyl)-7-methoxyquinoxalin-2(1H)-one,
3-tert-Butyl-7-methoxy-1-(3-methyl-2-oxobutyl)quinoxalin-2(1H)-one,
3-tert-Butyl-1-(2-cyclohexyl-2-oxoethyl)-7-methoxyquinoxalin-2(1H)-one,
3-tert-Butyl-1-(2-cyclopentyl-2-oxoethyl)-7-methoxyquinoxalin-2(1H)-one,
1-[2-(1-Adamantyl)-2-oxoethyl]-3-tert-butyl-7-methoxyquinoxalin-2(1H)-one,
2-(3-Isopropyl-7-methoxy-2-oxoquinoxalin-1(2H)-yl)-N,N-bis(3-methylbutyl)acetamide,
2-[3-[4-(3-Hydroxypropyl)phenyl]-7-methoxy-2-oxoquinoxalin-1(2H)-yl]-N,N-bis(3-methylbutyl)acetamide,
N-(3,3-Dimethylbutyl)-2-[3-[4-(3-hydroxypropyl)phenyl]-7-methoxy-2-oxoquinoxalin-1(2H)-yl]-N-propylacetamide,
3,3-Dimethyl-2-oxobutyl 4-[4-(3,3-dimethyl-2-oxobutyl)-6-methoxy-3-oxo-3,4-dihydroquinoxalin-2-yl]-4-methylpentanoate,
2-[3-(4-Hydroxy-1,1-dimethylbutyl)-7-methoxy-2-oxoquinoxalin-1(2H)-yl]-N,N-bis(3-methylbutyl)acetamide,
2-(3-tert-Butyl-7-methoxy-2-oxo-1,8-naphthyridin-1(2H)-yl)-N-(3,3-dimethylbutyl)-N-propylacetamide,
or a pharmaceutically acceptable salt, enantiomer, diatereomer or mixture thereof.

7. A method for treating ocular hypertension or glaucoma comprising administration to a patient in need of such treatment a therapeutically effective amount of a compound of structural formula I of claim 1.

8. A method for treating macular edema, macular degeneration, increasing retinal and optic nerve head blood velocity, increasing retinal and optic nerve oxygen tension, and/or a neuroprotective effect comprising administration to a patient in need of such treatment a pharmaceutically effective amount of a compound of claim 1; or a pharmaceutically acceptable salt, ester including phosphate, enantiomer, diastereomer or mixture thereof.

9. A composition comprising a compound of formula I of claim 1 and a pharmaceutically acceptable carrier.

10. A composition according to claim 9 wherein one or more of an active ingredient belonging to the group consisting of: P-adrenergic blocking agent, parasympatho-mimetic agent, sympathomimetic agent, carbonic anhydrase inhibitor, EP4 agonist, a prostaglandin or derivative thereof, hypotensive lipid, neuroprotectant, and/or 5-HT2 receptor agonist is optionally added.
